(19) United States
(12) Patent Application Publication
(10) Pub. No.: US 2011/0014241 A1
(43) Pub. Date: Jan. 20, 2011

(54) Therapeutic Cancer Antigens

(75) Inventor: Edward P. Cohen, Chicago, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Board of Trustees of the Unversity Illinois, Urbana, IL (US)

(21) Appl. No.: 12/922,581

(22) PCT Filed: Mar. 13, 2009
(86) PCT No.: PCT/US09/37176

§ 371 Date: Sep. 14, 2010

Related U.S. Application Data
(60) Provisional application No. 61/036,713, filed on Mar. 14, 2008.

(51) Int. Cl. A61K 39/00; C12N 5/071; A61K 35/12; A61P 35/00; A61P 37/04

(52) U.S. Cl. 424/277.1; 435/325; 435/366; 424/93.7; 424/93.21

(57) ABSTRACT

The present invention relates to cells, pharmaceutical compositions, tumor vaccines, kits and methods for inhibiting cell proliferation or tumor growth in a mammal, specifically, the invention relates to cells that express a tumor-associated tumor antigen.

This application is related to, and claims the benefit of priority to, U.S. Provisional Patent Application Ser. No. 61/036,713, filed Mar. 14, 2008, the disclosure of which is herein incorporated by reference in its entirety.

This invention was made with U.S. Government support under National Institute of Dental and Craniofacial Research (NIDCR) grant number 1 R01 DEO13970-O1A2; thus, the United States Government has certain rights in this invention.

### BACKGROUND OF THE INVENTION

1. Field of the Invention

This application relates to the field of tumor vaccine and tumor therapy. Particularly, the application relates to cells, tumor vaccines, compositions, methods, kits, and processes useful for the preparation and use thereof and the cells, compositions and tumor vaccines described herein.

2. Description of Related Art

Each year, over one million people in the United States alone are diagnosed with cancer. Each year, approximately 500,000 women worldwide develop breast cancer, which is the second leading cause of cancer-related death in women. The frequency of death of patients with metastatic breast cancer has remained essentially constant for the last thirty years, in spite of more sensitive methods of detection, novel chemotherapeutic agents, and improved hormonal, surgical and radiotherapeutic techniques. The five-year survival is poor. Therefore, new treatment options for cancer in general, and breast cancer specifically, are urgently needed.

The concept of immunotherapy as an adjunct to conventional forms of treatment of various types of cancer (including metastatic breast cancer) has been proposed. See U.S. patent application, Publication No. US2007-0009501 and Bankhead, 2007, J Natl Cancer Inst. 99:836. At least one basis for this therapy involves activated cytotoxic T lymphocytes (CTL), which recognize and kill cancer cells, being generated in patients receiving various forms of immune-based therapies. In most instances, the immunity is directed toward unique MHC class I-restricted tumor associated antigens expressed by the malignant cells.

Multiple tumor-associated antigens have been identified in various human malignancies, inter alia, by comparing microarrays of primary cancers or cancer cell-lines with cells from the homologous nonmalignant tissue. See, for example, Yoshitake et al., 2004, Clin Cancer Res 10:6437-48; Russo et al., 2003, Oncogene 22:6497-507; Quraishi et al., 2007, Appl Immunohistochem Mol Morphol 15:45-9; and Teschendorff et al., 2007, Genome Biol 8:R157 [Epub ahead of print]. On the other hand, relatively few tumor antigens associated with breast cancer have been identified, some of which were identified from T cell clones from cancer patients. See WO 96/34951; and Boon et al., 1994, Annu Rev Immunol 12:337-65. The genome of cancer cell is notoriously unstable. Genetic mutations resulting in the aberrant or over-expression of markers commonly expressed by nonmalignant cells may underlie their tumor-specific immunogenic properties. MUC1, HER2/neu, p53 and NY-BR-1 are known to be aberrantly expressed by malignant breast cancer cells. See, for example, Xu et al., 2005, Immunol Cell Biol 83:440-8; Mukherjee et al., 2004, Breast Dis 20:53-63; Allen et al., 2007, J. Immunol 179:472-82; Svane et al., 2007, Cancer Immunol Immunother 56:1485-99; and Seil et al., 2007, Int J Cancer 120:2635-42.

Growth Receptor-Bound protein 10 (Grb10) is a member of the family of adaptor proteins that interact with various receptor tyrosine kinases. See, for example, Kebache et al., 2007, J Biol Chem 282:21873-83; and Dufresne et al., 2005, Endocrinology 146:4399-409. Attachment of the adaptor protein to the receptor activates Ras and results in gene activation through the mitogen-activated protein kinase (MAPK) cascade. Grb10 was found to be over-expressed in adenovirus-infected or transformed cells. See Guan et al., 2003, Virology 25:114-24. Grb10 as a potential target in tumor immune therapy, however, has never been recognized or suggested.

The current tumor immune therapy using tumor vaccine based on known tumor-associated antigens (TAAs) suffers several drawbacks. One of the concerns is that the immunotherapeutic properties of the TAAs are unpredictable, in the sense that a TAA does not always elicit tumor-specific immunity, and further, immunity to the identified antigen does not always result in rejection of the tumor. Another concern is that many tumor vaccines were prepared by transfer into dendritic cells extracts of tumor cells and may not express sufficient amounts of the TAAs. See, for example, Mu et al., 2005, Br J Cancer 93:749-56; Lee et al., 2005, J. Immunother 28:496-504; Hus et al., 2005, Leukemia 19:1621-27; and Hayashi et al., 2005, J Oncol 26:1313-9. Further, the isolation of syngeneic dendritic cell, a process known as leukapheresis, and the expansion of such dendritic cells in vitro are known to be difficult, expensive and challenging.

Thus, there is a need in the art for a more effective tumor vaccine, and methods for preparing a more effective tumor vaccine.

### SUMMARY OF THE INVENTION

This invention thus provides cells, tumor vaccines, compositions, kits and methods for preparing and using the same, directed towards inhibiting growth of a target cell or a tumor cell. In particular, the invention provides cells, tumor vaccines, and pharmaceutical compositions that are highly enriched for one or more tumor-associated antigens that characterize a patient's cancer. More specifically, the invention provides cells, tumor vaccines, and compositions for inducing immunity to the tumor-associated antigen Grb10, and methods of inhibiting growth of tumor cells that express Grb10.

In a first aspect, the present invention provides isolated mammalian cells that express a tumor-associated antigen, wherein the administration of the isolated mammalian cell to a mammal induces an immune response to the tumor-associated antigen in the mammal In preferred embodiment, the isolated mammalian cell is allogeneic to the mammal In certain embodiments, the isolated mammalian cells are modified to express the tumor-associated antigen. In certain preferred embodiments, the tumor-associated antigen is Grb10. In certain other embodiments, the tumor-associated antigen is Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product.

In another aspect, the invention provides therapeutic tumor vaccines comprising isolated mammalian cells according to the first aspect of the invention and a pharmaceutically acceptable carrier, diluent or adjuvant. In certain preferred embodiments, the isolated mammalian cell expresses tumor-associated antigen Grb10. In yet another aspect, the invention provides pharmaceutical compositions in an amount effective to inhibit growth of a target cell in a mammal, said compositions comprising isolated mammalian cells according to the first aspect of the invention and a pharmaceutically acceptable carrier, diluent or adjuvant. In certain preferred embodiments, the isolated mammalian cell expresses tumor-associated antigen Grb10.

In a further aspect the invention provides methods of inhibiting proliferation of a target cell in a mammal comprising administering to the mammal an effective amount of a pharmaceutical composition comprising an isolated mammalian cell according to the first aspect of the invention, wherein proliferation of the target cell is inhibited thereby. In certain preferred embodiments, the isolated mammalian cell expresses tumor-associated antigen Grb10.

In another aspect the invention provides methods of inhibiting proliferation of a target cell in a mammal comprising administering to a mammal an effective amount of isolated mammalian cells according to the first aspect, wherein the proliferation of the target cell is inhibited thereby. In certain embodiments, the isolated mammalian cells are allogeneic to the mammal In certain preferred embodiments, the isolated mammalian cell expresses tumor-associated antigen Grb10.

In yet another aspect, the invention provides methods of inhibiting tumor growth in a mammal comprising administering to a mammal an effective amount of isolated mammalian cells of the first aspect, wherein the tumor growth is inhibited thereby. In certain embodiments, the isolated mammalian cells are allogeneic to the mammal In certain preferred embodiments, the isolated mammalian cell expresses tumor-associated antigen Grb10.

In a further aspect the invention provides methods of enhancing an immune response to a tumor-associated antigen in a mammal comprising administering to a mammal an effective amount of isolated mammalian cells of the first aspect. In preferred embodiments, the isolated mammalian cells are allogeneic to the mammal In certain preferred embodiments, the isolated mammalian cell expresses tumor-associated antigen Grb 10.

In yet a further aspect, the invention provides kits for inhibiting proliferation of a target cell in a mammal, said kits comprising a pharmaceutical composition or a tumor vaccine and instructions for use, wherein the pharmaceutical composition or tumor vaccine comprises an effective amount of isolated mammalian cells expressing a tumor-associated antigen according to the first aspect. In preferred embodiments, the isolated mammalian cells comprising the pharmaceutical composition or tumor vaccine are allogeneic to the mammal In certain preferred embodiments, the isolated mammalian cell expresses tumor associated-antigen Grb10.

Specific embodiments of the present invention will become evident from the following more detailed description of certain preferred embodiments and the claims.

### BRIEF DESCRIPTION OF FIGURES

FIG. 1 depicts RT-PCR results of LM-IL-2K^{b} cells and cells from various control groups for expression of the gene specifying Grb10.

FIG. 2A depicts the number of T cells responsive to SB5b cells, as measured by ELISPOT IFN-γ assays. *, P<0.0002; **, P<0.001; and ***, P<0.001.

FIG. 2B depicts the number of T cells responsive to immunization, as measured by ELISPOT IFN-γ assays, in the presence of mAbs for CD8⁺, CD4⁻ or natural killer T (NK-T) cells and complement. *, P<0.05.

FIG. 3 depicts a bar graph representing the percent specific cytolysis of SB5b cells by cytotoxic T lymphocytes (CTLs) isolated from the spleens of tumor-bearing mice immunized with LM-IL-2K^{b}/Grb10 cells or with control cells, as measured by ⁵¹Cr-release cytotoxicity assays. Inset: 200, 100, and 50 represent Effector:Target (E:T) cell ratio.

FIG. 4A depicts the percent survival of tumor-bearing C3H/He mice immunized with LM-IL-2K^{b}/Grb10 cells or with control cells.

FIG. 4B depicts the percent survival of mice first immunized with LM-IL-2K^{b}/Grb10 cells or with control cells, and then injected one week after immunization with SB5b breast cancer cells.

### DETAILED DESCRIPTION OF THE INVENTION

The invention provides cells, tumor vaccines and pharmaceutical compositions that are highly enriched for one or more tumor-associated antigens for use in inhibiting the growth of a target cell or tumor cell that expresses the one or more tumor antigens. In certain advantageous embodiments, the cells, pharmaceutical compositions, or tumor vaccines to be administered to a mammal possess little or no toxicity to the mammal

All molecular biology and DNA recombination techniques described herein are well known to one of ordinary skill in the art and further described in books such as Molecular Cloning: A Laboratory Manual (Sambrook, et al., 1989, Cold Spring Harbor Laboratory Press), which is incorporated herein by reference for any purposes.

As used herein, the singular forms “a”, “an” and “the” include plural referents unless the context clearly dictates otherwise.

As used herein the term “tumor-associated antigen” (TAA) refers to molecules that are associated with or detectably expressed by premalignant or malignant cells or cell populations. The tumor-associated antigen may also be expressed in certain normal cells or tissues during at least part of the normal cellular life cycle; however, the tumor-associated antigens as referred herein are expressed at higher levels in the tumor cells. In certain embodiments, the tumor-associated antigens are expressed at least from about 5 to about 100-folds higher in tumor cells relative to the levels in corresponding normal cells. It was recognized by the Applicants that the higher levels of expression in tumor cells and thus higher levels of antigen presentation of the tumor-associated antigens on tumors cells, but not necessarily the exclusivity of their expression in tumor cells, contributes to the tumor-specific immunity of these TAA-based vaccines.

In accordance with the present invention, in a first aspect, an isolated mammalian cell expressing a tumor-associated antigen is provided, wherein the administration of the isolated mammalian cell to a mammal induces an immune response to the tumor-associated antigen. In certain preferred embodiments, the isolated mammalian cell is allogeneic to the mammal In certain most preferred embodiments, the tumor-associated antigen is Grb10; in other embodiments, the tumor-associated antigen is, instead of Grb10, Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product.

According to this aspect, the immune response induced in the mammal includes without limitation cellular immune responses mediated by cytotoxic T cells and NK cells, as well as hormonal immune responses mediated primarily by helper T cells and B cells. Techniques for analyzing the types of immune responses induced by the isolated mammalian cells, compositions and tumor vaccines of the invention described herein are well known in the art and are further described in this application.

As used herein the term “isolated mammalian cell” refers to any mammalian cell suitable for expressing a tumor-associated antigen. Cells suitable for use include but are not limited to human embryonic fibroblast MRC-5 cells (American Type Culture Collection [ATCC] No. CC1-171), skin fibroblasts from allogeneic individuals, foreskin fibroblasts from new-born infants. Preferably, the cells are not tumor cells; however, tumor cells can be used. In certain embodiment, the cells are immortalized cells that are easily amenable to propagation in tissue culture, transfection, and other manipulations in vitro. In certain embodiments, the cells are primary cells. In certain preferred embodiments, the cells are fibroblasts. Preferably, the isolated mammalian cell is allogeneic to a mammal to which the isolated mammalian cell is administered; in certain other embodiments, the isolated mammalian cell is syngeneic to the mammal

In certain embodiments, the mammal is a human, and the isolated mammalian cell is a human cell. In certain advantageous embodiments, the human cell is human embryonic fibroblast MRC-5 cell (ATCC No. CC1-171). For application in a human subject, the isolated human cells must be free of adventitious and infections agents.

In certain preferred embodiments, the isolated mammalian cell expresses Grb10 endogenously. In alternative embodiments, the isolated mammalian cells are modified to express exogenous Grb10. Accordingly, the isolated mammalian cells of these embodiments further comprise a recombinant construct that allows transient, or preferably stable, expression of exogenous Grb10. A recombinant construct is well known to one of ordinary skill in the art, including without limitation, a plasmid, a cosmid, a retroviral vector, or a polynucleotide that is capable or incapable of replication in a mammalian cell. In certain embodiments the recombinant construct transduced into the mammalian cell spontaneously incorporates into the mammalian cell genome. In other embodiments, the mammalian cell stably expressing the exogenous tumor antigen is selected by way of antibiotic resistance conferred by the recombinant construct. All transient and stable transfection techniques are well known to one of ordinary skill in the art.

In certain other embodiments, the isolated mammalian cell further comprises one or more recombinant constructs encoding at least one of exogenous immune regulatory protein including without limitation interleukin 2 (IL2), Granulocyte-macrophage colony-stimulating factor (GMCSF) or IL17, or other immune stimulatory proteins that are known to promote uptake of cells, particularly allogeneic cells, by dendritic cells. The sequence information for IL2, GMCSF and IL17 is publicly available to one of skill in the art. Exemplary GenBank Accession Numbers for each gene are further provided herein: IL2: BC116873 (SEQ ID NOs: 72, 73), BC116845 (mouse), NM_{—}000586 (SEQ ID NOs: 74, 75), BC070338 (human); GMCSF: X02333 (SEQ ID NOs: 76, 77), X03019 (mouse), M11220 (SEQ ID NOs: 78, 79), M11734, M10663 (human); and IL17A: NM_{—}010552 (SEQ ID NOs: 80, 81), BC119309, BC119303 (mouse), NM_{—}002190 (SEQ ID NOs: 82, 83), BC067505 (human), IL17B: NM_{—}019508, BC002271 (SEQ ID NOs: 84, 85) (mouse), NM_{—}014443 (SEQ ID NOs: 86, 87), BC113946 (human).

In other embodiments of the first aspect of the invention, the isolated mammalian cell expresses at least one of tumor-associated antigen Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product.

In certain preferred embodiments, the isolated mammalian cell is allogeneic to the mammal to which the isolated mammalian cell is administered; however, syngeneic cells are also contemplated within the scope of the invention. As used herein the term “allogeneic” refers to genetically different, and the term “syngeneic” refers to genetic identical, traits or characteristics. In certain embodiments, the isolated mammalian cell is allogeneic to a mammal to which the isolated mammalian cell is administered in that the isolated mammalian cell carries at least one different MHC class or non-MHC determinant than the cells of the mammal Suitable allogeneic MHC and non-MHC determinants include without limitation MHC class I and class II molecules, T cell receptors and B cell receptors. In certain embodiments, the isolated mammalian cell expressing a tumor-associated antigen is syngeneic to the mammal to which the isolated mammalian cell is administered.

Allogeneity of the isolated mammalian cells with respect to the recipient mammal promotes uptake of the isolated mammalian cells by dendritic cells in the mammal to which the cells are administered. Allogeneity can be obtained by modifying a syngeneic cell to express at least one allogeneic determinant. Accordingly, in certain embodiments, the isolated mammalian cell is syngeneic to a mammal to which the isolated mammalian cell is administered, wherein the isolated syngeneic mammalian cell expresses Grb10, or other tumor-associated antigen described herein, and further comprises a recombinant construct that encodes an MHC or non-MHC determinant that is allogeneic to the mammal In certain embodiments, the mammal is a cancer-bearing patient, and the syngeneic cell is isolated from the patient.

The present invention overcomes the drawbacks suffered by the current tumor immune therapy by enriching the tumor-associated antigens in the immunogenic cells or vaccines so that the immunogenic cells or vaccines are capable of inducing an immune response to the antigen in a mammal The use of tumor-associated antigen as tumor vaccine in general has been described previously. See for example, U.S. Pat. Nos. 5,759,535, 6,187,307, and 7,402,306; U.S. Publication No. 2008-0305131; and WO 98/03357 and WO 06/105,255. Based on previously described methods of screening tumor therapeutic antigens with further modifications as described herein, Applicants unexpectedly identified at least 20 genes that are differentially expressed in transduced cells selected for their ability to induce strong immunity in mice to the breast cancer cell line SB5b. Among these 20 genes is Grb10, which was over-expressed nearly 100-fold in the transduced cells that conferred to mice strong immunity to SB5b cells. Although Grb10 has been known to be expressed and associated with tumor cells, its role as an immunogenic tumor antigen capable of inducing tumor rejection in animals has not been recognized. Further, among the Grb family members that share structural and functional similarity, Grb10 was the only member identified by the screening. The coding sequence and protein sequence of mouse Grb10 are designated as SEQ ID NO:1 and SEQ ID NO:2, respectively. The coding sequence and protein sequence of human Grb10 are designated as SEQ ID NO:3 and SEQ ID NO:4, respectively. It is understood by one skilled in the art that the experimental procedures applied to the analysis of Grb10 can be applied to all other tumor-associated antigens described herein.

The use of isolated mammalian cells of the invention provides another advantage in that the cells can be manipulated and expanded in vitro before being injected into an animal The use of easily-culturable mammalian cells eliminates the need of isolating syngeneic dendritic cells from immunized mammals by leukapheresis and avoids the challenging task of culturing the syngeneic dendritic cells in vitro, before administering the cells to an animal as a vaccine.

The isolated mammalian cells of the invention can be adapted for administration into a mammal in a formulation comprising a pharmaceutically acceptable carrier, adjuvant, or diluent. Such materials can be used for modifying, maintaining or preserving, for example, the pH, osmolarity, cell viability, viscosity, clarity, color, isotonicity, odor, sterility, stability, rate of dissolution or release, adsorption or penetration of the formulation. Suitable materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Acceptable carriers, adjuvants, or diluents include any materials suitable for use in the formulation.

In another aspect, the present invention provides tumor vaccines comprising an isolated mammalian cell of the first aspect of the invention and a pharmaceutically acceptable carrier, diluent or adjuvant. In certain preferred embodiments, the tumor vaccine is a therapeutic tumor vaccine. In certain other embodiments, the tumor vaccine is a preventive tumor vaccine. In certain preferred embodiments, the isolated mammalian cell expresses tumor-associated antigen Grb10. In certain other embodiments, the isolated mammalian cell expresses a tumor-associated antigen that is, instead of Grb10, Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product. In yet other embodiments, the isolated mammalian cell further comprises a recombinant construct encoding exogenous IL2, GMCSF, or IL17.

As used herein the term “therapeutic tumor vaccine” refers to a vaccine wherein administration thereof to an animal having a tumor results in reduction in tumor growth. Tumor growth can be measured by, including without limitation, total tumor cell number and tumor volume. Tumor volume can be determined by various known procedures, e.g., by obtaining two dimensional measurements with a dial caliper. Alternatively, the survival of tumor-bearing animals can be used as an indication of tumor growth.

In certain advantageous embodiments, the therapeutic tumor vaccine further comprises one or more immune stimulatory agents, which include, without limitation, IL2, GMCSF, and IL17.

The tumors contemplated by the present invention, against which the immune response is induced, or which can be prevented or treated, include any tumor expressing a tumor-associated antigen described herein and are not limited to melanoma, lymphoma, plasmocytoma, sarcoma, glioma, thymoma, leukemias, breast cancer, prostate cancer, colon cancer, esophageal cancer, brain cancer, lung cancer, ovary cancer, cervical cancer, hepatoma, and other neoplasms known in the art. In preferred embodiments the tumor is a breast cancer tumor. In certain preferred embodiments, the isolated mammalian cell of the tumor vaccine is allogeneic to the tumor-bearing patient.

In yet another aspect the invention provides pharmaceutical compositions comprising an isolated mammalian cell of the invention, in an amount effective to inhibit proliferation of a target cell in a mammal, and a pharmaceutically acceptable carrier, adjuvant, or diluent. In certain preferred embodiments, the isolated mammalian cell is allogeneic to the mammal In certain preferred embodiments, the isolated mammalian cell expresses tumor-associated antigen Grb10. In certain other embodiments, the isolated mammalian cell expresses tumor-associated antigen Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product. In further embodiments, the isolated mammalian cell further comprises a recombinant construct encoding exogenous IL2, GMCSF, or IL17.

As used herein, the term “inhibiting proliferation of a target cell” refers to a reduction of the size or number of the target cell, or an inhibition of growth in the size, or number of the target cell. In certain embodiments, the target cell expresses Grb10; in certain other embodiments, the target cell is a tumor cell expressing Grb10.

In certain other embodiments of this aspect, the pharmaceutical composition further comprises an immune stimulatory agent as described herein.

The isolated mammalian cells, pharmaceutical compositions and tumor vaccines of the invention can be used as a supplemental tumor therapy, administered before or after conventional tumor therapy, including without limitation, chemotherapy and radiation therapy. In certain preferred embodiments, the pharmaceutical composition or tumor vaccine is administered before or after chemotherapy to eliminate residue tumor cells.

In yet another aspect the invention provides methods of inhibiting proliferation of a target cell or a tumor cell in a mammal comprising administering to the mammal an effective amount of the isolated mammalian cell of the invention, pharmaceutical composition or tumor vaccine containing the same, wherein the isolated mammalian cell expresses a tumor-associated antigen as described herein, and wherein proliferation of the target cell or a tumor cell is inhibited thereby. In certain advantageous embodiments, the isolated mammalian cells are allogeneic to the mammal In certain preferred embodiments, the tumor-associated antigen is Grb10; in other embodiments, the tumor-associated antigen is Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product. In other preferred embodiments, the mammal is a human.

In a further aspect the invention provides methods of inhibiting tumor growth in a mammal comprising administering to a mammal an effective amount of an isolated mammalian cell of the invention, or pharmaceutical composition or tumor vaccine containing the same, wherein the isolated mammalian cell expresses a tumor-associated antigen as described herein and wherein the tumor growth is inhibited thereby. In certain embodiments, the isolated mammalian cell is allogeneic or syngeneic to the mammal In certain preferred embodiments, the tumor-associated antigen is Grb10; in other embodiments, the tumor-associated antigen is Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product. In other preferred embodiments, the mammal is a human.

As used herein, the term “target cell” refers to a cell in a mammal, the proliferation of which is inhibited by the administration to the mammal the isolated mammalian cell of the invention, or pharmaceutical composition or tumor vaccine containing the same. In certain embodiments of this aspect, the target cell expresses tumor-associated antigens described herein, preferably Grb10; in certain preferred embodiments, the target cell is a tumor cell expressing tumor-associated antigens described herein, preferably Grb10. The tumor cells can be any tumor cell as described herein, including, but not limited to, melanoma, lymphoma, plasmacytoma, sarcoma, glioma, thymoma, leukemia, breast cancer, prostate cancer, colon cancer, esophageal cancer, brain cancer, lung cancer, ovarian cancer, cervical cancer or hepatoma cell. In certain preferred embodiments the tumor cell is a breast cancer cell. In further advantageous embodiments, the isolated mammalian cell is allogeneic to the tumor cell.

In certain embodiments, the inhibition of proliferation of the target cell or the tumor cell is mediated by an immune response to the tumor-associated antigen, preferably to Grb10, induced after the pharmaceutical composition, tumor vaccine or isolated mammalian cell of the invention is administered to the mammal The immunity includes without limitation CD4 cell-, CD8 cell- or NK cell-mediated immunity.

As used herein, when “an effective amount” is indicated, the precise amount of the pharmaceutical composition or isolated mammalian cell can be determined by a physician with consideration of individual differences in age, weight, tumor size, extent of infection or metastasis, and condition of the patient. It can generally be stated that a therapeutic composition comprising the isolated mammalian cells should be preferably administered in an amount of at least about 1×10³ to about 5×10⁹ cells, preferably about 1×10⁴ to about 5×10⁶ cells, most preferably about 1×10⁵ to about 5×10⁶ cells, per dose.

The administration of the pharmaceutical compositions, tumor vaccines, or isolated mammalian cells of the invention can be carried out in any convenient manner, including without limitation injection, transfusion, implantation or transplantation. Preferably, compositions, tumor vaccines or isolated mammalian cells of the invention are administered to a patient by subcutaneous (s.c.), intraperitoneal (i.p.), intra-arterial (i.a.), intradermal (i.d.) or intravenous (i.v.) injection.

In a further aspect the invention provides a method of enhancing an immune response to a tumor-associated antigen described herein. Preferably, the invention provides a method of enhancing an immune response to tumor-associated antigen Grb10 in a mammal comprising administering to a mammal an effect amount of an isolated mammalian cell, wherein the isolated mammalian cell expresses Grb10. In other embodiments of this aspect, the isolated mammalian cell expresses a tumor-associated antigen that is Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product. In certain embodiments, the isolated mammalian cell is allogeneic to the mammal

In yet a further aspect, the invention provides kits for inhibiting proliferation of a target cell in a mammal, said kits comprising a pharmaceutical composition or a tumor vaccine and instructions for use, wherein the pharmaceutical composition or tumor vaccine comprises an effective amount of isolated mammalian cells expressing a tumor-associated antigen according to the first aspect. In preferred embodiments, the isolated mammalian cells comprising the pharmaceutical composition or tumor vaccine are allogeneic to the mammal In certain preferred embodiments, the isolated mammalian cell expresses tumor associated-antigen Grb10; in other embodiments, the tumor-associated antigen is Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product.

The disclosures of all publications and patent documents cited throughout the present specification are herein incorporated by reference in their entirety.

The Examples, which follow, are illustrative of specific embodiments of the invention, and various uses thereof. They are set forth for explanatory purposes only, and are not to be taken as limiting the invention.

### Examples

### Example 1

### IL-2 Secretion and Expression of H-2K^{b}-Determinants by Modified LM Fibroblasts, used as Recipients of cDNA Libraries from Mammary Carcinoma Cells

LM fibroblasts, of C3H/He mouse origin, were obtained from the American Type Culture Collection (ATCC No. CCL-1.4). Cells were maintained at 37° C. in a humidified 7% CO₂/air atmosphere in DMEM (Gibco BRL, Grand Island, N.Y.) supplemented with 10% heat inactivated fetal bovine serum (FBS) and antibiotics (Gibco BRL) (growth medium). The fibroblasts were modified to secrete IL-2 before transfection (LM-IL-2 cells) as a means of augmenting their non-specific immunogenic properties, as described previously (Chopra et al., 2006, Int. J. Cancer 119: 339-348).

Allogeneic class I-determinants are strong immune adjuvants. To stimulate uptake of the vaccine by dendritic cells of the tumor-bearing host, and to ensure rejection, fibroblasts (H-2^{k}) were modified to express H-2K^{b}-class I-determinants, allogeneic in C3H/He mice (LM-IL-2K^{b} cells).

Among other advantages, the use of a nonmalignant cell line as the recipient of the cDNA library enabled the recipient cells to be conveniently modified beforehand to augment their nonspecific immunogenic properties. In this instance, the fibroblasts, of C3H/He mouse origin, were modified to secrete IL-2, a T cell growth factor, and to express H-2K^{b}-determinants, allogeneic in C3H/He mice (LM-IL-2K^{b} cells). The transduced cells formed 3.3+/−0.3 ng IL-2/10⁶ cells/48 hrs. Non-transduced fibroblasts failed to form detectable quantities of IL-2. The mean fluorescence index (MFI) of LM cells transduced with pBR327H-2K^{b}, the plasmid that specified H-2K^{b}, stained with fluorescein-conjugated H-2K^{b} mAbs was significantly higher than that of cells stained with the isotope control sera (1.0+/−0.2 and 0.3+/−0.1 respectively). Secretion of IL-2 and expression of H-2K^{b}-determinants by the modified cells were essentially unchanged after three months of continuous culture (data not shown).

### Example 2

### Microarray Analysis of Cellular Vaccines Enriched for Transduced Fibroblasts Demonstrated that Grb10 was Relatively Overrepresented in Cells that Induced Immunity to SB5b Cells

To prepare cDNA libraries for transduction into modified fibroblasts, guanidine isothiocyanate was used to recover total RNA from SB5b cells or from B16F1 melanoma cells. SB5b cells were a breast cancer cell line established from an adenocarcinoma that arose spontaneously in the mammary gland of a C3H/He mouse in our animal colony. B16F1 cells, a melanoma cell line of C57BL/6 mouse origin (H-2^{b}), were obtained from the ATCC (ATCC No. MGC-13906). Cells were maintained at 37° C. in a humidified 7% CO₂/air atmosphere in DMEM (Gibco BRL, Grand Island, N.Y.) supplemented with 10% heat inactivated fetal bovine serum (FBS) and antibiotics (Gibco BRL) (growth medium). Pathogen-free C3H/He female mice (H-2^{k}) between 10 to 14 weeks old were from the Jackson Laboratory (Bar Harbor, Me.) and were maintained according to NIH Guidelines for the Care and Use of Laboratory Animals.

mRNA, derived from approximately 1×10⁷ cells, was isolated using an mRNA isolation system (Promega, Madison, Wis.). cDNA-expression libraries were constructed with a Lambda Zap vector using a cDNA library kit (Stratgene, La Jolla, Calif.). cDNAs greater than 0.5 kb in length were selected by size fractionation via gel filtration and directionally cloned into a pBK-CMV vector with an EcoRI restriction site at the 5′ end and an XhoI site at the 3′ end. The expression libraries yielded approximately 4×10⁵ PFU/μg cDNA with an individual cDNA insert. The size distribution of the cDNA transduced into the modified fibroblasts was 0.5-7.0 kb.

To prepare cDNA-based cellular vaccines, LM-IL-2K^{b} cells were transduced with a cDNA library from SB5b cells, or, for use as a specificity control, with a cDNA library from B16F1 cells, using Lipofectamine 2000 (Invitrogen, Carlsbad, Calif.) to aid cDNA uptake. In brief, 30 μg of cDNA from either of the cell types were mixed with 3 μg pcDNA6/Bla (Invitrogen), a plasmid specifying a gene conferring resistance to blasticidin, an antibiotic used for selection (CalBiochem, San Diego, Calif.). Afterward, the cDNA/pcDNA6/Bla mixture was added to Lipofectamine 2000 and then to 2.0×10⁷ LM-IL-2K^{b} cells divided 24 hours previously into four 100 mm plastic cell culture dishes. After incubation for 18 hours, the cells were divided into sixteen 100 mm dishes, and incubated for 14 days in fresh growth medium containing 5 μg/mL blasticidin and 500 μg/mL G418. The surviving blasticin/G418-resistant cells (at least 2×10⁶ colonies) were pooled and maintained as cell lines for use in the experiments described herein (designated as LM-IL-2K^{b}/cSB5b and LM-IL-2K^{b}/cB16F1 cells respectively). For use as a control, the same procedure was followed except that the fibroblasts were transduced with pcDNA/Bla alone (LM-IL-2K^{b}/−cells).

Since only a small proportion of the transduced cell population was expected to have incorporated cDNAs that included genes specifying tumor-associated antigens (TAA), a unique strategy was used to enrich the vaccine for TAA-positive cells, as described previously (Chopra et al., 2006, Int. J. Cancer 119: 339-348). In brief, aliquots of the suspension of transduced cells were added to each of ten wells of a 96-well plate. Each pool contained a starting inoculum of 1×10³ cells. Wells containing higher numbers of TAA-positive cells were detected by comparing the response of C3H/He mice to immunization with cells from the individual pools, as determined by both enzyme-linked immunospot interferon gamma (ELISPOT IFN-γ) and ⁵¹Cr-release cytotoxicity assays. Mouse ELISPOT IFN-γ assays were used to determine the number of responding T cells in mice immunized with the transduced fibroblasts. The spots were counted by computer-assisted image analysis (ImmunoSpot Series 2 analyzer: Cellular Technology Limited, Cleveland, Ohio). For ⁵¹Cr-release cytotoxicity assays, mononuclear cells from the spleens of C3H/He mice immunized with the cDNA-transduced cells were isolated by Ficoll-Hypaque density gradient centrifugation. After washing, the cells were co-cultured at 37° C. with mitomycin C-treated (45 min, 50 μg/mL) SB5b cells for 5 days (ratio of spleen cells:SB5b cells=30:1). Afterward, the population that failed to adhere to the plastic cell culture flasks was collected and used as effector cells for cytotoxicity determinations. Spleen cell mediated cytotoxicity was determined in a standard ⁵¹Cr-release assay, using ^{|}Cr-labeled SB5b cells as targets in the reaction. The percent specific cytolysis was calculated as:

$$\% Cytolysis = \frac{( Experimental {}^{51} Cr release ) - ( Spontaneous {}^{51} Cr release )}{( Maximum {}^{51} Cr release ) - ( Spontaneous {}^{51} Cr release )} \times 100$$

The spontaneous release of ⁵¹Cr was less than 15% of the total release in each instance.

To obtain a sufficient number of cells for immunization, cells from the individual pools were allowed to increase to ˜5×10⁷ through periodic transfers to larger culture plates and eventually cell culture flasks. An aliquot of each of the expanded cell populations was maintained frozen/viable (for later recovery). The remaining portion was used for immunization. Frozen cells derived from the pool that stimulated immunity to the breast cancer cells to the greatest extent (immuno^{high}) and, for use as a control, from the pool that induced immunity to SB5b cells to the least extent (immuno^{low}), were recovered, reestablished in culture and subjected to additional rounds of positive or negative immune selection (Chopra et al., 2006, Int. J. Cancer 119: 339-348). As an additional control, one pool was subjected to neither positive nor negative selection (master pool).

After five rounds of selection, cRNA microarrays were used to compare the gene expression profiles of cells in the immuno^{high} and immuno^{low} pools, as described previously (O'Sullivan et al., 2007, Cancer Gene Ther. 14: 389-398). Twenty genes were relatively overrepresented in cells from the immuno^{high} pool (Table 1). The gene for Grb10 was approximately 100-fold over-represented. Other highly overrepresented genes included tripartite motif protein 13 (71.5-fold over-represented), serum amyloid A3 (44-fold over-represented) and Xir-related meiosis regulated gene (39-fold over-represented), demonstrating that multiple genes in the immuno^{high} pool of transduced cells specified an array of immunogenic TAA.

TABLE 1
Genes relatively over represented in the cellular vaccine selected for its
enhanced immunotherapeutic properties in mice with breast cancer

| H/L | Description |
|---|---|
| 99.92 | Growth factor receptor bound protein 10 |
| | Mouse (GenBank Accession No. BC016111) SEQ ID NOs: 1 and 2 |
| | Human (GenBank Accession No. BC024285) SEQ ID NOs: 3 and 4 |
| 71.57 | Tripartite motif protein 13 |
| | Mouse (GenBank Accession No. BC145915) SEQ ID NOs: 7 and 8 |
| | Human (GenBank Accession No. BC063407) SEQ ID NOs: 9 and 10 |
| 44.25 | Serum amyloid A 3 |
| | Mouse (GenBank Accession No. BC055885) SEQ ID NOs: 11 and 12 |
| | Human (GenBank Accession No. NM_000331) SEQ ID NOs: 13 and 14 |
| 39.91 | Xlr-related, meiosis regulated |
| | Mouse (GenBank Accession No. NM_001109970) SEQ ID NOs: 15 and 16 |
| 35.22 | Pentaxin related gene |
| | Mouse (GenBank Accession No. NM_008987) SEQ ID NOs: 17 and 18 |
| | Human (GenBank Accession No. BC039733) SEQ ID NOs: 19 and 20 |
| 29.81 | CD36 antigen |
| | Mouse (GenBank Accession No. NM_007643) SEQ ID NOs: 21 and 22 |
| | Human (GenBank Accession No. M24795) SEQ ID NOs: 23 and 24 |
| 24.65 | RIKEN cDNA 9030625A04 gene |
| | Mouse (GenBank Accession No. NM_172488) SEQ ID NOs: 25 and 26 |
| 22.54 | Prostaglandin-endoperoxide synthase 2 |
| | Mouse (GenBank Accession No. BC052900) SEQ ID NOs: 27 and 28 |
| | Human (GenBank Accession No. BC013734) SEQ ID NOs: 29 and 30 |
| 15.32 | RIKEN cDNA E030003E18 gene |
| | Mouse (GenBank Accession No. XM_988020) SEQ ID NOs: 31 and 32 |
| 13.47 | Tumor-associated calcium signal transducer 1 |
| | Mouse (GenBank Accession No. BC094465) SEQ ID NOs: 33 and 34 |
| | Human (GenBank Accession No. BC014785) SEQ ID NOs: 35 and 36 |
| 12.70 | RIKEN cDNA 2310005E10 gene |
| | Mouse (GenBank Accession No. BC037690) SEQ ID NOs: 37 and 38 |
| 12.17 | DEAD (Asp-Glu-Ala-Asp) box polypeptide 25 |
| | Mouse (GenBank Accession No. BC024852) SEQ ID NOs: 39 and 40 |
| | Human (GenBank Accession No. BC050360) SEQ ID NOs: 41 and 42 |
| 11.51 | Neuropeptide Y receptor Y1 |
| | Mouse (GenBank Accession No. NM_010934) SEQ ID NOs: 43 and 44 |
| | Human (GenBank Accession No. NM_000909) SEQ ID NOs: 45 and 46 |
| 9.93 | GRP1 (general receptor for phosphoinositides 1)-associated scaffold protein |
| | Mouse (GenBank Accession No. NM_019518) SEQ ID NOs: 47 and 48 |
| | Human (GenBank Accession No. NM_181711) SEQ ID NOs: 49 and 50 |
| 9.53 | Nuclear receptor subfamily 4, group A, member 1 |
| | Mouse (GenBank Accession No. BC004770) SEQ ID NOs: 51 and 52 |
| | Human (GenBank Accession No. BC016147) SEQ ID NOs: 53 and 54 |
| 9.39 | SRY-box containing gene 5 |
| | Mouse (GenBank Accession No. BC110478) SEQ ID NOs: 55 and 56 |
| | Human (GenBank Accession No. BC060773) SEQ ID NOs: 57 and 58 |
| 8.27 | Carbonic anhydrase 9 |
| | Mouse (GenBank Accession No. NM_139305) SEQ ID NOs: 59 and 60 |
| | Human (GenBank Accession No. BC014950) SEQ ID NOs: 61 and 62 |
| 7.24 | Aldehyde dehydrogenase family 1, subfamily A7 |
| | Mouse (GenBank Accession No. NM_011921) SEQ ID NOs: 63 and 64 |
| | Human (GenBank Accession No. NM_000689) SEQ ID NOs: 65 and 66 |
| 7.16 | Thymoma viral proto-oncogene 3 |
| | Mouse (GenBank Accession No. BC066861) SEQ ID NOs: 67 and 68 |
| | Human (GenBank Accession No. BC121154) SEQ ID NOs: 69 and 70 |
| 7.08 | RIKEN cDNA D130020G16 gene |
| | Mouse (GenBank Accession No. AK051236) SEQ ID NO: 71 |

NOTE:
Comparative microarrays of immuno^{high} (after five rounds of positive immune selection) and immuno^{low} (after five rounds of negative immune-selection) LM-IL-2K^{b}/SB5b cells.

### Example 3

### RT-PCR of Grb10, a Candidate Gene Specifying a Breast Cancer Antigen, Identified by Comparing Microarrays of Enriched and Non-Enriched Vaccines

cDNA or cRNA microarray (GE Healthcare/Amersham Biosciences CODELINK™ UniSet Mouse 20K I Bioarray, GE Healthcare Bioscience Corp. Piscataway, N.J.) analysis demonstrated that Grb10 was highly overrepresented in cells from immuno^{high} pools (see Example 2). RT-PCR was used to determine if the gene was expressed. Approximately 6×10⁶ cells from the immuno^{high} pool in monolayer culture were disrupted and homogenized. One volume of 70% ethanol was added before the extracts were loaded onto RNeasy mini columns. RT-PCR was performed on RNA eluted from the column with a one-step RT-PCR kit (Qiagen, Valencia, Calif.), according to the manufacturer's instructions. One μg RNA was mixed with buffer containing 1.25 mM MgCl₂, 40 μM dNTPs, 0.6 μM of each forward and backward primers and 2 μL of a mixture containing reverse transcriptase and Taq polymerase. The reverse transcriptase reaction was at 50° C. for 45 min. The PCR reaction was at 94° C. The denaturation step was for 2 min at 58° C. The annealing step was for 1 min. at 72° C. and extension was for 2 min. for 35 cycles. A DNA Thermal Cycler 480 (Perkin-Elmer, Wellesley, Mass.) was used for the reactions. The primers used were: Grb10 forward: 5′-CGTGGTCCAGTGGAGAGTA (SEQ ID NO: 5); backward: 5′-TCCGGTCTTCGGCGTAACTGA (SEQ ID NO: 6).

An expression vector that specified the gene for Grb10 was prepared by ligation of the gene into a pCR 2.1 vector using a TA 2.1 cloning kit (Invitrogen, Carlsbad, Calif.). The mouse coding sequence and protein sequence of Grb10 are designated as SEQ ID NO:1 and SEQ ID NO:2, respectively. In brief, 50 ng pCR2.1 and 2 μL of the PCR-product containing 10 ng Grb10 was mixed with buffer and 1 μL T4 DNA ligase in 10 μL total volume and incubated at 14° C. for 4 hrs. 5 μL of the ligation-mixture containing pCR2.1/Grb10 was transferred into 50 μL DH5α competent cells followed by 30 min incubation on ice. Afterward, the cells were subjected to a 20 second heat-shock at 37° C. and 2 min additional incubation on ice. As a control, pUC 19 DNA (5 μL) was also transferred into DH5α competent cells. The transformation complex was mixed with 950 μL Super Optimal broth with Catabolite repression (SOC) medium and incubated at 37° C. for 1 hr. The cell pellets were plated on LB agar plates containing 100 μg/mL ampicillin and 1 mg X-Gal and incubated at 37° C. overnight. White colonies indicating insertion of the Grb10 gene in the lacZ site of pCR2.1 were selected and amplified. DNA from each amplified clone was extracted and digested with Eco RI enzyme to verify the presence of the 430 bp portion of the Grb10 gene. The resulting 430 bp band was recovered from the gel and purified from a Gel-purification kit (Qiagen, Valencia, Calif.). The 430 bp portion of the gene was ligated into the expression vector pcDNA6/V5-HisA (Invitrogen, Carlsbad, Calif.). In brief, 170 ng of pcDNA6/V5-HisA digested with EcoRI was mixed with 30 ng Grb10 and 3 μL T4 DNA ligase with buffer and incubated at 14° C. for 4 hrs. Ligation mixtures containing pcDNA6N5-HisA/Grb10 were transformed into 50 μL of chemically competent E coli DH5a cells with 30 min incubation on ice followed by 20 sec heat-shock at 37° C. and 2 min additional incubation on ice. As a control, 5 μL of pUC 19 DNA was also transferred into DH5α competent cells. The transformation complex was mixed with 950 μL SOC medium and incubated at 37° C. for 1 hr. The cell pellets were plated on LB agar plates containing 100 μg/mL ampicillin and incubated at 37° C. overnight. Colonies were selected and amplified in 2 mL cultures for DNA isolation and Grb10 verification through EcoRI digestion. The identified pcDNA6N5-HisA/Grb10 clone was amplified in 2 liter cultures and 1.26 mg of pcDNA6N5-HisA/Grb10 DNA was obtained, using a plasmid maxi prep kit (Qiagen, Valencia, Calif.).

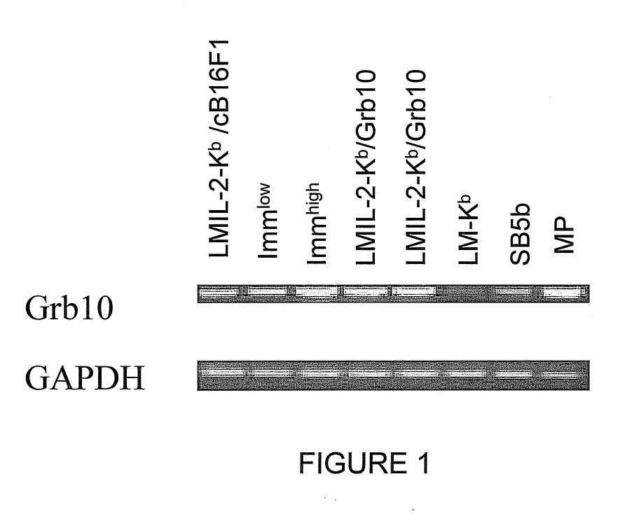

A vaccine for breast cancer was prepared by transduction of LM-IL-2K^{b} cells with pcDNA6N5-HisA/Grb10, an expression vector that specified Grb10. As a first step, RT-PCR was used to determine if the transduced cells expressed the gene specifying Grb10. These results are shown in FIG. 1: lane 1, LM-IL-2K^{b}/cB16F1 (LM-IL-2K^{b} cells transfected with pcDNA6/cB16F1); lane 2, immune^{low} (cells from the immune^{low} pool after five rounds of negative immune-selection); lane 3, immuno^{high} (cells from the immuno^{high} pool after five rounds of positive immune-selection); lane 4, LM-IL-2K^{b}/Grb10 (LM-IL-2K^{b} cells transfected with pcDNA6/Grb10); lane 5, LM-IL-2K^{b}/Grb10 (LM-IL-2K^{b} cells transfected with pcDNA6/Grb10); lane 6, LM-K^{b} (LM cells transduced with a plasmid specifying K^{b}-determinants); lane 7, SB5b (SB5b breast cancer cells); lane 8, MP (cells from the non-enriched master pool of LM-IL-2K^{b}/cSB5b cells). GADPH, glyceraldehyde-3 -phosphate dehydrogenase.

As indicated, Grb10 was strongly expressed by LM-IL-2K^{b}/Grb10 cells (lanes 4-5, duplicates) by cells from the immuno^{high} pool (lane 3) and by cells from the non-enriched master pool (lane 8). Grb10 was also expressed, but to a lesser extent, by cells from the immune^{low} pool (lane 2), by the breast cancer cells, by non transduced fibroblasts (LM-IL-2-K^{b}) (lane 6) and by fibroblasts transduced with a cDNA library from B16F1 melanoma cells (LM-IL-2K^{b}/cB16F1) (lane 1). Quantitative RT-PCR was used to compare the relative expression levels of Grb10 by cells from the immuno^{high} and the immuno^{low} pools. The results indicated that the expression of Grb10 by cells from the immuno^{high} pool was 75.6 fold higher than that of cells from the immuno^{low} pool (data not shown).

### Example 4

### Immunity to Breast Cancer in Mice Immunized with a Vaccine Prepared by Transfection of Modified Fibroblasts with the Vector Specifying Grb10 (LM-IL-2K^{b}/Grb10 Cells)

This vaccine was prepared by transfection of modified fibroblasts with the Grb10-vector, according to an analogous procedure reported previously (O'Sullivan et al., 2007, Cancer Gene Ther. 14: 389-398). In brief, 2×10⁶ LM-IL-2K^{b} cells were added to four 100 mm plates in minimal growth medium (MGM) (Invitrogen, Carlsbad, Calif.) without antibiotics. Afterward, 30 μg of pcDNA6N5-HisA/Grb10 DNA in 2 mL Opti medium (Invitrogen, Carlsbad, Calif.) was mixed with 100 μL Lipofectamine 2000 (Invitrogen, Carlsbad, Calif.), followed by incubation for 20° C. at RT. One mL of the 4 mL transfection complex (pcDNA6N5-HisA/Grb10 DNA and Lipofectamine 2000) was added to each of the four plates and incubated overnight at 37° C. in a 7% CO₂/air incubator. The number of plates was expanded to sixteen. The transduced cells were selected in growth medium containing 5 μg/mL blasticidin. The blasticidin-resistant cells were allowed to proliferate for 7 additional days and pooled. One half of the cell suspension was maintained frozen/viable; the remaining portion was maintained at 37° C. in a 7% CO₂/air incubator in selection medium. For use as a control, the same procedure was followed except that the LM-IL-2K^{b} cells were transduced with the “empty” vector pCDNA6/V5-HisA.

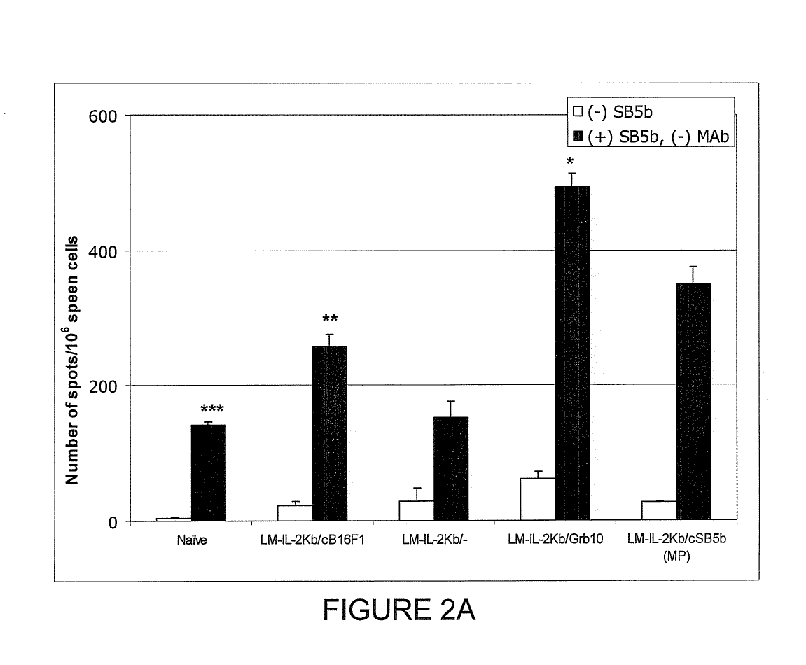

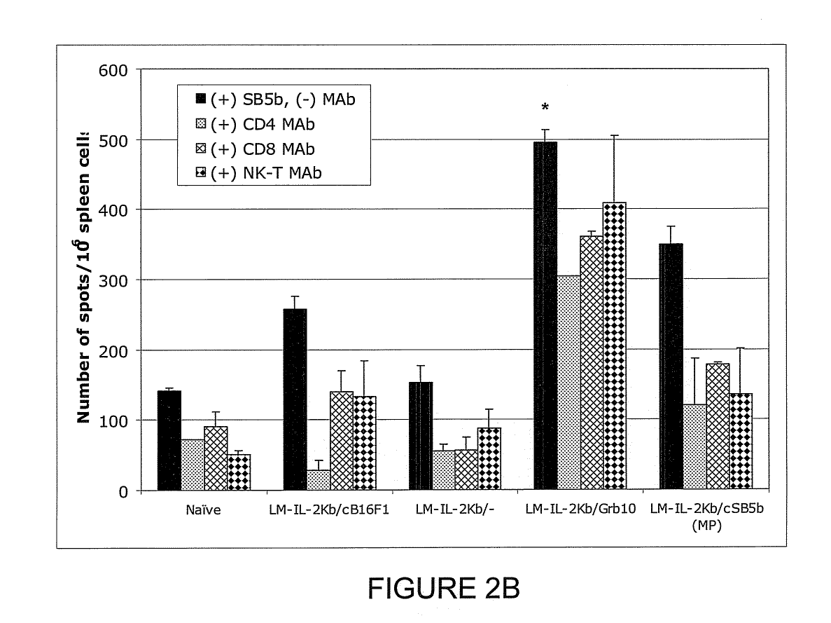

ELISPOT IFN-γ assays were used to compare the number of responding T cells in C3H/He mice immunized with LM-IL-2K^{b}/Grb10 cells with that of mice immunized with cells from various control groups. Naïve mice were injected s.c. two times at weekly intervals with 4×10⁶ LM-IL-2K^{b}/Grb10 cells in each injection. One week after the last injection, spleen cells from the immunized mice were co-incubated for 18 hrs in the presence or absence of mitomycin C-treated SB5b cells (Effector:Target cell [E:T] ratio=10:1). At the end of the incubation, the number of responding T cells was determined in a standard ELISPOT IFN-γ assay. As controls, the same protocol was followed except the mice were injected with an equivalent number of cells from the master pool (non-enriched), with non-transduced fibroblasts (LM-IL-2K^{b}/−) or, as a specificity control, with fibroblasts transduced with a cDNA library derived from B16F1 cells (LM-IL-2K^{b}/cB16F1). One group of mice was not injected (naive). These results are shown in FIG. 2A (*, P<0.0002 for the number of spots/10⁶ spleen cells in cultures of spleen cells from mice immunized with LM-IL-2K^{b}/Grb10 cells co-incubated with SB5b cells versus the number of spots/10⁶ spleen cells incubated alone; **, P<0.001 for differences in the number of spots/10⁶ spleen cells in cultures of cells from mice immunized with LM-IL-2K^{b}/Grb10 cells co-cultured with SB5b cells relative to the number of spots in spleen cell cultures from mice immunized with LM-IL-2K^{b}/cB16F1 cells co-cultured with SB5b cells; ***, P<0.001 for differences in the number of spots/10⁶ spleen cells in cultures of cells from mice immunized with LM-IL-2K^{b}/Grb10 cells co-cultured with SB5b cells relative to the number of spots in cultures from mice immunized LM-IL-2K^{b}/− cells co cultured with SB5b cells. Pooled spleen cells were from three mice in each group.)

As indicated in FIG. 2A, the highest number of responding cells (number of spots/10⁶ spleen cells) was in the group immunized with LM-IL-2K^{b}/Grb10 cells. Lesser numbers of responding cells were detected if the spleen cells were from mice immunized with cells from the master pool (non-enriched) or from mice immunized with non-transduced fibroblasts, or from mice immunized with LM-IL-2K^{b}/CB16F1 cells. The number of responding T cells in mice immunized with LM-IL-2K^{b}/cB16F1 cells was not significantly different than that of mice immunized with cells from the non-enriched master pool. Significantly lesser responses were obtained if the mice were immunized with LM-IL-2K^{b}/− cells (non-transduced) or if the spleen cells were from naïve mice (p<0.001).

The types of cells activated for immunity to the breast cancer cells in mice immunized with LM-IL-2K^{b}/Grb10 cells was investigated by determining the effect of CD4⁺, CD8⁺and NK1.1 mAbs on the number of responding cells. Monoclonal antibodies (mAbs) for CD4⁺, CD8⁺and NK1.1 cells and fluorescein-conjugated mAbs for H-2K^{b} class I-determinants were from B-D Pharmingen (San Jose, Calif.). For these studies, the same protocol used to generate the data in FIG. 2A (described above) was followed, except that mAbs were added 1 hr and complement was added 30 min before the ELISPOT IFN-γ assays were performed. As indicated in FIG. 2B, each of the mAbs inhibited the response (*, P<0.05 for the difference in the number of spots in the presence or absence of CD4 or CD8 mAb in cocultures of spleen cells from mice injected with LM-IL-2K^{b}/Grb10 cells and mitomycin C-treated SB5b cells). CD4⁺ mAbs inhibited the response to the greatest extent. Analogous effects were obtained if the mice were immunized with cells from the non-enriched master pool (LM-IL-2K^{b}/cSB5b) or with LM-IL-2K^{b}/cB16F1 cells.

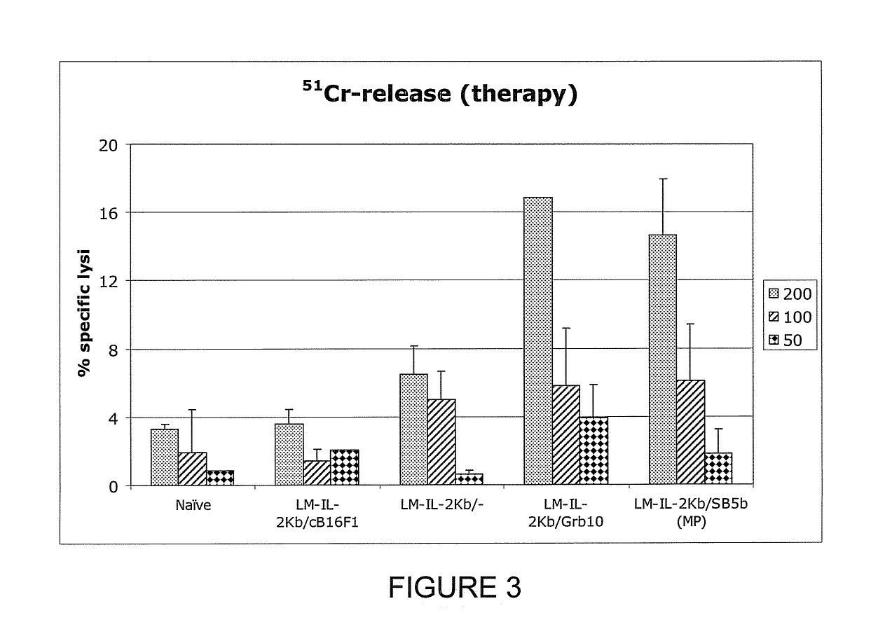

⁵¹Cr-release cytotoxicity assays were used to measure CTL responses toward SB5b cells in mice immunized with LM-IL-2K^{b}/Grb10 cells. To establish tumors, naïve C3H/He mice received one s.c. injection of 0.4×10⁶ viable SB5b cells. One week later, when the tumors were approximately 3 mm in diameter, the mice received the first of two s.c. injections at weekly intervals of 4×10⁶ LM-IL-2K^{b}/Grb10 cells. As controls, the same procedure was followed except that the tumor-bearing mice were immunized with cells from the master pool (LM-IL-2K^{b}/cSB5b), with LM-IL-2K^{b}/− cells (non-transduced), or with LM-IL-2K^{b}/cB16F1 cells. One group of mice was not treated (naïve). One week after the second injection, spleen cells from the immunized mice were co-cultured for 5 days with mitomycin C-treated SB5b cells (ratio of spleen cells to SB5b cells=30:1). After incubation, the non-adherent cells were collected and co-incubated for 5 hrs with ⁵¹Cr-labeled SB5b cells before the specific cytotoxicity was determined. Approximately 15% of maximum ⁵¹Cr release was released spontaneously (background). The results are shown in FIG. 3, where P<0.03 for the specific release of isotope above background from SB5b cells co-incubated with spleen cells from mice immunized with LM-IL-2K^{b}/Grb10 cells versus that of SB5b cells co-incubated with spleen cells from non-treated tumor-bearing mice. Pooled spleen cells were from three mice in each group.

The results shown in FIG. 3 indicated that the highest responses (percent specific lysis of SB5b cells) were in mice immunized with LM-IL-2K^{b}/Grb10 cells. Equivalent responses were detected in mice immunized with cells from the non-enriched master pool (LM-IL-2K^{b}/cSB5b). Lesser responses were present in mice immunized with LM-IL-2K^{b}/− cells. The response in tumor-bearing mice immunized with LM-IL-2/cB16F1 cells was not significantly different than that of untreated tumor-bearing mice.

Thus, augmented immunity to the breast cancer cells was generated in mice immunized with the fibroblasts modified to express the gene specifying Grb10, as determined by two independent assays.

### Example 5

### Survival of C3H/He Mice with Breast Cancer Immunized with LM-IL-2K^{b}/Grb10 Cells

Enhanced T-cell immunity toward SB5b cells was generated in mice immunized with modified fibroblasts transduced with the gene for Grb10. To determine if the immunogenic properties of the vaccine observed in vitro were reflected by the cells' immunotherapeutic properties in mice with breast cancer, tumors were first established in C3H/He mice injected with 0.4×10⁶ SB5b cells, followed by the first of two weekly injections of 4×10⁶ LM-IL-2K^{b}/Grb10 cells 7 days later. The size of the tumor at the injection site was 5±2 mm at the time of the first injection. As controls, the same procedure was followed except that LM-IL-2K^{b}/cSB5b cells, non-transduced LM-IL-2Kb/− cells, or cells transduced with cDNA from B16F1 melanoma cells were substituted for LM-IL-2K^{b}/Grb10 cells. One group of tumor-bearing mice was not treated (naïve). The surviving mice were euthanized 50 days after injection of the breast cancer cells.

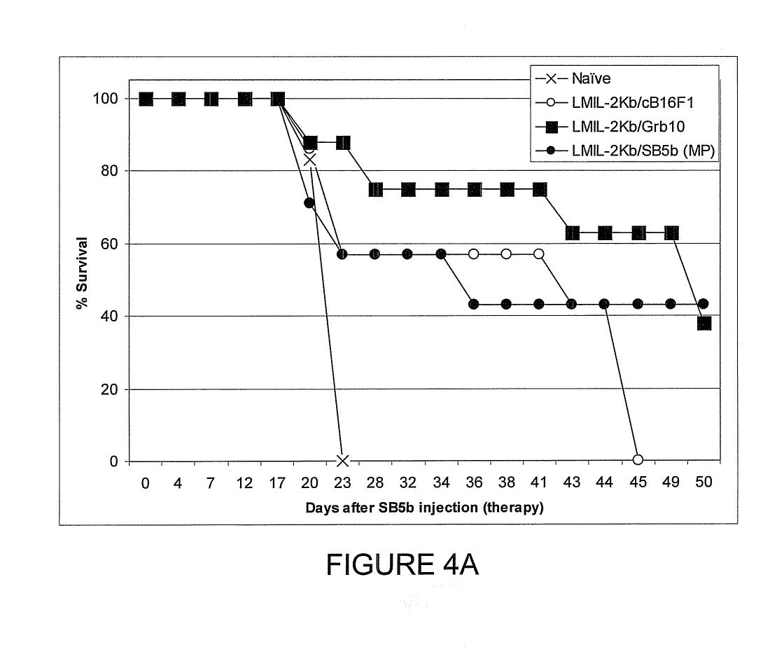

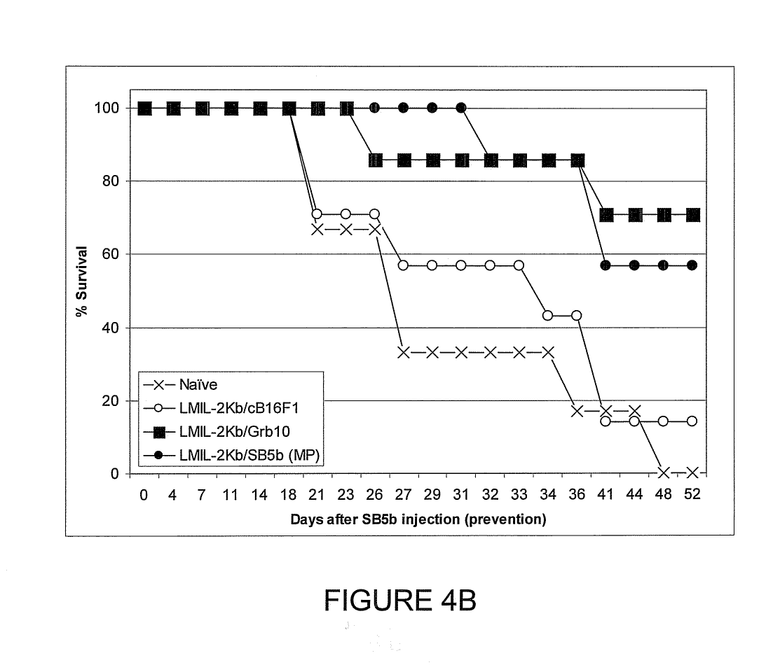

Results are shown in FIG. 4A. Mean survival time (MST) for mice injected with SB5b cells followed by the injections of LM-IL-2K^{b}/Grb10 cells was 41.0±11.0 days; MST for mice injected with SB5b cells followed by the injections of non-enriched LM-IL-2K^{b}/cSB5b (master pool) cells was 40.0±14.0 days; MST for mice injected with SB5b cells followed by the injections of LM-IL-2K^{b}/cB16F1 cells was 34.0±12.0 days; and MST for naïve mice injected with SB5b cells alone was 22.0±1.3 days. P<0.01 for survival of mice with breast cancer treated by immunization with LM-IL-2K^{b}/Grb10 cells versus naïve mice. P<0.02 for survival of mice with breast cancer treated by immunization with LM-IL-2K^{b}/Grb10 cells versus mice with breast cancer treated by immunization with LM-IL-2K^{b}/cB16F1 cells. There were eight mice in each group.

Kaplan-Meier log rank analyses were used to determine the statistical differences between the survival of mice in the various experimental and control groups. A p value less than 0.05 was considered significant. Student t test one-way Anova was used to determine the statistical differences between experimental and control groups in the experiments performed in vitro.

As indicated in FIG. 4A, tumor-bearing mice treated by immunization with LM-IL-2K^{b}/Grb10 cells survived significantly (P<0.01) longer than mice in any of the control groups, including mice immunized with LM-IL-2K^{b}/cB16F1 cells, except mice treated by immunization with LM-IL-2K^{b}/cSB5b cells. Although differences in the survival of treated and untreated mice with breast cancer were highly significant, the immunotherapeutic properties of LM-IL-2K^{b}/cSB5b cells and LM-IL-2K^{b}/Grb10 cells were not statistically different.

In a separate study, tumor-free mice were immunized with LM-IL-2K^{b}/Grb10 cells before injection of the breast cancer cells. C3H/He mice received two s.c. injections at weekly intervals of 4×10⁶ LM-IL-2K^{b}/Grb10 cells. One week after the last injection, the mice were injected s.c. with 0.4×10⁶ SB5b cells. As controls, the same procedure was followed except that equivalent numbers of LM-IL-2K^{b}/− cells or non-enriched cells from the master pool or LM-IL-2K^{b}/cB16F1 cells were substituted for LM-IL-2K^{b}/Grb10 cells. One group of mice was injected with SB5b cells alone (naïve mice). The experiment was terminated 52 days after injection of the breast cancer cells.

These results are shown in FIG. 4B. MST for mice injected with LM-IL-2K^{b}/Grb10 cells followed by the injection of SB5b cells was 47.0±10.0 days; MST for mice injected with cells from the master pool followed by the injection of SB5b cells was 45.0±9.1 days; MST for mice injected with LM-IL-2K^{b}/cB16F1 cells was 34.0±11.0 days; and MST for naïve mice injected with SB5b cells alone was 29.0±9.0 days. P<0.001 for survival of mice immunized with LM-IL-2K^{b}/Grb10 cells or cells from the master pool versus naïve mice. P<0.01 for survival of mice immunized with LM-IL-2K^{b}/Grb10 cells or cells from the master pool and mice immunized with LM-IL-2K^{b}/cB16F1 cells. There were seven mice in each group.

As indicated in FIG. 4B, immunization with LM-IL-2K^{b}/Grb10 cells prevented the growth and prolonged the survival of tumor-free C3H/He mice that were later injected with SB5b cells.

It should be understood that the foregoing disclosure emphasizes certain specific embodiments of the invention and that all modifications or alternatives equivalent thereto are within the spirit and scope of the invention as set forth in the appended claims.

1. An isolated mammalian cell expressing a tumor-associated antigen, wherein administration of the isolated mammalian cell to a mammal induces an immune response to the tumor-associated antigen in the mammal, and wherein the tumor-associated antigen is Grb10, Triple motif protein 13, Serum amyloid A3, Xlr related meiosis regulated protein. Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product.

2. The isolated mammalian cell according to claim 1, wherein the tumor-associated antigen is Grb10.

3. The isolated mammalian cell according to claim 1, wherein the isolated mammalian cell is allogeneic to the mammal.

4. The isolated mammalian cell according to claim 1 further comprising a recombinant construct encoding exogenous interleukin 2 (IL2), Granulocyte-macrophage colony-stimulating factor (GMCSF) or IL17.

5. The isolated mammalian cell according to claim 1, wherein the mammal is a human.

6. The isolated mammalian cell according to claim 1 adapted for administration to a mammal in a formulation comprising a pharmaceutically acceptable carrier, adjuvant, or diluent.

7. A therapeutic tumor vaccine comprising the isolated mammalian cell according to claim 1 and a pharmaceutically acceptable carrier, diluent or adjuvant.

8. The therapeutic tumor vaccine of claim 7, wherein the tumor-associated antigen is Grb10.

9. The therapeutic tumor vaccine of claim 7 wherein the isolated mammalian cell is allogeneic to the mammal.

10. The therapeutic tumor vaccine of claim 7, wherein the isolated mammalian cell further comprises a recombinant construct encoding exogenous interleukin 2 (IL2), Granulocyte-macrophage colony-stimulating factor (GMCSF) or IL17.

11. The therapeutic tumor vaccine of claim 7, further comprising an immune stimulatory agent.

12. A pharmaceutical composition comprising an isolated mammalian cell that expresses a tumor-associated antigen in an amount effective to inhibit proliferation of a target cell in a mammal, and a pharmaceutically acceptable carrier, adjuvant, or diluent, wherein the tumor-associated antigen is Grb10, Triple motif protein 13, Serum amyloid A3, Xlr related meiosis regulated protein, Pentaxin related gene, CD36 antigen, RIKEN cDNA 9030625A04 gene, Prostaglandin-endoperoxide synthase 2, RIKEN cDNA E030003E18 gene, Tumor-associated calcium signal transducer 1, RIKEN cDNA 2310005E10 gene, DEAD (Asp-Glu-Ala-Asp) box polypeptide 25, Neuropeptide Y receptor Y1, GRP1 (general receptor for phosphoinositides 1)-associated scaffold protein, Nuclear receptor subfamily 4 group A member 1, SRY-box containing gene 5, Carbonic anhydrase 9, Aldehyde dehydrogenase family 1 subfamily A7, Thymoma viral proto-oncogene 3 or RIKEN cDNA D130020G16 gene product.

13. The pharmaceutical composition of claim 12, wherein the tumor-associated antigen is Grb10.

14. The pharmaceutical composition of claim 12 wherein the isolated mammalian cell is allogeneic to the mammal.

15. The pharmaceutical composition of claim 12, wherein the isolated mammalian cell further comprises a recombinant construct encoding exogenous interleukin 2 (IL2), Granulocyte-macrophage colony-stimulating factor (GMCSF) or IL17.

16. The pharmaceutical composition of claim 12, further comprising an immune stimulatory agent.

17. A method of inhibiting proliferation of a target cell in a mammal comprising administering to the mammal an effective amount of the pharmaceutical composition according to claim 12, wherein proliferation of the target cell is inhibited thereby.

18. The method of claim 17, wherein the proliferation of the target cell is inhibited by an immune response to the tumor-associated antigen induced after administration of the pharmaceutical composition to the mammal.

19. The method of claim 18, wherein the immune response is CD4 cell, CD8 cell, or NK cell-mediated immune response.

20. The method of claim 18, wherein the target cell expresses the tumor-associated antigen.

21. The method of claim 20, wherein the tumor-associated antigen is Grb10.

22. The method of claim 21, wherein the target cell is a tumor cell.

23. The method of claim 22 wherein the tumor cell is melanoma, lymphoma, plasmacytoma, sarcoma, glioma, thymoma, leukemia, breast cancer, prostate cancer, colon cancer, esophageal cancer, brain cancer, lung cancer, ovarian cancer, cervical cancer or hepatoma cell.

24. The method of claim 23, wherein the tumor cell is a breast cancer cell.

25. The method of claim 17, wherein the mammal is a human.

26. A method of inhibiting proliferation of a target cell comprising administering to a mammal an effective amount of an isolated mammalian cell according to claim 1, wherein the proliferation of the target cell is inhibited thereby.

27. The method of claim 26, wherein the tumor-associated antigen is Grb10.

28. The method of claim 26, wherein the isolated mammalian cell is allogeneic to the mammal.

29. The method of claim 26, wherein the isolated mammalian cell further comprises a recombinant construct encoding exogenous interleukin 2 (IL2), Granulocyte-macrophage colony-stimulating factor (GMCSF) or IL17.

30. The method of claim 26, further comprising administering to the mammal an immune-stimulating agent.

31. The method of claim 29 wherein the proliferation of the target cell is inhibited by an immune response to the tumor-associated antigen induced after administration of the effective amount of the isolated mammalian cell to the mammal.

32. The method of claim 31, wherein the immune response is CD4 cell, CD8 cell, or NK cell-mediated immune response.

33. The method of claim 31 wherein the target cell expresses Grb10.

34. The method of claim 33, wherein the target cell is a tumor cell.

35. The method of claim 34 wherein the tumor cell is melanoma, lymphoma, plasmacytoma, sarcoma, glioma, thymoma, leukemias, breast cancer, prostate cancer, colon cancer, esophageal cancer, brain cancer, lung cancer, ovarian cancer, cervical cancer or hepatoma cell.

36. The method of claim 35, wherein the tumor cell is a breast cancer cell.

37. The method of claim 26 wherein the mammal is a human.

38. A method of inhibiting tumor growth in a mammal comprising administering to a mammal an effective amount of an isolated mammalian cell according to claim 1, wherein the tumor growth in the mammal is inhibited thereby.

39. The method of claim 38 wherein the tumor-associated antigen is Grb10.

40. The method of claim 38 wherein the isolated mammalian cell is allogeneic to the mammal.

41. The method of claim 38, wherein the isolated mammalian cell further comprises a recombinant construct encoding exogenous interleukin 2 (IL2), Granulocyte-macrophage colony-stimulating factor (GMCSF) or IL17.

42. The method of claim 38, further comprising administering to the mammal an immune-stimulating agent.

43. The method of claim 38 wherein an immune response to the tumor-associated antigen is induced in the mammal after administration of the effective amount of the isolated mammalian cell to the mammal.

44. The method of claim 43, wherein the tumor-associated antigen is Grb10.

45. The method of claim 43, wherein the immune response is a CD4 cell, CD8 cell, or NK cell-mediated immune response.

46. The method of claim 43, wherein the tumor expresses Grb10.

47. The method of claim 46, wherein the tumor is breast tumor.

48. The method of claim 38, wherein the mammal is a human.

49. A method of enhancing an immune response to a tumor-associated antigen in a mammal comprising administering to a mammal an effect amount of an isolated mammalian cell according to claim 1.

50. The method of claim 49, wherein the tumor-associated antigen is Grb10.

51. The method of claim 49, wherein the isolated mammalian cell is allogeneic to the mammal.


SEQUENCE LISTING (87 sequences)

SEQ ID NO: 1 - DNA, 4799 bp, Mus musculus
  CDS (175)..(1800)
atcgccatctacagtttctgtctgttagaggagagtgtgaaatctactgcgtcctagctctgtaccttggacaaagtggggcagtcaacgtccaaacccgaaaacctagctaagtctgggttttcgccacaacaaagaagccaaccagagcatggtcttgggcttcaagtactaatgaacaacgatattaactcgtccgtggaaagccttaactcagcttgcaacatgcagtctgatactgatactgcaccacttcttgaggatggccagcatgccagcaaccagggagcagcatctagctcccggggacagccacaggcgtccccgaggcagaaaatgcaacgctcgcagcctgtgcacattctcaggcgccttcaggaggaagaccagcagttaagaactgcatctcttccggccatccccaacccatttccggagctcactggtgcggcccctgggagccctccttcggttgctcctagctccttacctcctcctccgagccagccacctgccaagcatgatgtcaaagtctttagtgaagatgggaccagcaaagtggtggagattctaaccgacatgacagccagggacctgtgccagctgctggtttacaaaagtcactgtgtggatgacaacagctggactctggtggaacaccacccacaactgggattagagaggtgcctggaggaccatgagatcgtggtccaagtggagagtaccatgccaagtgagagcaaattcttattcagaaagaattatgcgaagtacgagttctttaagaatccagtgaacttcttcccggatcagatggtcaattggtgccagcagtccaacggtggccaggcgcagcttctgcagaattttctgaacaccagcagctgccctgagatccaggggttcttgcaggtgaaagaggtaggacgcaagtcttggaagaagctgtatgtgtgcctgcgcagatctggcctctattactccaccaaggggacttcaaaagaacccagacacctgcagctgctggctgacctggaagaaagcagcatcttctacctgattgctggaaagaagcagtacaacgcgccgaatgaacatgggatgtgcatcaagccaaacaaagcgaagaccgagatgaaggagcttcgtctgctctgtgccgaagatgagcagatccgtacttgctggatgactgccttcagactgctcaagtacggaatgctcctgtaccaaaactatcgcatcccacagaggaagggtctgccccctcctttcaacgcacctatgcgcagtgtttctgagaattctcttgtggccatggatttttctggacaaatcggaagagtgatcgataacccggctgaagcccagagtgctgccctggaagagggccatgcctggcgtaagcggagcacacggatgaatatcctaagcagccaaagcccactgcatccttctaccctgaatgcagtgattcacaggactcagcattggttccatggacgtatctcccgcgaggagtctcacaggatcatcaagcaacaaggtctcgtggacgggctgttcctccttcgtgacagccagagtaatccaaaggcgttcgtactgacactgtgccatcaccagaagattaaaaacttccagatcttaccttgcgaggatgatgggcagaccttcttcactctggatgatgggaacaccaagttctccgatctgatccagctggtcgacttctaccagctcaacaaaggtgttctgccctgcaagctgaaacaccactgcatccgcgtggccttatgacctccttgcccactcacagaggctggaggcagcgacactggaacggagaagagagatctgcatgagggtgagaacacacacctactcccacccaaggactcagaacaacatggctttttgatcggtaccaaccgacctacatcaattagttattggacttcacaaagatttgccgctgtggatcaaacaggaccacctccctctgcgtcagccatttaaaattgggggagggaagagatccagtgcaagtgtgggaagaaactggaatgacgattttgattaggccacgtagggtgaaaaccgcagaaaaatgattggaaatgaactcttgccctggaataaccttgacaattcaaaccgcgatgtttactttttgtattgatcacttttttgcactccttcttcgttgtcgatgttgtactcagcctatggtaggagaggatgtggctttgcaactcagatcagacaaagaatttttgaattggcaggctttagcctacaaatgggtcccccaagttttctagataacatcctgctctctgcaggttgcggcagtgtgggtttggctccagtccttcccattatacttgaaaagcttttttttttttttaaataaaataataagataaaaggtgacaaccgcagttgacacctagcatgttttgtggacgcagtcaagctgccacagtctggtcatcgttgttgttgtgctgacgtctggtcatgtattattagtgtgtctcgttctccacggtgctggagacctcattactttggaaaactcactgttccccagcacagcacagctgcatgacctcagcttcagactgatgtcagccagccttcttggaacacattgctattcattgtggatgccacacagtccatctggggtgccctgtgcctcccattttcacagccgcccactgttgtagaataatcgctgcctttaccctctgcagtaccctcagtcatttcacttctcccacaggggtggggtaggggtggggttaaaggatcgaaagagaaaaaaacgccaacttgttggccttgtgctctgtcactctgagcatctatggtgataaagagaaggaaacgctgcagaggcacaagcataagctggctttgagtctttgaaagcttgatggcctctgcaccctactgaaaaccccctaagccagcaggagcagggtatgcagaggctttgcctctgctggcttagggtgagaagtacctcacaatggatgtgaacattggacagattttaggagcatgatgtttgcctcctccgagagagaggagtcagagagagagaggagtcagaaggtggccggagttatcattgggtcccctgagctccatgaaggaccctctgttgtccccttgatgatttatcaggcatgagagtagcagacaggattgccttgagaaacacacagacaacaacggtggtgaacacatttccctttcttgataccacccagaaagtctgctacatacctgagttagccactagattgcctgtcccgactcattgaagtgctccccagggagccacttctgatttgccatggttgacagcatagtggaagatagatatgacagcgctttgtaaagcaggccagtggcaagctggcccacagtagagaaacactgtagttcacagaccatgcaaccgtgtttccacgagatgttattacaacaaatgaagaatttttttccttttttttcattttaatcttttttgacttttttttagttaaccattttttatgcataagtgctcaaatgcatttctctttctttcgaaactagttaatcgttaagttgacaatgagagaattctcaagttcatacctggcagcaggctcccactgatcttccccttacagacagataacagacatttcagttttaccgtgctcatttctttttgctgacttaaggtcagaacttttacagacaacaaacaaccctagggtttctttttccagtttacacagaccggtccccacagtgcagaatccatttctctttcgtctcaacagtagacaactaggatgtggatctcatatttataagtatgcattttatttaagaggaagtataggcttgactctggttcacaatttcgtacgtagctggtttgacgtagaactttgtacttcccttgccgaagtgaattgttgaaggctgcaacccacccaccttgagtgtagcagacttcagtggccccgagatcgccagccctttgcacaggcagctgggaattccacctgaaacagctggtccctaggttagcgggttcccagcccccctaatcagagactgaaggacaattgacttttatttttgtatttaattgacatgaatgtaaaggggacagctcagggttgttttggagcctgtttgactttgtaatctctgcctgtgattttcttttctaaatgacgactccgtgtaaccacctggactaagttgagaaggaaactgccaaatgctttgggtttttagggttttaataggtagactctgttctattattaggtgttaagagtttccaaacgtgttttcttttttcttttattgtttgcttaaactttgaagaaatatgtgcctcagcttagatgttttgtcttcccctttctgcacttaaatacctgacagcctgttcgatcgctgtgcctccgagggcgcttctagctcatcgtagatttgtgatgtcatagtgcaaactgcagtgaccggtaaaatgacctgacatgtaaccgttttcagggaatgcagagggtgttaactaatagacaaaacctttatcccgcgtgctttgcttcaccttgtgctatatagctctttggctcgtgaacctaattgtaaactttcaggtatttttgtacaaataagggactgatgttctgtttcttgttattagaaataaacattaataaagcgttaaaaaaaaaaaaaaaaaaaaaaaaaaaaaaaaaaaaaaaaaaaaa

SEQ ID NO: 2 - protein, 541 aa, Mus musculus
MNNDINSSVESLNSACNMQSDTDTAPLLEDGQHASNQGAASSSRGQPQASPRQKMQRSQPVHILRRLQEEDQQLRTASLPAIPNPFPELTGAAPGSPPSVAPSSLPPPPSQPPAKHDVKVFSEDGTSKVVEILTDMTARDLCQLLVYKSHCVDDNSWTLVEHHPQLGLERCLEDHEIVVQVESTMPSESKFLFRKNYAKYEFFKNPVNFFPDQMVNWCQQSNGGQAQLLQNFLNTSSCPEIQGFLQVKEVGRKSWKKLYVCLRRSGLYYSTKGTSKEPRHLQLLADLEESSIFYLIAGKKQYNAPNEHGMCIKPNKAKTEMKELRLLCAEDEQIRTCWMTAFRLLKYGMLLYQNYRIPQRKGLPPPFNAPMRSVSENSLVAMDFSGQIGRVIDNPAEAQSAALEEGHAWRKRSTRMNILSSQSPLHPSTLNAVIHRTQHWFHGRISREESHRIIKQQGLVDGLFLLRDSQSNPKAFVLTLCHHQKIKNFQILPCEDDGQTFFTLDDGNTKFSDLIQLVDFYQLNKGVLPCKLKHHCIRVAL

SEQ ID NO: 3 - DNA, 2312 bp, Homo sapiens
  CDS (310)..(1920)
gagccgcgcgcggcagctttggcgctgaccacaatgctgagcaggaagcagcagctgcaggcccagtgactggtagctcagtgaccagcagcccagtgaccggcagccaggtcctcacctgggtcctctcagtgaagccagggtggccgccccagcagacagtgctacagagccaactcctgacaggacaaggtggagcagacacctcgcagtcaacaagacccggcaggaccaggactccccgcacagtctgaccgacttgcgaatcaccaggaggatgatgtggacctggaagccctggtgaacgatatgaatgcatccctggagagcctgtactcggcctgcagcatgcagtcagacacggtgcccctcctgcagaatggccagcatgcccgcagccagcctcgggcttcaggccctcctcggtccatccagccacaggtgtccccaaggcagagggtgcagcgctcccagcctgtgcacatcctcgctgtcaggcgccttcaggaggaagaccagcagtttagaacctcatctctgccggccatccccaatccttttcctgaactctgtggccctgggagcccccctgtgctcacgccgggttctttacctccgagccaggccgccgcaaagcaggatgttaaagtctttagtgaagatgggacaagcaaagtggtggagattctagcagacatgacagccagagacctgtgccaattgctggtttacaaaagtcactgtgtggatgacaacagctggacactagtggagcaccacccgcacctaggattagagaggtgcttggaagaccatgagctggtggtccaggtggagagtaccatggccagtgagagtaaatttctattcaggaagaattacgcaaaatacgagttctttaaaaatcccatgaatttcttcccagaacagatggttacttggtgccagcagtcaaatggcagtcaaacccagcttttgcagaattttctgaactccagtagttgtcctgaaattcaagggtttttgcatgtgaaagagctgggaaagaaatcatggaaaaagctgtatgtgtgtttgcggagatctggcctttattgctccaccaagggaacttcaaaggaacccagacacctgcagctgctggccgacctggaggacagcaacatcttctccctgatcgctggcaggaagcagtacaacgcccctacagaccacgggctctgcataaagccaaacaaagtcaggaatgaaactaaagagctgaggttgctctgtgcagaggacgagcaaaccaggacgtgctggatgacagcgttcagactcctcaagtatggaatgctcctttaccagaattaccgaatccctcagcagaggaaggccttgctgtccccgttctcgacgccagtgcgcagtgtctccgagaactccctcgtggcaatggatttttctgggcaaacaggacgcgtgatagagaatccggcggaggcccagagcgcagccctggaggagggccacgcctggaggaagcgaagcacacggatgaacatcctaggtagccaaagtcccctccacccttctaccctaagtacagtgattcacaggacacagcactggtttcacgggaggatctccagggaggaatcccacaggatcattaaacagcaagggctcgtggatgggctttttctcctccgtgacagccagagtaatccaaaggcatttgtactcacactgtgtcatcaccagaaaattaaaaatttccagatcttaccttgcgaggacgacgggcagacgttcttcagcctagatgacgggaacaccaaattctctgacctgatccagctggttgacttttaccagctgaacaaaggagtcctgccttgcaaactcaagcaccactgcatccgagtggccttatgaccgcagatgtcctctcggctgaagactggaggaagtgaacactggagtgaagaagcggtctgtgcgttggtgaagaacacacatcgattctgcacctggggacccagagcgagatgggtttgttcggtgccagccgaccaagattgactagtttgttggacttaaacgacgatttgctgctgtgaacccagcagggtcgcctccctctgcgtcggccaaattggggagggcatggaagatccagcggaaagttgaaaataaactggaatgatcatcttggcttgggccgcttaggaacaagaaccggagagaagtgattggaaatgaactcttgccctggaataatcttgacaattaaaactgatatgtttaaaaaaaaaaaaaaaaaaaaa

SEQ ID NO: 4 - protein, 536 aa, Homo sapiens
MNASLESLYSACSMQSDTVPLLQNGQHARSQPRASGPPRSIQPQVSPRQRVQRSQPVHILAVRRLQEEDQQFRTSSLPAIPNPFPELCGPGSPPVLTPGSLPPSQAAAKQDVKVFSEDGTSKVVEILADMTARDLCQLLVYKSHCVDDNSWTLVEHHPHLGLERCLEDHELVVQVESTMASESKFLFRKNYAKYEFFKNPMNFFPEQMVTWCQQSNGSQTQLLQNFLNSSSCPEIQGFLHVKELGKKSWKKLYVCLRRSGLYCSTKGTSKEPRHLQLLADLEDSNIFSLIAGRKQYNAPTDHGLCIKPNKVRNETKELRLLCAEDEQTRTCWMTAFRLLKYGMLLYQNYRIPQQRKALLSPFSTPVRSVSENSLVAMDFSGQTGRVIENPAEAQSAALEEGHAWRKRSTRMNILGSQSPLHPSTLSTVIHRTQHWFHGRISREESHRIIKQQGLVDGLFLLRDSQSNPKAFVLTLCHHQKIKNFQILPCEDDGQTFFSLDDGNTKFSDLIQLVDFYQLNKGVLPCKLKHHCIRVAL

SEQ ID NO: 5 - DNA, 19 bp, Artificial Sequence
  Synthetic PCR primer - Grb10 forward
cgtggtccagtggagagta

SEQ ID NO: 6 - DNA, 21 bp, Artificial Sequence
  Synthetic PCR primer - Grb10 backward
tccggtcttcggcgtaactga

SEQ ID NO: 7 - DNA, 1360 bp, Mus musculus
  CDS (94)..(1317)
ccgtcaaaacgtgagggaaaacgaactggctctctccacagaaaatatagtaagcttgccaaaagcatatacttgcctggaacaaaggatgtaatggagctgcttgaagaagacctcacatgcccaatttgctgcagtttgtttgatgacccccgagtgttgccctgctcacacaacttctgcaaaaaatgcttagaagggctcttagaggggaatgtgcggaattccctgtggagaccatctcccttcaagtgtcctacctgccgtaaggaaacctcagctactggagtcaacagtctgcaggtcaattactccctaaagggtatcgtggagaaatacaacaaaatcaagatttctcccaagatgccagtgtgcaaaggacatttggggcagcctctcaacatcttctgcgtaactgatatgcagctgatttgtgggatctgtgctactcgaggcgagcacaccaagcatgtcttctcttctattgaagatgcctacgctcgagaaaagaatgcctttgagtccctctttcagagtttcgagacttggcgccggggagatgctctttcccgcttggatactttggaaacaaacaagaggaaagccctccagttactcacgaaggattcagataaagtaaaggagttttttgagaagttacagcacaccttggatcaaaagaagaatgaaatcctgtctgactttgaaactatgaagcttgcagttatgcaaacctatgacccggagatcaacaaaatcaacactattttacaggagcagcggatggccttcaacattgctgaggctttcaaagatgtctcagaacctattatatttttgcaacagatgcaagagttcagggagaaaatcaaagtaatcaaggaaactcctttgccacactctaatttgcccacaagccctttaatgaagaactttgataccagtcagtggggagacattaaactagttgatgtggataaactgtctttgccgcaagacacaggtgtgttcactagcaagattccctggtacccctatctgctgctcatgatggtagttctgctgggtctcctcatattctttggccccactgtattcctggaatggtctccacttgatgaattggcaacttggaaagactatctttcaagcttcaattcttacctgactaagtctgctgattttatagaacaatctgttttttactgggaacagatgacagatgggtttttcatttttggtgaaagagtaaaaaatgttagtttggtggcactgaacaatgtggcagagtttatatgcaaatacaaactattataaagtctttcaattacacagttctgcttcttgatgggattgttgg

SEQ ID NO: 8 - protein, 407 aa, Mus musculus
MELLEEDLTCPICCSLFDDPRVLPCSHNFCKKCLEGLLEGNVRNSLWRPSPFKCPTCRKETSATGVNSLQVNYSLKGIVEKYNKIKISPKMPVCKGHLGQPLNIFCVTDMQLICGICATRGEHTKHVFSSIEDAYAREKNAFESLFQSFETWRRGDALSRLDTLETNKRKALQLLTKDSDKVKEFFEKLQHTLDQKKNEILSDFETMKLAVMQTYDPEINKINTILQEQRMAFNIAEAFKDVSEPIIFLQQMQEFREKIKVIKETPLPHSNLPTSPLMKNFDTSQWGDIKLVDVDKLSLPQDTGVFTSKIPWYPYLLLMMVVLLGLLIFFGPTVFLEWSPLDELATWKDYLSSFNSYLTKSADFIEQSVFYWEQMTDGFFIFGERVKNVSLVALNNVAEFICKYKLL

SEQ ID NO: 9 - DNA, 1975 bp, Homo sapiens
  CDS (706)..(1929)
ggcgcgtaccgtgcggtccctgtagttggaggacgggcggtcgcgcggcctttcccactagccggaggtcggagataagtacccgccgcccggctcctctcgggaaagcggggtggtcctcgaaccttcagcgagggtggggagttgcccaggtcagcagggatctgcgtgggttgggggggctggcgaaggccgtctgagctccagtccggcagcgcggcagaaaccagcggggcactgtcatgggcctggggaggagcggcctgcggagggcgccggggcagtgtgtctgtggtccagaaaacctgctccgtccggagccttcctggccccgctaccagcgtctccacatcccctagaaaagaaaagacgggtgtgggccttaggttacagcgcgccgccagcgtttggttgcatggcgccgggggagggcgccctaaccgagaagctgcttaatacaaagagctccaggctcctggcggttcaccaggtctaaacagccgggctttatttgtgggggcgattgaaaaaattgagggtcaagattggggtgctgtgcaaataaatgcgttaatactgttctttttcttctttctttgcagtagcctctagttcgttagtcaaaacgtgaaaaaaaagacctgctttgccctgggaaatagtaaccctgccaaatacatcagcttgtaggagacagaggatgtgatggagctgcttgaagaagatctcacatgccctatttgttgtagtctgtttgatgatccacgggttttgccttgctcccacaacttctgcaaaaaatgcttagaaggtatcttagaagggagtgtgcggaattccttgtggagaccagctccattcaagtgtcctacatgccgtaaggaaacttcagctactggaattaatagcctgcaggttaattactccctgaagggtattgtggaaaagtataacaagatcaagatctctcccaaaatgccagtatgcaaaggacacttggggcagcctctcaacattttctgcctgactgatatgcagctgatttgtgggatctgtgctactcgtggggagcacaccaaacatgtcttctgttctattgaagatgcctatgctcaggaaagggatgcctttgagtccctcttccagagctttgagacctggcgtcggggagatgctctttctcgcttggataccttggaaactagtaagaggaaatccctacagttactgactaaagattcagataaagtgaaggaattttttgagaagttacaacacacactggatcaaaagaagaatgaaattctgtctgactttgagaccatgaaacttgctgttatgcaagcatatgacccagagatcaacaaactcaacaccatcttgcaggagcaacggatggcctttaacattgctgaggctttcaaagatgtgtcagaacccattgtatttctgcaacagatgcaggagtttagagagaaaatcaaagtaatcaaggaaactcctttacctccctctaatttgcctgcaagccctttaatgaagaactttgataccagtcagtgggaagacataaaactagtcgatgtggataaactttctttgcctcaagacactggcacattcattagcaagattccctggagcttttataagttatttttgctaatccttctgcttggccttgtcattgtctttggtcctaccatgttcctagaatggtcattatttgatgacctggcaacttggaaaggctgtctttcaaacttcagttcctatctgactaaaacagccgatttcatagaacaatcagttttttactgggaacaggtgacagatgggtttttcattttcaatgaaagattcaagaattttactttggtggtactgaacaatgtggcagaatttgtgtgcaaatataaactattataaaaaaaaaaaaaaaaaaaaaaaaaaaaaaaaaaaaaaaaaaaaaaaa

SEQ ID NO: 10 - protein, 407 aa, Homo sapiens
MELLEEDLTCPICCSLFDDPRVLPCSHNFCKKCLEGILEGSVRNSLWRPAPFKCPTCRKETSATGINSLQVNYSLKGIVEKYNKIKISPKMPVCKGHLGQPLNIFCLTDMQLICGICATRGEHTKHVFCSIEDAYAQERDAFESLFQSFETWRRGDALSRLDTLETSKRKSLQLLTKDSDKVKEFFEKLQHTLDQKKNEILSDFETMKLAVMQAYDPEINKLNTILQEQRMAFNIAEAFKDVSEPIVFLQQMQEFREKIKVIKETPLPPSNLPASPLMKNFDTSQWEDIKLVDVDKLSLPQDTGTFISKIPWSFYKLFLLILLLGLVIVFGPTMFLEWSLFDDLATWKGCLSNFSSYLTKTADFIEQSVFYWEQVTDGFFIFNERFKNFTLVVLNNVAEFVCKYKLL

SEQ ID NO: 11 - DNA, 555 bp, Mus musculus
  CDS (35)..(403)
tggggtcccagcaggagctcgcagcacgagcaggatgaagccttccattgccatcattctttgcatcttgatcctgggagttgacagccaaagatgggtccagttcatgaaagaagctggtcaagggtctagagacatgtggcgagcctactctgacatgaagaaagctaactggaaaaactcagacaaatacttccatgctcgggggaactatgatgctgcccggaggggtcccgggggagcctgggctgctaaagtcatcagtgatgccagagaggctgttcagaagttcacgggacatggagcagaggactcaagagctgaccagtttgccaacgagtggggccggagtggcaaagaccccaaccacttccgacctgctggcctgcctaaaagatactgagttttctcttcctgttgttccctgtcatgctgccccccgagaagaggggcaactactgggttgagatattttctaaaatctggatccctaaacatcccaatgtgctgaataaatacttgtgaaatgcaaaaaaaaaaaaaaaaaaaaaaaaa

SEQ ID NO: 12 - protein, 122 aa, Mus musculus
MKPSIAIILCILILGVDSQRWVQFMKEAGQGSRDMWRAYSDMKKANWKNSDKYFHARGNYDAARRGPGGAWAAKVISDAREAVQKFTGHGAEDSRADQFANEWGRSGKDPNHFRPAGLPKRY

SEQ ID NO: 13 - DNA, 716 bp, Homo sapiens
  CDS (224)..(592)
aggctcagtataaatagcagccaccgctccctggcaggcagggacccgcagctcagctacagcacagatcaggtgaggagcacaccaaggagtgatttttaaaacttactctgttttctctttcccaacaagattatcatttcctttaaaaaaaatagttatcctggggcatacagccataccattctgaaggtgtcttatctcctctgatctagagagcaccatgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgcctgggctgcagaagtgatcagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaact

SEQ ID NO: 14 - protein, 122 aa, Homo sapiens
MKLLTGLVFCSLVLGVSSRSFFSFLGEAFDGARDMWRAYSDMREANYIGSDKYFHARGNYDAAKRGPGGAWAAEVISDARENIQRFFGHGAEDSLADQAANEWGRSGKDPNHFRPAGLPEKY

SEQ ID NO: 15 - DNA, 924 bp, Mus musculus
  CDS (164)..(802)
caggaatctctgctgacaacggttttttaccattgaggagttgagcacggaagggtgcagttgtgaaggtgttcacctctgagataacctttaagtgataatcggctctgctcagggttgttggacagttaatcgagaagacaactgagcgcttaagagaaaaatgtctattaagaaactgtgggtgataccaaaggatggttacttattacttcttgactatgattcagatgaagaagaagagcaggctcattctgaagtaaagaggccagcatttggcaaacatgagaatatgccgcctcacgtagaagcagatgaagatataagagatgaacaagacagtatgttggataaatctggagaaaacgtaagtttctcagaggaatggcagcgttttgcacgttctgtagagacaccaatggaaaactggaatttgttaagtggtgaacagcaagttaggaatgcttcagaattggaccttatggaagtacagaatccagtaactcatgatgatgggaatgcaaatcctgaagaagtagttggagatacacgaaagaagatcaacaacaaattgtgtgagcagaagtttgatatggatatacagaaattcaatgaagaacaagaaaaatcagtgaacaattatcaaaaagaacaacaagcattaaaactgtccgaatgtagtcagagtccgaccatggaagcaattgaagacatgcatgagaagtccatggagggtttgatgaacatggagaccaacaactacgatatgctttttgatgtagatggtgaagagacattatgaagcatactctgaagtactcttctcctttcccatcttcagactaatgaagcatacatatttctcacttgctggtacatttgtaacctaataaaaaattcactaacatttttgtctcctatgaa

SEQ ID NO: 16 - protein, 212 aa, Mus musculus
MSIKKLWVIPKDGYLLLLDYDSDEEEEQAHSEVKRPAFGKHENMPPHVEADEDIRDEQDSMLDKSGENVSFSEEWQRFARSVETPMENWNLLSGEQQVRNASELDLMEVQNPVTHDDGNANPEEVVGDTRKKINNKLCEQKFDMDIQKFNEEQEKSVNNYQKEQQALKLSECSQSPTMEAIEDMHEKSMEGLMNMETNNYDMLFDVDGEETL

SEQ ID NO: 17 - DNA, 1924 bp, Mus musculus
  CDS (176)..(1321)
gggaactccctcgcgctgtgccaccgacattagtcattcatcccgctcatgctttggagcgtttattaagggcttcactcctgcctcacactatctctcccgggctcaaactcggatcactgtagagtctcgcttcttcccctgcggctgcgaagcaaatttcgcctctccagcaatgcacctccctgcgatcctgctttgtgctctctggtctgcagtagtggctgagacctcggatgactacgagctcatgtatgtgaatttggacaacgaaatagacaatggacttcatcccaccgaggaccccacgccatgcgactgccgccaggagcactcggagtgggacaagctgttcatcatgctggagaactcgcagatgcgggagggcatgctgttgcaggccaccgacgacgtcctccgtggagagctgcagcggctgcgggcagagctggggcggctggcgggcggcatggcgaggccgtgcgcagccggtggccccgcagacgccaggctggtgcgggcgctggagccgctgctgcaggagagccgtgacgcgagcctcaggctggcgcgcctggaggacgcggaggcgcggcgacccgaggcgacagtgcctggcctaggcgctgtgctggaggaactgcggcggacgcgcgccgacctgagcgccgtgcagagctgggtcgcccgccactggctgcccgcaggttgtgaaacagcaattttcttcccaatgcgttcgaagaagatttttggaagcgtgcatcctgtgagaccaatgaagcttgaatcttttagtacttgcatttgggtcaaagccacagatgtattaaacaaaaccatcctgttttcttatggcacaaagtggaacccctatgagattcagctgtacctcagttcccagtccctagtgttggtggtgggtggaaaggagaacaagctggctgcagacactgtggtgtccctggggaggtggtcccacctgtgtggcacctggagttcagagcaggggagcatgtccctgtgggcaaacggggagctggtggctaccactgtagagatggccaaaagtcactctgttcctgagggtggactcctacagattggccaagaaaagaatggttgctgtgtaggtgggggctttgacgaatcattagcattttctggaagaatcacaggcttcaatatctgggatcgggttctcagcgaggaggagatacgggccagtggaggagtcgaatcctgtcacatccggggaaatgtcgtcgggtggggagtcacagagattcaggcgcacggaggagcccagtatgtttcttaagtgttgtgaaaatctacttgaagccaaaggagactcacattttaaatatgccagttggaaaagtctgaaaacttcggtgcgtaatagacgaatgaaggagagacttgagattgtctttgtttatcttggcaaaatactgaatacacagttgaagggaaggcttgagagagggctccgggatgttgttactaagccttatactgtggtgctttcagattaatgtctgcctctgtcagataaaccctcagataactaaacaggactggactctgaacagagggacgattgtgtgacttttttttttttttattttggttaattttattttggccagagacatttttatattggaagaataacaaaacaagctctgttgcccattgttcattctttctggtgtgtattttgtgacaaaagagatgatgagaaaaccatatttataccacaaagtgacttattaacgaacataaatgtagcttacgtgttataatccaatgttccatttgggagaaggtagttgtgtaatttatattgtgaaatgtatttgtattaattttatttttgtaaaagtctactgtaaataaaatgttttataaagctaaa

SEQ ID NO: 18 - protein, 381 aa, Mus musculus
MHLPAILLCALWSAVVAETSDDYELMYVNLDNEIDNGLHPTEDPTPCDCRQEHSEWDKLFIMLENSQMREGMLLQATDDVLRGELQRLRAELGRLAGGMARPCAAGGPADARLVRALEPLLQESRDASLRLARLEDAEARRPEATVPGLGAVLEELRRTRADLSAVQSWVARHWLPAGCETAIFFPMRSKKIFGSVHPVRPMKLESFSTCIWVKATDVLNKTILFSYGTKWNPYEIQLYLSSQSLVLVVGGKENKLAADTVVSLGRWSHLCGTWSSEQGSMSLWANGELVATTVEMAKSHSVPEGGLLQIGQEKNGCCVGGGFDESLAFSGRITGFNIWDRVLSEEEIRASGGVESCHIRGNVVGWGVTEIQAHGGAQYVS

SEQ ID NO: 19 - DNA, 1908 bp, Homo sapiens
  CDS (117)..(1262)
catttattaaggactctctgctccagcctctcactctcactctcctccgctcaaactcagctcacttgagagtctcctcccgccagctgtggaaagaactttgcgtctctccagcaatgcatctccttgcgattctgttttgtgctctctggtctgcagtgttggccgagaactcggatgattatgatctcatgtatgtgaatttggacaacgaaatagacaatggactccatcccactgaggaccccacgccgtgcgactgcggtcaggagcactcggaatgggacaagctcttcatcatgctggagaactcgcagatgagagagcgcatgctgctgcaagccacggacgacgtcctgcggggcgagctgcagaggctgcgggaggagctgggccggctcgcggaaagcctggcgaggccgtgcgcgccgggggctcccgcagaggccaggctgaccagtgctctggacgagctgctgcaggcgacccgcgacgcgggccgcaggctggcgcgtatggagggcgcggaggcgcagcgcccagaggaggcggggcgcgccctggccgcggtgctagaggagctgcggcagacgcgagccgacctgcacgcggtgcagggctgggctgcccggagctggctgccggcaggttgtgaaacagctattttattcccaatgcgttccaagaagatttttggaagcgtgcatccagtgagaccaatgaggcttgagtcttttagtgcctgcatttgggtcaaagccacagatgtattaaacaaaaccatcctgttttcctatggcacaaagaggaatccatatgaaatccagctgtatctcagctaccaatccatagtgtttgtggtgggtggagaggagaacaaactggttgctgaagccatggtttccctgggaaggtggacccacctgtgcggcacctggaattcagaggaagggctcacatccttgtgggtaaatggtgaactggcggctaccactgttgagatggccacaggtcacattgttcctgagggaggaatcctgcagattggccaagaaaagaatggctgctgtgtgggtggtggctttgatgaaacattagccttctctgggagactcacaggcttcaatatctgggatagtgttcttagcaatgaagagataagagagaccggaggagcagagtcttgtcacatccgggggaatattgttgggtggggagtcacagagatccagccacatggaggagctcagtatgtttcataaatgttgtgaaactccacttgaagccaaagaaagaaactcacacttaaaacacatgccagttgggaaggtctgaaaactcagtgcataataggaacacttgagactaatgaaagagagagttgagaccaatctttatttgtactggccaaatactgaataaacagttgaaggaaagacattggaaaaagcttttgaggataatgttactagactttatgccatggtgctttcagtttaatgctgtgtctctgtcagataaactctcaaataattaaaaaggactgtattgttgaacagagggacaattgttttacttttctttggttaattttgttttggccagagatgaattttacattggaagaataacaaaataagatttgttgtccattgttcattgttattggtatgtaccttattacaaaaaaaatgatgaaaacatatttatactacaaggtgacttaacaactataaatgtagtttatgtgttataatcgaatgtcacgtttttgagaagatagtcatataagttatattgcaaaagggatttgtattaatttaagactatttttgtaaagctctactgtaaataaaatattttataaaactaaaaaaaaaaaaaaaaaaaaaaaaaaaaaaaaaaaaa

SEQ ID NO: 20 - protein, 381 aa, Homo sapiens
MHLLAILFCALWSAVLAENSDDYDLMYVNLDNEIDNGLHPTEDPTPCDCGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 21 - DNA, 3016 bp, Mus musculus
  CDS (264)..(1682)
actgaggactttttcttcttcacagctgccttctgaaatgtgtggagcaactggtggatggtttcctagcctttcaaaggtagtttacaaatgatttgaagtgctgatcctttcagagtctcaacagattatttcttaaacagctcatacattgctgtttatgcatgaattagtagaaccgggccacgtagaaaacactgtgattgtacctgggagttggcgagaaaaccagtgctctcccttgattctgctgcacgaggagaatgggctgtgatcggaactgtgggctcattgctggagctgttattggtgcagtcctggctgtgtttggaggcattctcatgccagtcggagacatgcttattgggaagacaatcaaaagggaagttgtccttgaagaaggaaccactgctttcaaaaactgggttaaaacaggcaccactgtgtacagacagttttggatctttgatgtgcaaaacccagatgacgtggcaaagaacagcagcaaaatcaaggttaaacaaagaggtccttacacatacagagttcgttatctagccaaggaaaatataactcaggaccccgaggaccacactgtgtcttttgtacagcccaatggagccatctttgagccttcactgtctgttggaacagaggatgacaacttcacagttctgaatctggctgtagcagctgcaccacatatctaccaaaattcatttgttcaagttgtgctcaactctcttataaaaaagtccaagtcttctatgttccaaacaagatctttgaaagaactcttgtggggttacaaagatccattcctcagtttggttccatatcctataagtaccacagttggtgtgttttatccttacaatgacactgtagatggagtttataaagttttcaatggaaaggataacataagcaaagttgccataattgagtcctataaagggaaaaggaatttgtcctattggccaagctattgcgacatgattaatggcacagacgcagcctcctttccaccttttgttgaaaagtctcggacattgagattcttttcctctgacatttgcaggtctatctacgctgtgttcggatctgaaatcgaccttaaaggaatccccgtgtacagatttgttcttccagccaatgcctttgcatcacccctccagaatccagacaaccattgtttctgcactgaaaaagtaatctccaataactgtacatcttatggtgtgctagacattggcaaatgcaaagaaggaaagcctgtgtatatttcgcttccacatttcctacatgcaagtccagatgtttcagaacctattgaaggcttacatccaaatgaagatgagcataggacatacttagatgtggaacccataactggattcactctacaatttgcaaaacgactgcaggtcaacatattggtcaagccagctagaaaaatagaagcattaaagaatctgaagagaccttacattgtacctatactgtggctaaatgagactgggaccattggtgatgaaaaagcagaaatgttcaaaacacaagtgactgggaaaatcaagctccttggcatggtagagatggccttacttgggattggagtggtgatgtttgttgcttttatgatttcatattgtgcttgcaaatccaagaatggaaaataagtagtggatgagcctacatatacactggctacatctttggtaaagccgatctacaaaatgaaaacttaatatatgcttcgtttttacaaaacacacctatctttaggagaagaaatggtggtgtgtgctctctctctctttctctttctctcttattgcagatatatatttatttgtaaatatatatatatgcaataagtcacagcatatttcaaaagattaatatgtcactataggcaatattttttaataaaatcttgcacttttattaaaagtccatcatttgcaactgagtggacttcaatttctgtagacccaattatcctgtttggttctgatttactgatttgttccatgttggcaaatttcaagaatgtacattctgagaaatttttgttttccctcactggaggaaactgctatcatgactggggtggcccctttgtttatagcaaatttggcttgcaactgtcagcacatggcataagtataacatcttgaaagacttaagaatgaaaaatgaacaattcacatgtgagccactgcttatatattaagtctctccctctctggagttcttggctacagcaaggccagatatcacattggttttggttttgttgtttttgttttgtttttgttttttactctctgacacagagcttatgaaatggacttttttttttttttttagcataccttagctctttgtattttaagtatgtcgtcatgttccatgctgcatagctctttaaaaaacctgagtaggtttttctctttctgctcagctgcaactaataacaaccttggagagctgttatagtgttaaaagatgtaaatgataataaaagaattattaaatggaatcctacaaaagcaacaatgggctttaatatatatttgtggtaatatctcctgctttcataatcacccaaaaaaaggactggtttctaacattaaaagaagtcgttccttaaattcaacctttttgtctagttactattgaatcacagtatgttatttattgtattttattttgttttttgtagaagtgttctatctgagattctgtgtccttctgaatcatttaacctttctgctatgtcttaatgcaattgaacacagatattttgttataattttatagcagcagtgtgagactgaaggcaaatattttccagggtaagacacagtgataggttgttctacttcctttagcagcataatcaatttcacttagtattagtcccaataaattctggtataagacacaaaataaaaacaaacattttctgttctatacgtagaaaaataaaaggaaaggtgagacaact

SEQ ID NO: 22 - protein, 472 aa, Mus musculus
MGCDRNCGLIAGAVIGAVLAVFGGILMPVGDMLIGKTIKREVVLEEGTTAFKNWVKTGTTVYRQFWIFDVQNPDDVAKNSSKIKVKQRGPYTYRVRYLAKENITQDPEDHTVSFVQPNGAIFEPSLSVGTEDDNFTVLNLAVAAAPHIYQNSFVQVVLNSLIKKSKSSMFQTRSLKELLWGYKDPFLSLVPYPISTTVGVFYPYNDTVDGVYKVFNGKDNISKVAIIESYKGKRNLSYWPSYCDMINGTDAASFPPFVEKSRTLRFFSSDICRSIYAVFGSEIDLKGIPVYRFVLPANAFASPLQNPDNHCFCTEKVISNNCTSYGVLDIGKCKEGKPVYISLPHFLHASPDVSEPIEGLHPNEDEHRTYLDVEPITGFTLQFAKRLQVNILVKPARKIEALKNLKRPYIVPILWLNETGTIGDEKAEMFKTQVTGKIKLLGMVEMALLGIGVVMFVAFMISYCACKSKNGK

SEQ ID NO: 23 - DNA, 1870 bp, Homo sapiens
  CDS (211)..(1629)
gaaaaatccttcttagccattttaaagatagctttccaatgattagacgaattgattctttctgtgactcatcagttcctttcctgtaaaattcatgtcttgctgttgatttgtgaataagaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactaccacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagatttgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattgaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaaat

SEQ ID NO: 24 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 25 - DNA, 2419 bp, Mus musculus
  CDS (352)..(1644)
gcggtgaggcgggcggaccggccggccggtgactttgggagccactggggttcgagcgccgagcctgcagctcacgccccagctcgcggccgccgaggagagcgcgggaagcagccctgcgcacttgggaggcagaggcagaggcaagcaggtgtctgaggtcgaagccagcctggtctacatagctagttccaggactgctggggctgttagacagagaagctctgcctcgaatccctgtcccaaccgcaaccccaaagaagattggctgctgatactgctcaacttaatatcctaaagctgctatggcataaaaagtctttggggcaaaaaagactgcctgattgaagaatggcagaagcagtcttgattgatctctctggtttacaattgaatgcacagaaaaactgccatgagaccttactggagacactggatggtattcactaccaccacgctcccaaggccaagttcctttgcataatatgttgcagaaatgccagcaaggaaaaggatggggagtatggcctctgtgaattggaagcaggaaatggcttttcacgtctcgcgggaaaattcgagactgttagccatccctgcctggctgcctctttgtatacaattaaacaaaaaattgatgaagaaaatctgagctgcattaaggtgattgtccccgagcacaggaaactgttaatgaaggcttacgttggtcaactcttcaccgaggtgtatgagtttgagtttgaagatctacagggagcttggagggatagccttttgaaaccatccactggaataaacgtgaccacaactcaagaactagaagacatccagcatgaaatagaaacatacttgagaagcctgccagcgctgaaaggggacctaactattgtcacatctcctctgatcccagacaacttcctgcatggatttaccacacggacagggggcatatcttccgtacccactctcagctccctcaatcttttcagtagttccaaaaggagagatcccaaagtggttgttcaagaaaatgttcgcaggttggcaaatgctgccggatttaacgcagagaaattttaccgaataaagactgatcacgccagtgaagtgtgggttatggggaagaaggagcctgaatcttacgatgggatcgtcacaaatcagagaggagtcacaatcactgctcttggcgccgactgtatacccatcgtcttcgcagatcctgtgaaaaaggcatgtggggttgctcactccggctggaaaggtacattgttgggtgtggctatggctactgtgaatgctatgatagcagaatatggctgtgatgtggaagacattattgttgttctgggaccgtcagtgggttcttgctgcttcactcttcccaaagaatcagcagtctcctttcacagtcttcatccttcatgtgtgagacactttgactcaccgagaccctacgttgacatccgaaaagccaccaggattcttctagaaagaggaggaattcttccacagaacattcaagaccagaaggaagatcttgacctctgcacgtcctgccatcctgagaagtttttctcccatgtccgcgatggccttaacttcggtacacagattggcttcatatcccttagagaatgaggttcttggctggagttttgcataaaggatgtttcctgcatccttccacactggctgcaaaagaaaatacttagctttctgatttatataaaaccatgaggattacaaaagataattcatctttagggtatgctcttactgttacccaaagcccagtgatcttggtctaattcttatgacaactgatcaaaaaacccaatgtgatgtcattcctgaaggtcgttctctgtatttgtctataaatcaaggccagggctgttcagttaagtatttggtagatgccaacttttgtttaggcatggagcccagcacagggaattgaaatgaagtcagctttctcatttgaagcagatgcagctgggagatacctgttcccttaggtttccttatggcattctcaagcgccatgtttttgctgatccttccctccccttccctgtcccctgtgtccccttccactcccggtagcccctctttcctgtctcatgggctttaatacttctcccttgctcagtcctctctttatgttccctttctagtatcaacacctatatccacacctatgaccacaaatttacagatccatatatatgtatgtttacataaacagtaaaagttagaatttatatttatatataagaaaaaatacatgcagtgtttgttattcccagtctggattattttggtcagcataaaataattccagatctctcaattttcctacaagtttgataacttcattatggctaaataaaatattgtgtgtatgaactgc

SEQ ID NO: 26 - protein, 430 aa, Mus musculus
MAEAVLIDLSGLQLNAQKNCHETLLETLDGIHYHHAPKAKFLCIICCRNASKEKDGEYGLCELEAGNGFSRLAGKFETVSHPCLAASLYTIKQKIDEENLSCIKVIVPEHRKLLMKAYVGQLFTEVYEFEFEDLQGAWRDSLLKPSTGINVTTTQELEDIQHEIETYLRSLPALKGDLTIVTSPLIPDNFLHGFTTRTGGISSVPTLSSLNLFSSSKRRDPKVVVQENVRRLANAAGFNAEKFYRIKTDHASEVWVMGKKEPESYDGIVTNQRGVTITALGADCIPIVFADPVKKACGVAHSGWKGTLLGVAMATVNAMIAEYGCDVEDIIVVLGPSVGSCCFTLPKESAVSFHSLHPSCVRHFDSPRPYVDIRKATRILLERGGILPQNIQDQKEDLDLCTSCHPEKFFSHVRDGLNFGTQIGFISLRE

SEQ ID NO: 27 - DNA, 2135 bp, Mus musculus
  CDS (97)..(1782)
aagaggtaatccagactctgctcacgaaggaactcagcactgcatcctgccagctccaccgccaccactactgccacctccgctgccacctctgcgatgctcttccgagctgtgctgctctgcgctgccctggggctcagccaggcagcaaatccttgctgttccaatccatgtcaaaaccgtggggaatgtatgagcacaggatttgaccagtataagtgtgactgtacccggactggattctatggtgaaaactgtactacacctgaatttctgacaagaatcaaattactgctgaagcccaccccaaacacagtgcactacatcctgacccacttcaagggagtctggaacattgtgaacaacatccccctcctgcgaagtttaatcatgaaatatgtgctgacatccagatcatatttgattgacagtccacctacttacaatgtgcactatggttacaaaagctgggaagccttctccaacctctcctactacaccagggcccttcctcccgtagcagatgactgcccaactcccatgggtgtgaagggaaataaggagcttcctgattcaaaagaagtgctggaaaaggttcttctacggagagagttcatccctgacccccaaggctcaaatatgatgtttgcattctttgcccagcacttcacccatcagtttttcaagacagatcataagcgaggacctgggttcacccgaggactgggccatggagtggacttaaatcacatttatggtgaaactctggacagacaacataaactgcgccttttcaaggatggaaaattgaaatatcaggtcattggtggagaggtgtatccccccacagtcaaagacactcaggtagagatgatctaccctcctcacatccctgagaacctgcagtttgctgtggggcaggaagtctttggtctggtgcctggtctgatgatgtatgccaccatctggcttcgggagcacaacagagtgtgcgacatactcaagtttgacccagagctccttttcaaccagcagttccagtatcagaaccgcattgcctctgaattcaacacactctatcactggcaccccctgctgcccgacaccttcaacattgaagaccaggagtacagcttcaaacagtttctctacaacaactccatcctcctggaacatggactcactcagtttgttgagtcattcaccagacagattgctggccgggttgctgggggaagaaatgtgccaattgctgtacaagcagtggcaaaggcctccattgaccagagcagagagatgaaataccagtctctcaatgagtaccgcaaacgcttctccctgaagccgtacacatcatttgaagaacttacaggagagaaggaaatggctgcagaattgaaagccctctacagtgacatcgatgtcatggaactgtaccctgccctgctggtggaaaaacctcgtccagatgctatctttggggagaccatggtagagcttggagcaccattctccttgaaaggacttatgggaaatcccatctgttctcctcaatactggaagccgagcacctttggaggcgaagtgggttttaagatcatcaatactgcctcaattcagtctctcatctgcaataatgtgaaggggtgtcccttcacttctttcaatgtgcaagatccacagcctaccaaaacagccaccatcaatgcaagtgcctcccactccagactagatgacattaaccctacagtactaatcaaaaggcgttcaactgagctgtaaaagtctactgaccatatttatttatttatgtgaagaatttaatttaattatttaatatttatactgaattttttttcatgtaacatcttccataacagaaggcaatgttcttgaacaatgttacatttgtgaagattccctccggtgtttgtcctttaaatatgtgttacctgaaactgaaaggaaatcagcattcattcctctacataagccagtgagaagggaaatgaattttgatatctttatacttgaatttcagatcatgattagcttaacaagaaccaaggaaaaatgtatgaatatgtgagtgttgttacaagatgaaaaatgctgcaggtatcaaaaaaaaaaaa

SEQ ID NO: 28 - protein, 561 aa, Mus musculus
MLFRAVLLCAALGLSQAANPCCSNPCQNRGECMSTGFDQYKCDCTRTGFYGENCTTPEFLTRIKLLLKPTPNTVHYILTHFKGVWNIVNNIPLLRSLIMKYVLTSRSYLIDSPPTYNVHYGYKSWEAFSNLSYYTRALPPVADDCPTPMGVKGNKELPDSKEVLEKVLLRREFIPDPQGSNMMFAFFAQHFTHQFFKTDHKRGPGFTRGLGHGVDLNHIYGETLDRQHKLRLFKDGKLKYQVIGGEVYPPTVKDTQVEMIYPPHIPENLQFAVGQEVFGLVPGLMMYATIWLREHNRVCDILKFDPELLFNQQFQYQNRIASEFNTLYHWHPLLPDTFNIEDQEYSFKQFLYNNSILLEHGLTQFVESFTRQIAGRVAGGRNVPIAVQAVAKASIDQSREMKYQSLNEYRKRFSLKPYTSFEELTGEKEMAAELKALYSDIDVMELYPALLVEKPRPDAIFGETMVELGAPFSLKGLMGNPICSPQYWKPSTFGGEVGFKIINTASIQSLICNNVKGCPFTSFNVQDPQPTKTATINASASHSRLDDINPTVLIKRRSTEL

SEQ ID NO: 29 - DNA, 3394 bp, Homo sapiens
  CDS (121)..(1935)
cttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactgtcacaagatggcaaaatgctgaaagtttttacactgtcgatgtttccaatgcatcttccatgatgcattagaagtaactaatgtttgaaattttaaagtacttttggtcatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagtagaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatcttaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagtcttaagatcagagttcactttctttggactctgcctatattttcttacctgaacttttgcaagttttcaggtaaacctcagctcaggactgctatttagctcctcttaagaagattaaaaaaaaaaaaaaaa

SEQ ID NO: 30 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 31 - DNA, 1087 bp, Mus musculus
  CDS (92)..(268)
ggagtctggtgctgactggtgaattcagaagcaggaaagagggacagagtggattttacaaccagaataaagctgggaaatgcccccgtgaatggagccaaagaaaggttggagtttggggtatggtgtgtatttgaagattcaaactccacgtggtcttcaggagctcaaacactctcacacagaacagaaaggaagtctccaaaacgaatgtgaaattgacaaatcaccgcactgcttcaggaccccagcccatggaatgctgtgatcaacattcagagtcgatcatcctacatcaattaccctaatctaggaaatcccacagagccaggaccaaagaacgtaaaacaaaaacaaacaaaacccagcctgtgaacattagcaatagcacagtacctgagactacacagaagcctgggaaagtcacctgtgaatgctgatgctctacagcatcaccatggtagagaaactacaaaagaagcttttgattctgaagctgagcctgatcctccatgatcacaacattgaagtctaatcctctacatcaaatattttaactgtgagatttgtagacccaacagattgctactcacctgttgccatgactgaagacttgaaaacattccaaaacacgttcctgattgcatagccctaaaattccttcctagaagttcattccgttcagtgggttctataatttctcttctaaattatactctgtcacgtcaaacatcatgacaattttcaaagtttactacttccagaaactttatttctaacaaatacttactgagctaatgtatgaggtgttataattgtatgacaaatttaatacagcacatatttataatgacatcgaaacacatatatttttaccacaaagcatatttgaagaaaactttataaaataatattaacctatagttcgtgcagttctcacctggatattatatgctactgtatgttagttttcaaagtgatgatcttaacctataatgttaatttgaccctttgatagaaaatttttttctggtaattacttgaattgtttatttggttttaaaattaacaataaagaatcaaatat

SEQ ID NO: 32 - protein, 58 aa, Mus musculus
MEPKKGWSLGYGVYLKIQTPRGLQELKHSHTEQKGSLQNECEIDKSPHCFRTPAHGML

SEQ ID NO: 33 - DNA, 1751 bp, Mus musculus
  CDS (122)..(1069)
gtccgctcccgcctcgccgcgcgcacagcgctcagtccgtccgccgccgcgcagcgcgactgtcctccgagccgtcccgcgccgcacctccgcgagtcgccctcgccgctccgcgcgcagcatggcgggtccccaggccctcgcgttcgggctcctgctcgcggtggtcacagcgacgctggccgcggctcagagagactgtgtctgtgacaactacaagctggcaacaagttgctctctgaatgaatatggtgaatgccagtgtacttcctatggtacacagaatactgtcatttgctccaaactggcgtctaaatgcttggcgatgaaagcagaaatgactcacagcaagtctgggaggaggataaagcccgaaggggcgatccagaacaacgatgggctgtacgaccccgactgcgacgagcaggggctcttcaaagccaagcagtgcaacggcaccgccacgtgctggtgtgtcaacaccgccggagtccgaagaaccgacaaggacacggagatcacgtgctccgagcgcgtgaggacctactggatcatcattgaactaaaacacaaagaaagagaaagcccctacgaccatcagagcttgcagactgcgcttcaagaggcgttcacatctcgatataagctgaatcagaaatttatcaaaaacattatgtatgagaataatgttatcaccattgatctgatgcaaaactcttctcagaaaacacaagacgacgtggacatagctgatgtggcttactattttgaaaaagatgtgaagggggagtccctgttccattcttctaagagcatggacctgagagtgaacggagagccgctcgatctggaccccgggcagactctgatttactacgttgatgaaaaggcacccgagttctccatgcagggcctcacggccgggatcatcgctgtcattgtggtggtgtcattagcagtcatcgcggggattgttgtcctggttatatctacaaggaagaaatcagcaaaatatgagaaggctgagataaaggagatgggtgagatccacagagagcttaatgcctagccgtgctgagtgctgaactgaggaggggccgcccgaccggaagtggcagaagagctcggactgcagatgtataaacctggggaagatgaagacctgcgaagggttactgctttgatagttactttgttagtttcacatttgtaacagtgaaatttgtactcgtaaatacaagcagctggacaccggcattaccgatcgtaaaattagacgaacgtcttataggtgcaggtccagtgtggtactcagaacttagcctgcaaagttaagagagttgatgcttattatgacagagtgtgcgtcgcaaacattccaacagtagaatgcggtgactagtctcattttttttttttttttgtgattaaggctgcccttctatatacctgagtcttgtacataataaacttttttttaatgaaataaaacattttaaagtgagtttctaagtttgtttgaatcaaattttcctagcatgtgcataattaagataatagatgtctaaatgctctggcactgctaactggtacaaacctgtaattctgtacttgggaggtagaggtaggagggttagcgcttccgaggtagctgctgtgtatctgctctgccactgactggccttgactatccaacaccctatctgaaagaaataaaaatcaaacttaaaaaaaaaaaaaaaaaaaaaaaa

SEQ ID NO: 34 - protein, 315 aa, Mus musculus
MAGPQALAFGLLLAVVTATLAAAQRDCVCDNYKLATSCSLNEYGECQCTSYGTQNTVICSKLASKCLAMKAEMTHSKSGRRIKPEGAIQNNDGLYDPDCDEQGLFKAKQCNGTATCWCVNTAGVRRTDKDTEITCSERVRTYWIIIELKHKERESPYDHQSLQTALQEAFTSRYKLNQKFIKNIMYENNVITIDLMQNSSQKTQDDVDIADVAYYFEKDVKGESLFHSSKSMDLRVNGEPLDLDPGQTLIYYVDEKAPEFSMQGLTAGIIAVIVVVSLAVIAGIVVLVISTRKKSAKYEKAEIKEMGEIHRELNA

SEQ ID NO: 35 - DNA, 1458 bp, Homo sapiens
  CDS (183)..(1127)
ccttcggcgagcgagcaccttcgacgcggtccggggaccccctcgtcgctgtcctcccgacgcggacccgcgtgccccaggcctcgcgctgcccggccggctcctcgtgtcccactcccggcgcacgccctcccgcgagtcccgggcccctcccgcgcccctcttctcggcgcgcgcgcagcatggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataactatataatttgaagattatagaagaagggaaatagcaaatggacacaaattacaaatgtgtgtgcgtgggacgaagacatctttgaaggtcatgagtttgttagtttaacatcatatatttgtaatagtgaaacctgtactcaaaatataagcagcttgaaactggctttaccaatcttgaaatttgaccacaagtgtcttatatatgcagatctaatgtaaaatccagaacttggactccatcgttaaaattatttatgtgtaacattcaaatgtgtgcattaaatatgcttccacagtaaaatctgaaaaactgaaaaaaaaaaaaaa

SEQ ID NO: 36 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 37 - DNA, 1400 bp, Mus musculus
  CDS (103)..(1053)
ccacgcgtccgcccacgcgtccgcccacgcgtccgcccacgcgtccgcctaatttaggcaaaggctcccgtctgttccctttgaaccgtttctgcagcaaccatggccgccttcgtgacgctccttaccggagccaagatgcccattgtgggcctgggcacctggaagtctcccccagctaaagtcagggaagctgtgaaggtggccattgatgctgggtatcgccacattgattgtgcctatgtctatcagaatgagagcgaggtgggagaagccatccaagagaagatccaggagaaggctgtgaagcgggaggacctcttcatcgtcagcaagctgtggtccaccttctttgagaaaagcctggtgaagaaagccttccagaacaccctctcggatctgaagcttgactacctggacctgtatctaatccactggccacagggatttcagtctggaaatgtctttttacccacagatgataaaggcagtattctcagcagtaaatatacgttcttggatgcttgggaggccatggaggaactggtggaccagggactggtgaaagctctgggtgtctccaacttcaaccacttccagattgaaaggctcctgaacaagcctggactgaaacataagccagtgaccaaccaggttgagtgtcacccttacctcacccaggaaaaactgacccagtactgtcactccaagggcatcaccatcacagcctacagtcccctgggctccccagacaggcctagtgccaaaccagaggaccccctattactggagattcccaagatcaaagagattgctgcaaagcacaagaagactgcagcacaggttctgattcggttccatatagagaggaacgtggtggtgatccccaagtctgtgacaccttctcgaatacaggagaacattcaggtcttcgacttccagttaagtgaggaggacatggccgccattctcagcttcaacaggaactggagggcctgtggcctgtttgcagcaagtcataacgaggacttccctttccatgcggaatactgaagccgagtaccagatgcgcttgcctggaggacactcttcactgatatgcaacccgctcctactgactcctatttttgccaagcctattggatatcataccaaaccagcctcaacagtgcaattctgcatttaaattctcatgttcaactagaagcagactgtcacaggaagatgcatctcagcaaagggcaatgtttgtcactaatttgacctggccggatgtttgtcagaaaccaggaacactaataacactgaagattaaaaagataaaataataaaaatagcaataatcgtaaaaaaaaaaaaaaaaaaaaaaaaaaaaaaaaaaaaaaaaaaaaaaaaaaaaa

SEQ ID NO: 38 - protein, 316 aa, Mus musculus
MAAFVTLLTGAKMPIVGLGTWKSPPAKVREAVKVAIDAGYRHIDCAYVYQNESEVGEAIQEKIQEKAVKREDLFIVSKLWSTFFEKSLVKKAFQNTLSDLKLDYLDLYLIHWPQGFQSGNVFLPTDDKGSILSSKYTFLDAWEAMEELVDQGLVKALGVSNFNHFQIERLLNKPGLKHKPVTNQVECHPYLTQEKLTQYCHSKGITITAYSPLGSPDRPSAKPEDPLLLEIPKIKEIAAKHKKTAAQVLIRFHIERNVVVIPKSVTPSRIQENIQVFDFQLSEEDMAAILSFNRNWRACGLFAASHNEDFPFHAEY

SEQ ID NO: 39 - DNA, 1655 bp, Mus musculus
  CDS (360)..(1469)
cggagaccgcagctatggcgtcgttactttggggaggcgacgccggggcggcggagagcgagcggctgaacagccacttttcaaatctcgtccatcccaggaagaaccttcgtggtattaggagtaccacagtcccaaacatagacggctctcttaacacggaagatgatgatgatgatgaagacgatgtagtggatttggcagccaattcactcttaaacaagctaatccgtcagtccttaatagaatccagccaccgagtggaagtcttacagaaggatcccagctctccactctactcggtgaaaacctttgaagagctacggctaaaagaagaattactaaaagggatttatgcaatgggatttaaccgaccatccaaaatccaagagatggccctccccatgatgctagcacaccctccccagaacctcatagcacagagccagtccggcacaggaaaaactgctgcctttgtcttagcaatgctaagccgagtgaatgccttggaactgtttccacagtgcctctgcctggctcctacctatgaactggctctgcagaccggtcgtgtggttgaaaggatgggcaagttctgcgtggatgttgaagtgatgtatgccattcgaggaaaccgaattccaaggggcaccgaagtcaccaaacagattattattggtactcctgggactgtcctagattggtgtttcaagcgaaaactgattgatttgacaaagatccgtgtgtttgtcctggatgaagcagatgtgatgattgacacccaaggattctccgatcagagcatccgtatccaaagagctttaccctctgaatgccagatgctcctcttttcggcaacctttgaggactctgtgtggcagtttgctgaacgaatcattcctgaccccaatgttatcaagttgcgaaaagaggaactgaccctgaacaacatccggcagtattacgtgttgtgtgagaacaggaaaggcaaataccaagccctctgtaacatctacggtggcatcaccattggccaagccattatcttctgccagacacgtcgaaatgccaagtggctgactgtggagatgatgcaggatggccaccaggtgtccctgctaagtggggaactcacagtggagcagcgagcttccatcattcagagattccgagatggcaaagagaaggttctcataacgaccaatgtctgtgctcgagggattgatgtgaagcaggtcaccatcgtcgtgaactttgacctccctgtaaaccaatcagaagagccagactatgagacctacctccaccgtatagggcggacgggacgctttgggaaaaaaggtctcgccttcaacatgattgaagtggataagctgcccctgcttatgaaaatacaagaccccttcaatagcaatattaagcagcttgacccagaagacatggatgagattgagaagatcgaatattgaagagagagagagaagagaggccttcgttgtttgtgggctagtttacgtgagaatgtttcagcagagtttgaagataattttctatgttgagactctgtcaaggtgaaactgtcacagataaaaaataaatgccatgactctcttcaaaaaaaaaaaaaaaaaaaaaaaaaaaaaaaaaaaaaaaaa

SEQ ID NO: 40 - protein, 369 aa, Mus musculus
MGFNRPSKIQEMALPMMLAHPPQNLIAQSQSGTGKTAAFVLAMLSRVNALELFPQCLCLAPTYELALQTGRVVERMGKFCVDVEVMYAIRGNRIPRGTEVTKQIIIGTPGTVLDWCFKRKLIDLTKIRVFVLDEADVMIDTQGFSDQSIRIQRALPSECQMLLFSATFEDSVWQFAERIIPDPNVIKLRKEELTLNNIRQYYVLCENRKGKYQALCNIYGGITIGQAIIFCQTRRNAKWLTVEMMQDGHQVSLLSGELTVEQRASIIQRFRDGKEKVLITTNVCARGIDVKQVTIVVNFDLPVNQSEEPDYETYLHRIGRTGRFGKKGLAFNMIEVDKLPLLMKIQDPFNSNIKQLDPEDMDEIEKIEY

SEQ ID NO: 41 - DNA, 1799 bp, Homo sapiens
  CDS (365)..(1474)
ggcgggagcagcaatcgcagccatggcgtcgttactgtggggaggcgacgcaggggcggcggagagcgagcggctgaacagccacttttcaaacctcagccaaccccggaagaacctttggggtattaagagtactgcagtccgaaacatagatggttctattaataacatcaatgaagatgatgaagaagatgtagtggatttggcagctaattcactcttaaacaagttaatccatcaatccttagtagaatccagtcaccgtgtggaagttttacagaaggatcccagctctccactttactcagtaaagacatttgaagagctgcggctaaaggaagagttactaaaaggaatctatgcaatgggatttaataggccatctaaaatccaagagatggctctccctatgatgctggcacatccaccccagaacctcatagcacagagccagtctggaacaggaaagacagcggcatttgtgttggcaatgttaagcagagttaatgccttggaattgttcccacagtgcctctgcctagctcctacttatgaattggctctgcaaactggccgtgtggttgagcagatgggaaaattctgtgtggatgttcaagtgatgtatgccattcgagggaatcgaattcccagaggcaccgacatcactaaacagattataattggcactcctgggactgtcctagattggtgttttaaactaaaattgattgatttgactaagattcgtgtgtttgtcctggatgaagcagatgtgatgattgacactcaaggattctcagatcatagtattcgtattcaaagagctctaccctccgaatgccaaatgctcctcttttcagcaacctttgaggactctgtgtggcactttgctgagcgaatcatccctgaccctaatgttatcaagttacgcaaagaggagctcacactgaacaacatccggcaatattacgtgctgtgtgagcacaggaaagacaaataccaagctctgtgcaacatttatggcagcatcaccattggtcaggccatcatcttctgccagactcgtcgaaacgctaagtggttgaccgtggagatgatacaggatggccaccaggtgtctttgttaagcggggagctgaccgtggagcagcgagcttccatcattcagaggtttcgggatgggaaagagaaggttctcataacaactaatgtttgcgcccgagggattgatgtgaagcaggtcacaattgttgtgaactttgatctccctgtaaaacaaggagaggagccggactatgagacctacctccaccgcatagggcggacggggcgctttgggaaaaaaggccttgccttcaacatgattgaagtagatgagctgccctcgctcatgaaaatccaggaccactttaacagcagtattaagcaactcaacgctgaagacatggatgaaattgaaaagattgactattgaagaaagaactttattgtttgtgcagtatcctagtttatgtgagaatgtctcagtgggttttgaaggtaatttttttatgttgaggctttttcgacgtgaaactgtcacagatggcaaaataaatgtcacggtacttagttttaagacatgaggtctttttgaagccaaattgatgtgaagcttgtgatccttttgaataaaaaaaaggcaagattaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - protein, 369 aa, Homo sapiens
MGFNRPSKIQEMALPMMLAHPPQNLIAQSQSGTGKTAAFVLAMLSRVNALELFPQCLCLAPTYELALQTGRVVEQMGKFCVDVQVMYAIRGNRIPRGTDITKQIIIGTPGTVLDWCFKLKLIDLTKIRVFVLDEADVMIDTQGFSDHSIRIQRALPSECQMLLFSATFEDSVWHFAERIIPDPNVIKLRKEELTLNNIRQYYVLCEHRKDKYQALCNIYGSITIGQAIIFCQTRRNAKWLTVEMIQDGHQVSLLSGELTVEQRASIIQRFRDGKEKVLITTNVCARGIDVKQVTIVVNFDLPVKQGEEPDYETYLHRIGRTGRFGKKGLAFNMIEVDELPSLMKIQDHFNSSIKQLNAEDMDEIEKIDY

SEQ ID NO: 43 - DNA, 2999 bp, Mus musculus
  CDS (249)..(1397)
aatctttagggtttgagcaggagaaataccaacagatcttttctccctccagtgacactcgtcccgcttcaacagaggtgaacagacgcattctttaaaagagtattcagttcaagggaatgaagaactgagaattatcttggtgaatggattcaaatatatggaataagagtatgctgaagatttgatccgttttgaagaactataactgtccatttatctaatcggtaacaacaaaacataaaaaaatgaactcaactctgttctccaaggttgaaaatcactcaattcactataatgcctcagagaattctccacttctggcttttgaaaatgatgactgccacctgcccttggctgtgatattcaccttggctctcgcttatggggcggtgattattcttggcgtctctggaaacctggcattgatcataatcattctgaaacagaaggagatgagaaatgtcaccaacattctgatcgtgaacctctccttctcagacttgctcgttgcggtcatgtgtctcccgttcacttttgtatatacactgatggaccactgggtcttcggggagaccatgtgcaaactgaatccctttgtacagtgtgtctccatcacagtatccattttctcgctggttctcatcgctgtggaacggcatcagctaatcatcaacccaagagggtggagaccaaacaatagacatgcttacataggcattactgtcatttgggtccttgcagtggcttcttctctgccctttgtgatctatcaaattctgaccgacgagcccttccaaaatgtgtcacttgcggcgttcaaggacaagtatgtgtgctttgacaaattcccatctgactctcacaggctgtcttacacgactctcctcctggtgctgcagtatttcggcccactctgctttatattcatatgctacttcaagatatacattcgcttgaaaaggagaaacaacatgatggacaagatccgggacagtaagtacaggtccagtgagaccaagcgaatcaacatcatgctgctctccattgtggtcgccttcgccgtctgctggctgccccttaccatcttcaacactgtgttcgactggaaccaccagatcattgccacctgcaaccacaatctgctgtttctgctctgtcacctcaccgccatgatctccacctgcgtcaaccccatcttttatggattcctgaacaaaaatttccagagagacttgcagttcttcttcaacttttgtgacttccggtctcgagacgatgactacgagaccatagccatgtctaccatgcatacggatgtgtccaagacgtctctgaagcaggctagcccagtcgcatttaaaaaaatcagtatgaatgacaatgaaaaagtctgaagctgctcagagcatatggtcccaggccatatctgtggaaaaacaagcacagcctgccgcatgctttctttacctatgctctgggggaacggaatgaggcgcgcttggaaagcccaggacatctgtgttgggtttgactgcttttgatggttgccctgattacttagaaatctagattactttgtaatctatctctggcaacagttttgactagatgtcctgtgtgtgtgtgtgtgtgtgtgtgtgtgtgtgtgtgtgtgtgtgtgtgtatgtgtgtgcgcgcgcgcgcgcgcgcacgtgcaaaagagaaagagagagagagaaggagagagacagacagactgcctgtctgtctgtctgtctgtctttctttatgtatgtgtttgaattatgcatatgacaaagagttttacattgtgtttgttggagtgaatttctctgaagtaatgtcatgagctcatttcaaaagcagtcaccacctgatattctcaagatactgaattttcaagatcagatgagatttccaagaccccggactaccttcgttccctgctaggcatcatcttagtctgtcacaagggtgacagtatacaaaagtcaccttttaaaatgtgcctgagtcaaaagagtgtctgaagtcatttggcagcatcttttccttttcctctctatttctgtaaggactcaatttcttatactcctaattgagtcttgcctaagataaagataagtaaagaacaccttttcagctgtggatatcaccaggagttaggccacccacaagaatgaagtggcagctgggcgtggtggcacacacctttaatcccagcacttgggaggcagaggcaggtggatttctgagtttgaggccagcctggtctacaaagtgagttccagaacagccagggctatacagagaaaccctgtctcgaaagacccccccccccaaaaaaaaagaatgaagtggcaaaacaggtccctgattttaaaatttttctgacaaccaaagcattttagggtaattagtttaaaatgtaaactagtcttgctgcagataggtaaattgcatccaaaggacttgataatcgaaaggttttgcattgttcatcagagtgctgagctctatcaagctttctggcacaagcatgcgactcagagcatttctaactcatactgcacagacgcgtaagatgcgttttctacacagccgtgcctgcatagtaactagtgtttaactctcaccatttatccttcactgcagaggacacagggacaatgtcaatgtatctacccttcagctaacagatgtctgtacctaaaactttcacgcagcctaacttgtataaactgtatacaagttcaagttcatcctaacttgtataaactgtataatgtgtggagttttataatcatatactattatatcatagagtactgagtagccctgccatattgatatatttactttcatgtatcttgtaatcatgatttagactcagaaaagatactttgaagaacaagacagtttcaatgtattgtacaaaatgttgcctgtgtgtgattttagaagggcagacacttctgaattaaaactgagctttt

SEQ ID NO: 44 - protein, 382 aa, Mus musculus
MNSTLFSKVENHSIHYNASENSPLLAFENDDCHLPLAVIFTLALAYGAVIILGVSGNLALIIIILKQKEMRNVTNILIVNLSFSDLLVAVMCLPFTFVYTLMDHWVFGETMCKLNPFVQCVSITVSIFSLVLIAVERHQLIINPRGWRPNNRHAYIGITVIWVLAVASSLPFVIYQILTDEPFQNVSLAAFKDKYVCFDKFPSDSHRLSYTTLLLVLQYFGPLCFIFICYFKIYIRLKRRNNMMDKIRDSKYRSSETKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPVAFKKISMNDNEKV

SEQ ID NO: 45 - DNA, 2775 bp, Homo sapiens
  CDS (267)..(1421)
aatctttaggatctgagcaggagaaataccagcggatcttccccactctgctcccttccattcccacccttccttctttaataagcaggagcgaaaaagacaaattccaaagaggattgttcagttcaagggaatgaagaattcagaataattttggtaaatggattccaatatggggaataagaataagctgaacagttgacctgctttgaagaaacatactgtccatttgtctaaaataatctataacaaccaaaccaatcaaaatgaattcaacattattttcccaggttgaaaatcattcagtccactctaatttctcagagaagaatgcccagcttctggcttttgaaaatgatgattgtcatctgcccttggccatgatatttaccttagctcttgcttatggagctgtgatcattcttggtgtctctggaaacctggccttgatcataatcatcttgaaacaaaaggagatgagaaatgttaccaacatcctgattgtgaacctttccttctcagacttgcttgttgccatcatgtgtctcccctttacatttgtctacacattaatggaccactgggtctttggtgaggcgatgtgtaagttgaatccttttgtgcaatgtgtttcaatcactgtgtccattttctctctggttctcattgctgtggaacgacatcagctgataatcaaccctcgagggtggagaccaaataatagacatgcttatgtaggtattgctgtgatttgggtccttgctgtggcttcttctttgcctttcctgatctaccaagtaatgactgatgagccgttccaaaatgtaacacttgatgcgtacaaagacaaatacgtgtgctttgatcaatttccatcggactctcataggttgtcttataccactctcctcttggtgctgcagtattttggtccactttgttttatatttatttgctacttcaagatatatatacgcctaaaaaggagaaacaacatgatggacaagatgagagacaataagtacaggtccagtgaaaccaaaagaatcaatatcatgctgctctccattgtggtagcatttgcagtctgctggctccctcttaccatctttaacactgtgtttgattggaatcatcagatcattgctacctgcaaccacaatctgttattcctgctctgccacctcacagcaatgatatccacttgtgtcaaccccatattttatgggttcctgaacaaaaacttccagagagacttgcagttcttcttcaacttttgtgatttccggtctcgggatgatgattatgaaacaatagccatgtccacgatgcacacagatgtttccaaaacttctttgaagcaagcaagcccagtcgcatttaaaaaaatcaacaacaatgatgataatgaaaaaatctgaaactacttatagcctatggtcccggatgacatctgtttaaaaacaagcacaacctgcaacatactttgattacctgttctcccaaggaatggggttgaaatcatttgaaaatgactaagattttcttgtcttgctttttactgcttttgttgtagttgtcataattacatttggaacaaaaggtgtgggctttggggtcttctggaaatagttttgaccagacatctttgaagtgctttttgtgaatttatgcatataatataaagacttttatactgtacttattggaatgaaatttctttaaagtattactattaactgacttcagaagtacctgccatccaatacggtcattagattgggtcatcttgattagattagattagattagattgtcaacagattgggccatccttactttatgataggcatcattttagtgtgttacaatagtaacagtatgcaaaagcagcattcaggagccgaaagatagtctgaagtcattcagaagtggtttgaggtttctgttttttggtggtttttgtttgttttttttttttttcaccttaagggaggatttaatttgctcccaactgattgtcacttaaatgaaaatttaaaaatgaataaaaagacatacttctcagctgcaaatattatggagaattggggcacccacaggaatgaagagagaaagcagctccctaacttcaaaaccattttggtacctgacaacaagagcattttagagtaattaatttaataaagtaaattagtattgctgcaaatagttaaattatatttatttgaattgatggtcaagagattttccattttttttacagactgttcagtgtttgtcaagctttctggcataaatatgtactcaaaaggcatttccgcttacaatttgtagaaacacaaaatgcgttttccatacagcagtgcctatatagtgactgatttttaactttcaatgtccatctttcaaaggaagtaacaccaaggtacaatgttaaaggaatattcactttacctagcagggaaaaatacacaaaaactgcagatacttcatatagcccattttaacttgtataaactgtgtgacttgtggcgtcttataaataatgcactgtaaagattactgaatagttgtgtcatgttaatgtgcctaatttcatgtatcttgtaatcatgattgagcctcagaatcatttggagaaactatattttaaagaacaagacatacttcaatgtattatacagataaagtattacatgtgtttgattttaaaagggcggacattttattaaaatcaatattgtttttgctttttcaaaaaaaaaaaaaaaa

SEQ ID NO: 46 - protein, 384 aa, Homo sapiens
MNSTLFSQVENHSVHSNFSEKNAQLLAFENDDCHLPLAMIFTLALAYGAVIILGVSGNLALIIIILKQKEMRNVTNILIVNLSFSDLLVAIMCLPFTFVYTLMDHWVFGEAMCKLNPFVQCVSITVSIFSLVLIAVERHQLIINPRGWRPNNRHAYVGIAVIWVLAVASSLPFLIYQVMTDEPFQNVTLDAYKDKYVCFDQFPSDSHRLSYTTLLLVLQYFGPLCFIFICYFKIYIRLKRRNNMMDKMRDNKYRSSETKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPVAFKKINNNDDNEKI

SEQ ID NO: 47 - DNA, 2025 bp, Mus musculus
  CDS (96)..(1274)
ctacacgcggcccgctgggctggcgtcccaaagccgtccctaacaccatcaagcagagagagcgcggcctctgaggggcgccggacgccggagccatgaccctccgccgactcaggaagctgcagcagaaggaggaggcgactgccgcgccggaccccgctggccgggctcccgactccgaagccgctcgcgccgccccgctaccctccggccctccggctgccgccgctccgcccggagcccccggggaggagctgtacgcagcactggaggactatcaccctgccgagctgtaccgcgcgctcgccgtgtccgggggtaccctgcctcgccgaaagggctcaggattccgttggaagaatttcactcagagtcctgaacagcaacggaaggttctaactttggagaaaggggacaaccagacctttggcttcgagatccagacatatggccttcaccaccgagaggagcagcgtgtagagatggtgaccttcgtctgccgagttcatgaatccagccctgcccagctggctgggctcacgccaggggacactattgccagcgtcaacggtctcaacgtggaaggcatccggcaccgggagattgtcgatatcatcaaggcgtctggcaacgttctcaggctggaaactctgtacgggacgtccattcggaaggcagaactggaggcgcgcctgcagtaccttaagcaaaccctgtacgagaagtggggagaatacaggtcacttatggtacaggagcagcggctagtacatggcctggtggtgaaagacccaagcatctatgacacactggagtccgtgcgctcctgcctctacggcgcaggcttgcttcccgggtcgctgccctttgggcctctgctcgccgcgcctgggagtgcccgcgggggtgcgcggcgggccaaaggggacacagacgacgccgtgtaccacacgtgcttctttgggggcgccgagccgcaggcactgccacccccgccaccccccgcgcgcgcgttaggcccgagctccgcggagacccccgcgtccgtgctgtttcccgcacctcgctccacgctcagccgcagcgccagtgtgcggtgcgcgggcccgggcggcggcgggggcgcgccgggcgcactctggactgaggcccgcgagcaggccctgtgtggcgccggcctacgcaagaccaagtaccgcagcttccgcagacggctgctcaagttcatccccggactcaaccgctccctggaggaagaggagagccagctgtaggggcgcgggtgggcaggtgggaagtatttatttatttattcatcgtaacagcctgtgcaaaaagtaggggcggtagtgtcggtccaaggaccccgggagcccaaagctgcgggagagggtccttgctagctctggtctttctggtcggttcattggcttgtctgctgaacaaaaaaaaaagggggggggggctagttgctttctgccctcccctaaaccacagcaggagctctacagggggagaatagacactgggaattgggagaaatctgggggaccaaagagggctgtaccaggctaaagtctgggtcagtcgtgccttgtgggggtcctgtcccaagaaaccctctcctcagatatactggctccccagcccttccttagccttggggagaggagatccacgatggatcattccccaccctacagagacagacagacagatacacacacacacacacacacacacacacacacacacacatcaatggaccagtgggccccaagggggcaaggtgctggtcccctccctccaaccttgacccctaagggcttaacccctgtccaggggcctataactaagtggggtcctgccaggccggggtcctgggttctgtgctccttggggaacaagaatgagttcattcaggtggggtggggcagcctggactgttccaccgttttctgcatatgttgcttctgaaccacaaactgtataaaattgatgggttttttttgcaaaaaaaaaaaa

SEQ ID NO: 48 - protein, 392 aa, Mus musculus
MTLRRLRKLQQKEEATAAPDPAGRAPDSEAARAAPLPSGPPAAAAPPGAPGEELYAALEDYHPAELYRALAVSGGTLPRRKGSGFRWKNFTQSPEQQRKVLTLEKGDNQTFGFEIQTYGLHHREEQRVEMVTFVCRVHESSPAQLAGLTPGDTIASVNGLNVEGIRHREIVDIIKASGNVLRLETLYGTSIRKAELEARLQYLKQTLYEKWGEYRSLMVQEQRLVHGLVVKDPSIYDTLESVRSCLYGAGLLPGSLPFGPLLAAPGSARGGARRAKGDTDDAVYHTCFFGGAEPQALPPPPPPARALGPSSAETPASVLFPAPRSTLSRSASVRCAGPGGGGGAPGALWTEAREQALCGAGLRKTKYRSFRRRLLKFIPGLNRSLEEEESQL

SEQ ID NO: 49 - DNA, 1950 bp, Homo sapiens
  CDS (57)..(1244)
ccagccccgccgaggggagccagcgccgtctctgaggggcgtccggcgccggagccatgaccctccgccgactcaggaagctgcagcagaaggaggaggcggcggccaccccggaccccgccgcccggactcccgactcggaagtcgcgcccgccgctccggtcccgaccccgggaccccctgccgcagccgccacccctgggcccccagcggacgagctgtacgcggcgctggaggactatcaccctgccgagctgtaccgcgcgctcgccgtgtccgggggcaccctgccccgccgaaagggctcaggattccgctggaagaatctcagccagagtcctgaacagcagcggaaagtgctgacgttggagaaggaggataaccagaccttcggctttgagatccagacttatggccttcaccaccgggaggagcagcgtgtggaaatggtgacctttgtctgccgagttcatgagtctagccctgcccagctggctgggctcacaccaggggacaccatcgccagcgtcaatggcctgaatgtggaaggcatccggcatcgagagattgtggacatcattaaggcgtcaggcaatgttctcagactggaaactctatatgggacatcaattcggaaggcagaactggaggctcgtctgcagtacctgaagcaaaccctgtatgagaagtggggagagtacaggtccctaatggtgcaggagcagcggctggtgcatggcctggtggtgaaggaccccagcatctacgacacgctggagtcggtgcgctcctgcctctacggcgcgggcctgctcccgggctcgctgcccttcgggcctctgctcgccgtgcccgggcgtccccgcggaggcgcccgacgggccaggggcgacgccgacgacgccgtctaccacacgtgcttcttcggggactccgagccgccggcgctgccgcccccgccgcccccggcccgcgccttcggcccgggccccgccgagacccctgccgtggggccgggccctgggccgcgggccgcgctgagccgcagcgccagtgtgcggtgcgcgggccctggcgggggcggaggcgggggcgcgccgggcgcgctctggactgaggctcgcgagcaggccctatgcggccccggcctgcgcaaaaccaagtaccgcagcttccgccggcggctgctcaagttcatccccggactcaaccgctccctggaggaggaggagagccagctgtaggggcgggggcgggcagggaggtatttatttatttattcgcaacagccagcgctaaaagagggggaggccgagccaagaggaccccaggagcccagagcagcgggagagggtccttcctagcctcggcccgccgggtcggttcctggctggtgtctgctgagggagtggggggcccagccccttctcttctcccccgccaaaccacagtgggagctggggcagggggagagccaggcaatcgggggccaaagatgggggtgctcgcctacagtctgcatctgtagtgccttgtggggtatccaggaacaccctcccagcaggggatgggaaccctgtcccatgaagccctctcctcagctttacttgctcccccgcccttagccttggggagaaatggcccgtggtgggctgaccccccaccctccacacacacagttccatgacccagcgggcccccaggggcatcaggtgctggtcctcctccctcctggcctcgacccctaagggcttcgcccctcccaggggcctgtaactaagtcgggtcctgccaggcagggggcctgtgttctgtgccccttgggagacaggaactggcgagttcaggtggggtggggacagcacagactgttccaccgttgtgcatattgttgcttctgaaccacaaactgtataaatggatggttttttgacaaaaaaaaaaaaaaaa

SEQ ID NO: 50 - protein, 395 aa, Homo sapiens
MTLRRLRKLQQKEEAAATPDPAARTPDSEVAPAAPVPTPGPPAAAATPGPPADELYAALEDYHPAELYRALAVSGGTLPRRKGSGFRWKNLSQSPEQQRKVLTLEKEDNQTFGFEIQTYGLHHREEQRVEMVTFVCRVHESSPAQLAGLTPGDTIASVNGLNVEGIRHREIVDIIKASGNVLRLETLYGTSIRKAELEARLQYLKQTLYEKWGEYRSLMVQEQRLVHGLVVKDPSIYDTLESVRSCLYGAGLLPGSLPFGPLLAVPGRPRGGARRARGDADDAVYHTCFFGDSEPPALPPPPPPARAFGPGPAETPAVGPGPGPRAALSRSASVRCAGPGGGGGGGAPGALWTEAREQALCGPGLRKTKYRSFRRRLLKFIPGLNRSLEEEESQL

SEQ ID NO: 51 - DNA, 2474 bp, Mus musculus
  CDS (109)..(1914)
gcgaaagttgggggagtgtgctagaaggactgcggagcggagcgcacgcgggaccaggctgcgactgggtcgctggtcccggccacagggagtgggagccggctggagatgccctgtattcaagctcaatatggaacaccagcaacgagcccaggaccgcgtgaccacctgaccggtgatcccctggcccttgagttcggcaagcctaccatggacctggccagccccgagacagcacctgccgcacctgctacactgcccagcttcagcaccttcatggacgggtacaccggagagtttgacaccttcctctaccagctgccggggacgacccagccgtgctcctcagcttgttcctctgcctcctccacgtcttcttcctcatcctcggccacctcccccgcttcggcgtccttcaagtttgaggacttccaggtgtacggctgctacccgggcaccctgagcggcccattagatgagaccctatcctccagcggctctgagtactatggcagtccctgctcagccccctcgccatctacacccaacttccagccgtcccagctttctccctgggacggctcatttggccacttctccccgagccagacttatgaaggcctctgggcatggacagagcagttgcctaaggcttcttcagggcctccgccacctccaaccttcttctccttcagtcctcccactggccccagccccagcctggcccagagttctctgaaattgttcccaccaccagccacccaccagcttggggagggggagagctattccatgccagcagctttccccggcttggcacccacctctccgaaccgtgacacttccggcattctggacgcacccgtgacctccaccaagtcccggagcggggcttcaggtggcagcgagggccgctgtgcagtctgtggtgacaatgcttcgtgtcagcactatggggtccgcacctgtgagggctgcaagggcttcttcaagcgcacagtacagaaaagcgccaagtacatctgcctggcaaacaaggattgccctgtggacaagaggcggcggaaccgctgccagttctgccgcttccagaagtgcctggctgtgggcatggtgaaggaagttgtacggacagacagcctaaaagggcggcggggccggctaccttcaaaacccaagcagcctccagatgcctcccctaccaatcttctcacttccctcatccgggcacacttggactccgggcctagcactgccaaattggactattccaagttccaggaactggtgctgccccgcttcgggaaggaagatgccggtgacgtgcaacaattttatgacttgctctctggttccctggacgttatccgaaagtgggcagaaaaaatccctggcttcattgagctttgcccaggagaccaagacctgttgctagagtctgccttcctggaactcttcatcctccgcctggcataccgatctaaacccggtgaggggaagctcatcttctgctcaggcctggtactacaccagctgcagtgtgcccgtggctttggtgattggattgacaacatcctggccttctcacggtccctgcacagcttgggtgttgatgttcccgcctttgcctgcctgtccgctctggtcctcatcactgatcgacacgggctccaggaccctcgtcgggtggaagagctgcagaatcgcattgctagctgtctgaaggagcacatggctaccgtggcaggagacccacagccggccagctgcctgtcacgtctgctgggcaaactgcctgagcttcggaccctgtgcactcaaggcctgcagcgcatcttttgcctcaagttggaggacttggtaccccctccacctattgtggacaagatctttatggacacattgtctttctgacccctgccctgaacatgtgtgcgcacacgtgcgtgctcttctgtcacccatgtgcctttaagcctatagcccacggacccccagaccaccctacccccagcctggttttgagctaagactgacgtacctcctcactccagaagatggacagagaactcaagacctgggggagggtgtgtattcacgggggtgaccccactatttgtcttatccctccagctcagtcctggccttcgtgtgtttttgtaagataaaccatttttaacacataccactctgttgtaaataagctgacgctactgtaaatacagaaaggaagaggttgagatgggggttgggaggaaggggtggggctcccaccagctgggcgagcctccaactcgagatctcttccgctctccttccatgtgtacataactgtcactcaagaaggtgattgacagattctgatttatatttgtgtattttcctggatttataggatgtgacttttctgattaatatatttaatatattgaataaaaaatagacatgtagttgaaaaaaaaaaaaaaaaaaaa

SEQ ID NO: 52 - protein, 601 aa, Mus musculus
MPCIQAQYGTPATSPGPRDHLTGDPLALEFGKPTMDLASPETAPAAPATLPSFSTFMDGYTGEFDTFLYQLPGTTQPCSSACSSASSTSSSSSSATSPASASFKFEDFQVYGCYPGTLSGPLDETLSSSGSEYYGSPCSAPSPSTPNFQPSQLSPWDGSFGHFSPSQTYEGLWAWTEQLPKASSGPPPPPTFFSFSPPTGPSPSLAQSSLKLFPPPATHQLGEGESYSMPAAFPGLAPTSPNRDTSGILDAPVTSTKSRSGASGGSEGRCAVCGDNASCQHYGVRTCEGCKGFFKRTVQKSAKYICLANKDCPVDKRRRNRCQFCRFQKCLAVGMVKEVVRTDSLKGRRGRLPSKPKQPPDASPTNLLTSLIRAHLDSGPSTAKLDYSKFQELVLPRFGKEDAGDVQQFYDLLSGSLDVIRKWAEKIPGFIELCPGDQDLLLESAFLELFILRLAYRSKPGEGKLIFCSGLVLHQLQCARGFGDWIDNILAFSRSLHSLGVDVPAFACLSALVLITDRHGLQDPRRVEELQNRIASCLKEHMATVAGDPQPASCLSRLLGKLPELRTLCTQGLQRIFCLKLEDLVPPPPIVDKIFMDTLSF

SEQ ID NO: 53 - DNA, 2649 bp, Homo sapiens
  CDS (265)..(2061)
ggctacgaaacttgggggagtgcacagaagaacttcgggagcgcacgcgggaccagggaccaggctgagactcggggcgccagtccgggcaggggcagcgggagccggccgggtagggtgcagcctgaggcttgttcagcagaacaggtgcaagccacattgttgccaagacctgcctgaagccggattctccccactgcctccttcaaccccgcctcttcctcctcctgtgggactgctcccccctcctgtgaggctagatagatgccctgtatccaagcccaatatgggacaccagcaccgagtccgggaccccgtgaccacctggcaagcgaccccctgacccctgagttcatcaagcccaccatggacctggccagccccgaggcagcccccgctgcccccactgccctgcccagcttcagcaccttcatggacggctacacaggagagtttgacaccttcctctaccagctgccaggaacagtccagccatgctcctcagcctcctcctcggcctcctccacatcctcgtcctcagccacctcccctgcctctgcctccttcaagttcgaggacttccaggtgtacggctgctaccccggccccctgagcggcccagtggatgaggccctgtcctccagtggctctgactactatggcagcccctgctcggccccgtcgccctccacgcccagcttccagccgccccagctctctccctgggatggctccttcggccacttctcgcccagccagacttacgaaggcctgcgggcatggacagagcagctgcccaaagcctctgggcccccacagcctccagccttcttttccttcagtcctcccaccggccccagccccagcctggcccagagccccctgaagttgttcccctcacaggccacccaccagctgggggagggagagagctattccatgcctacggccttcccaggtttggcacccacttctccacaccttgagggctcggggatactggatacacccgtgacctcaaccaaggcccggagcggggccccaggtggaagtgaaggccgctgtgctgtgtgtggggacaacgcttcatgccagcattatggtgtccgcacatgtgagggctgcaagggcttcttcaagcgcacagtgcagaaaaacgccaagtacatctgcctggctaacaaggactgccctgtggacaagaggcggcgaaaccgctgccagttctgccgcttccagaagtgcctggcggtgggcatggtgaaggaagttgtccgaacagacagcctgaaggggcggcggggccggctaccttcaaaacccaagcagcccccagatgcctcccctgccaatctcctcacttccctggtccgtgcacacctggactcagggcccagcactgccaaactggactactccaagttccaggagctggtgctgccccactttgggaaggaagatgctggggatgtacagcagttctacgacctgctctccggttctctggaggtcatccgcaagtgggcggagaagatccctggctttgctgagctgtcaccggctgaccaggacctgttgctggagtcggccttcctggagctcttcatcctccgcctggcgtacaggtctaagccaggcgagggcaagctcatcttctgctcaggcctggtgctacaccggctgcagtgtgcccgtggcttcggggactggattgacagtatcctggccttctcaaggtccctgcacagcttgcttgtcgatgtccctgccttcgcctgcctctctgcccttgtcctcatcaccgaccggcatgggctgcaggagccgcggcgggtggaggagctgcagaaccgcatcgccagctgcctgaaggagcacgtggcagctgtggcgggcgagccccagccagccagctgcctgtcacgtctgttgggcaaactgcccgagctgcggaccctgtgcacccagggcctgcagcgcatcttctacctcaagctggaggacttggtgccccctccacccatcattgacaagatcttcatggacacgctgcccttctgacccctgcctgggaacacgtgtgcacatgcgcactctcatatgccaccccatgtgcctttagtccacggacccccagagcacccccaagcctgggcttgagctgcagaatgactccaccttctcacctgctccaggaggtttgcagggagctcaagcccttggggagggggatgccttcatgggggtgaccccacgatttgtcttatcccccccagcctggccccggcctttatgttttttgtaagataaaccgtttttaacacatagcgccgtgctgtaaataagcccagtgctgctgtaaatacaggaagaaagagcttgaggtgggagcggggctgggaggaagggatgggccccgccttcctgggcagcctttccagcctcctgctggctctctcttcctaccctccttccacatgtacataaactgtcactctaggaagaagacaaatgacagattctgacatttatatttgtgtattttcctggatttatagtatgtgacttttctgattaatatatttaatatattgaataaaaaatagacatgtagttggaaaaaaaaaaaaatataaaaaaaaaaaaaaa

SEQ ID NO: 54 - protein, 598 aa, Homo sapiens
MPCIQAQYGTPAPSPGPRDHLASDPLTPEFIKPTMDLASPEAAPAAPTALPSFSTFMDGYTGEFDTFLYQLPGTVQPCSSASSSASSTSSSSATSPASASFKFEDFQVYGCYPGPLSGPVDEALSSSGSDYYGSPCSAPSPSTPSFQPPQLSPWDGSFGHFSPSQTYEGLRAWTEQLPKASGPPQPPAFFSFSPPTGPSPSLAQSPLKLFPSQATHQLGEGESYSMPTAFPGLAPTSPHLEGSGILDTPVTSTKARSGAPGGSEGRCAVCGDNASCQHYGVRTCEGCKGFFKRTVQKNAKYICLANKDCPVDKRRRNRCQFCRFQKCLAVGMVKEVVRTDSLKGRRGRLPSKPKQPPDASPANLLTSLVRAHLDSGPSTAKLDYSKFQELVLPHFGKEDAGDVQQFYDLLSGSLEVIRKWAEKIPGFAELSPADQDLLLESAFLELFILRLAYRSKPGEGKLIFCSGLVLHRLQCARGFGDWIDSILAFSRSLHSLLVDVPAFACLSALVLITDRHGLQEPRRVEELQNRIASCLKEHVAAVAGEPQPASCLSRLLGKLPELRTLCTQGLQRIFYLKLEDLVPPPPIIDKIFMDTLPF

SEQ ID NO: 55 - DNA, 2526 bp, Mus musculus
  CDS (85)..(2232)
ggtttggaccgacttgacaggttcagttggagccagccgatcataggtggccgctgtgacaaagggaaactgtgcttttccagcatgcttactgaccctgatttacctcaggagtttgaaaggatgtcttccaagcgaccagcctctccgtatggggagacagatggagaggtagccatggtgacaagcagacagaaagtggaagaggaggagagtgagaggctcccagcctttcaccttcccttacatgtgagttttcccaacaagcctcactctgaggaatttcagccagtttctctgctgacgcaagagacttgtggtcctaggacccccactgtgcagcacaacacaatggaagtcgatggcaataaagttatgtcttcattagctccatacaactcatctacctcacctcagaaggcggaagaaggtgggcgacagagtggcgagtccgtgtcgagcgcagccctggggactcccgagcggcgcaagggcagcttagcggatgtggtggacaccctgaagcagaggaagatggaagagctgatcaagaatgagccagaagacactcccagtattgaaaagctactctccaaggactggaaagacaagctcctggcaatgggatcagggaactttggagaaataaaagggactcctgagagcctcgctgagaaggagcggcagctcatgggtatgatcaatcagctgaccagtctacgggagcaactcctggctgcccacgatgagcagaagaagttggctgcatctcagatagagaaacagcgtcagcagatggagctggccaagcagcaacaggagcagatcgcgaggcaacagcagcagcttctgcagcaacaacacaaaatcaacttgcttcagcaacagatccagcaggttcaaggtcagctgccgccattgatgattcccgtgttccctcctgaccagcggaccctcgcagcagctgcccagcaaggattcctccttcctcctggtttcagctacaaggctgggtgcagtgacccttaccctgttcagctgatcccaaccactatggcagctgctgctgcagcaacaccaggcttaggcccactccagctgcaggatgaagtggcccagccactgaacctatcagctaagcccaagacctctgatggcaaatcacctgcatcacccacctctcctcacatgccagctctgagaataaacagtggggctggccccctcaaagcctctgtcccggcagcattagctagtccttctgcaagagttagcacaataggctatttaaatgaccacgatgctgtcaccaaggcaatccaagaagctcgccagatgaaagagcaactcaggcgagagcagcaagcgttggacgggaaggtggccgtggtgaacagcataggtctcagcaactgccggacagaaaaggaaaaaacaacactggagagcctgactcagcagttggcagttaaacagaatgaagaagggaaatttagccatggaatgatggatttcaatatgagtggagattctgacggaagcgctggagtctcagagtcaagaatttacagggaatccaggggacgtggtagcaacgagccccacataaagcgtccaatgaatgccttcatggtgtgggcgaaagatgaacggaggaaaatccttcaggcctttcccgacatgcacaattccaacatcagcaagatactgggatctcgctggaaagctatgaccaacctagagaaacagccatactatgaggagcaggcccgcctcagcaaacagcacctggagaagtacccggactacaagtacaagcctaggccaaagcgcacctgtctggtggacggcaagaaactgcgtatcggggagtacaaggccatcatgcggaaccggaggcaggaaatgcgacagtacttcaacgttgggcaacaagcacagatccccatcgcgacggccggagttgtataccccagcgccatcgccatggcaggaatgccgtcccctcacctgccctccgagcactcgagcgtgtccagcagcccggagcccgggatgcccgtgatccagagcacttacggcgccaagggagaggagccccacatcaaggaagagatccaggctgaggacatcaacggagagatttacgaggagtacgatgaggaggaggaggacccggatgtggattatgggagtgacagcgaaaaccacattgcgggacaagccaactgataagggccaacagactgtggtgagccgaggacttgaagaagccctgtccggttcatccttcccagtggccaagcacattaactctctcatacactgactgttattttaactgttagtcttatatagttgggacatcagctgacaaatagacctcagcctcaaaaggctcggaaagaaaagaaagaaagaaagaaagaaaaaaaattacaagcaaaaacgtcatcaagaagaagagatcgaaataagctatgggttaaacagtgccagtaactcagctgctacaccccagcactg

SEQ ID NO: 56 - protein, 715 aa, Mus musculus
MLTDPDLPQEFERMSSKRPASPYGETDGEVAMVTSRQKVEEEESERLPAFHLPLHVSFPNKPHSEEFQPVSLLTQETCGPRTPTVQHNTMEVDGNKVMSSLAPYNSSTSPQKAEEGGRQSGESVSSAALGTPERRKGSLADVVDTLKQRKMEELIKNEPEDTPSIEKLLSKDWKDKLLAMGSGNFGEIKGTPESLAEKERQLMGMINQLTSLREQLLAAHDEQKKLAASQIEKQRQQMELAKQQQEQIARQQQQLLQQQHKINLLQQQIQQVQGQLPPLMIPVFPPDQRTLAAAAQQGFLLPPGFSYKAGCSDPYPVQLIPTTMAAAAAATPGLGPLQLQDEVAQPLNLSAKPKTSDGKSPASPTSPHMPALRINSGAGPLKASVPAALASPSARVSTIGYLNDHDAVTKAIQEARQMKEQLRREQQALDGKVAVVNSIGLSNCRTEKEKTTLESLTQQLAVKQNEEGKFSHGMMDFNMSGDSDGSAGVSESRIYRESRGRGSNEPHIKRPMNAFMVWAKDERRKILQAFPDMHNSNISKILGSRWKAMTNLEKQPYYEEQARLSKQHLEKYPDYKYKPRPKRTCLVDGKKLRIGEYKAIMRNRRQEMRQYFNVGQQAQIPIATAGVVYPSAIAMAGMPSPHLPSEHSSVSSSPEPGMPVIQSTYGAKGEEPHIKEEIQAEDINGEIYEEYDEEEEDPDVDYGSDSENHIAGQAN

SEQ ID NO: 57 - DNA, 2814 bp, Homo sapiens
  CDS (451)..(2379)
gagagtgaaaaaggcgagccaccaaaacccatctccagtctcctcccgggggcccccagcccgcctctgtgccactttgcatcccacgccggaggaggcattaacgagaccgggtaaggctttttaaacggtccaaggtgtagagccatacttcaggaggatcctcagaagttttggacaagcctccccaaatgtggcaggtgctgtgctggccattggtgacccaaagatgatgaaaaatatgttcctgcccacaaggagttagcgacctactgggctttcctcttgctgatgacatgattcctgtttgaatctgttgacaagattctgaaagctgaacagagaattctggcactgcactgggtaggaaaaagcatttcaagaaatagataatatcaaggacatcaggacaccgggagtgggagagattggactgggagactcagcaggatgtcttccaagcgaccagcctctccgtatggggaagcagatggagaggtagccatggtgacaagcagacagaaagtggaagaagaggagagtgacgggctcccagcctttcaccttcccttgcatgtgagttttcccaacaagcctcactctgaggaatttcagccagtttctctgctgacgcaagagacttgtggccataggactcccacttctcagcacaatacaatggaagttgatggcaataaagttatgtcttcatttgccccacacaactcatctacctcacctcagaaggcagaagaaggtgggcgacagagtggcgagtccttgtctagtacagccctgggaactcctgaacggcgcaagggcagtttagctgatgttgttgacaccttgaagcagaggaaaatggaagagctcatcaaaaacgagccggaagaaacccccagtattgaaaaactactctcaaaggactggaaagacaagcttcttgcaatgggatcggggaactttggcgaaataaaagggactcccgagagcttagctgagaaagaaaggcaactcatgggtatgatcaaccagctgaccagcctccgagagcagctgttggctgcccacgatgagcagaagaaactagctgcctctcagattgagaaacagcgtcagcaaatggagctggccaagcagcaacaagaacaaattgcaagacagcagcagcagcttctacagcaacaacacaaaatcaatttgctccagcaacagatccaggttcaaggtcagctgccgccattaatgattcccgtattccctcctgatcaacggacactggctgcagctgcccagcaaggattcctcctccctccaggcttcagctataaggctggatgtagtgacccttaccctgttcagctgatcccaactaccatggcagctgctgccgcagcaacaccaggcttaggcccactccaactgcagcagttatatgctgcccagctagctgcaatgcaggtatctccaggagggaagctgccaggcataccccaaggcaaccttggtgctgctgtatctcctaccagcattcacacagacaagagcacaaacagcccaccacccaaaagcaaggaaaaaacaacactggagagtctgactcagcaactggcagttaaacagaatgaagaaggaaaatttagccatgcaatgatggatttcaatctgagtggagattctgatggaagtgctggagtctcagagtcaagaatttatagggaatcccgagggcgtggtagcaatgaaccccacataaagcgtccaatgaatgccttcatggtgtgggctaaagatgaacggagaaagatccttcaagcctttcctgacatgcacaactccaacatcagcaagatattgggatctcgctggaaagctatgacaaacctagagaaacagccatattatgaggagcaagcccgtctcagcaagcagcacctggagaagtaccctgactataagtacaagcccaggccaaagcgcacctgcctggtggatggcaaaaagctgcgcattggtgaatacaaggcaatcatgcgcaacaggcggcaggaaatgcggcagtacttcaatgttgggcaacaagcacagatccccattgccactgctggtgttgtgtaccctggagccatcgccatggctgggatgccctcccctcacctgccctcggagcactcaagcgtgtctagcagcccagagcctgggatgcctgttatccagagcacttacggtgtgaaaggagaggagccacatatcaaagaagagatacaggccgaggacatcaatggagaaatttatgatgagtacgacgaggaagaggatgatccagatgtagattatgggagtgacagtgaaaaccatattgcaggacaagccaactgataagggtcaaaagattgttgtgaccttaggacttaaagaagccctaactggttcatccttaccagtggccaagcacattaactttctcatacactgactgttactttaactgttagtcttaaatagttgggacatcagctgactaatagacctcagcctcaaaaggcttggaaagaaaaaacaaatacaacaagcaaacaacaatatcaacaacaagagattgaaataagctatgggtaaaataatgccagtaattcagctgctacatccaagcactgaagtcttacccgtcaactttttttttttttaaataaactttatggctgtttgttctacaatgttctagaaattctcactcaggtacacagtgccaacaagtggcttgtgaatgtgttttgttgttttgtgctacaatttttaaaaaaaaaaaaaaaa

SEQ ID NO: 58 - protein, 642 aa, Homo sapiens
MSSKRPASPYGEADGEVAMVTSRQKVEEEESDGLPAFHLPLHVSFPNKPHSEEFQPVSLLTQETCGHRTPTSQHNTMEVDGNKVMSSFAPHNSSTSPQKAEEGGRQSGESLSSTALGTPERRKGSLADVVDTLKQRKMEELIKNEPEETPSIEKLLSKDWKDKLLAMGSGNFGEIKGTPESLAEKERQLMGMINQLTSLREQLLAAHDEQKKLAASQIEKQRQQMELAKQQQEQIARQQQQLLQQQHKINLLQQQIQVQGQLPPLMIPVFPPDQRTLAAAAQQGFLLPPGFSYKAGCSDPYPVQLIPTTMAAAAAATPGLGPLQLQQLYAAQLAAMQVSPGGKLPGIPQGNLGAAVSPTSIHTDKSTNSPPPKSKEKTTLESLTQQLAVKQNEEGKFSHAMMDFNLSGDSDGSAGVSESRIYRESRGRGSNEPHIKRPMNAFMVWAKDERRKILQAFPDMHNSNISKILGSRWKAMTNLEKQPYYEEQARLSKQHLEKYPDYKYKPRPKRTCLVDGKKLRIGEYKAIMRNRRQEMRQYFNVGQQAQIPIATAGVVYPGAIAMAGMPSPHLPSEHSSVSSSPEPGMPVIQSTYGVKGEEPHIKEEIQAEDINGEIYDEYDEEEDDPDVDYGSDSENHIAGQAN

SEQ ID NO: 59 - DNA, 1959 bp, Mus musculus
  CDS (31)..(1344)
agtgtgtcctgggacaccccagtcagctgcatggcctccctgggccccagcccctgggcccctttgtcgaccccagcccctactgcacagctgctgctgttcctgctgctccaagtgtctgctcagccccagggcctgtccgggatgcagggggagccctccttgggagacagttcatctggggaggatgagctgggcgtggatgttctgcccagtgaagaggatgcacctgaagaggcggatccacccgatggggaggatccacctgaagttaattctgaagacaggatggaggagtcccttgggttagaggatctatcgactcccgaggctcctgagcacagtcaaggttcccacggggatgaaaaagggggtggccacagtcattggagctatggaggcaccctactctggccccaggtgtccccagcctgtgctggccgctttcagtccccggtagacatccgcctggagcgcaccgcattctgccgcactctgcaacccctggaacttctgggttatgagctccagcctctcccggaactgagcctatccaacaatggccacacggtacagctgactctgccgccggggctgaagatggctctgggacctgggcaggaataccgggccttgcagttgcatctacactggggaacttcagatcacccaggctcagaacacacagtcaatggtcaccgtttccctgctgagatccacgtggtgcacctcagtactgctttctccgaacttcatgaagccttgggccgcccaggaggcctggcagttttggctgcctttctgcaggagagcccagaagaaaacagtgcttacgaacagttgctgtcccatttggaagaaatctcggaggaaggctccaagattgagatcccaggtttggatgtatctgcactgttgccctcggacctcagccgctactaccgatatgaagggtctctgaccacacctccctgcagccagggggtcatctggactgtgttcaatgagacagtgaaactgagcgctaagcagctccatactctctccgtttccttgtggggacctcgtgattctcggctacaactgaacttccgagccacgcaacccttgaatgggcgaacgattgaggcttccttccctgctgcagaggatagcagccctgagccagtccatgtgaattcctgcttcactgctggtgacatcctagccttggtgtttggccttctctttgctgtcaccagcatcgccttcctcctacagctgaggaggcagcacaggcacagaagtgggaccaaagatagagttagctacagtccggcagagatgacagaaactggagcctaataaggagaaaccagctaaaggaatctggaaaccttgtcctgtcctgcctgttatacagcctcctttttaaccactacgaaatcatttccatctttttaactttcttctttccttaaagtactgtgataggagctaaccgtgaaaaaacaagattgggctcaattcttgctgggttttgatggttttgaagacagggtttctctgtgtagccctggctgtcctgggactcactctgtagcctaggctggccttgaactcagagatccgattgcctctgccttcttagtgccaggactgagggtatgtaccactaccaccgccaccccgcaattcttgcttttattctgcagtcaaatgctctaccactgagctacacacccctccatcttactatgcaggcagactggccttgcattgtgtatgtatggtgatggagtttgaagccagggtcttacctactaggcaagcgttctatcactgacctaagcctctctagtcccttctcttgaaccctctattctcctgtcccagcctcttgaatgctgggattacaggaatgcaccatcatgcttggtcatcccaattatttaaaaaaaatatataaatcttcctagt

SEQ ID NO: 60 - protein, 437 aa, Mus musculus
MASLGPSPWAPLSTPAPTAQLLLFLLLQVSAQPQGLSGMQGEPSLGDSSSGEDELGVDVLPSEEDAPEEADPPDGEDPPEVNSEDRMEESLGLEDLSTPEAPEHSQGSHGDEKGGGHSHWSYGGTLLWPQVSPACAGRFQSPVDIRLERTAFCRTLQPLELLGYELQPLPELSLSNNGHTVQLTLPPGLKMALGPGQEYRALQLHLHWGTSDHPGSEHTVNGHRFPAEIHVVHLSTAFSELHEALGRPGGLAVLAAFLQESPEENSAYEQLLSHLEEISEEGSKIEIPGLDVSALLPSDLSRYYRYEGSLTTPPCSQGVIWTVFNETVKLSAKQLHTLSVSLWGPRDSRLQLNFRATQPLNGRTIEASFPAAEDSSPEPVHVNSCFTAGDILALVFGLLFAVTSIAFLLQLRRQHRHRSGTKDRVSYSPAEMTETGA

SEQ ID NO: 61 - DNA, 1639 bp, Homo sapiens
  CDS (105)..(1484)
  misc_difference (201)..(201): ′A′ in cDNA is ′G′ in the human genome; amino acid difference ′M′ in cDNA, ′V′ in the human genome.  The chimpanzee genome agrees with the human genomic sequence and not the cDNA.
cagcctgctcccctccaggcttgctcctcccccacccagctctcgtttccaatgcacgtacagcccgtacacaccgtgtgctgggacaccccacagtcagccgcatggctcccctgtgccccagcccctggctccctctgttgatcccggcccctgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgcaaagaaattttttaaaataaatatttataataaaaaaaaaaaaaaaaaaaaaaaaa

SEQ ID NO: 62 - protein, 459 aa, Homo sapiens
MAPLCPSPWLPLLIPAPAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 63 - DNA, 2060 bp, Mus musculus
  CDS (45)..(1550)
attttgctgagcctgtcacttgtgttccaggagccaaaccagcaatgtcttcacctgcacaacctgcagtccctgccccgctggccaacttgaagattcaacataccaagatctttataaacaatgaatggcatgattcggtgagcagcaagaaatttcctgtccttaaccctgcaactgaggaggtcatctgccacgtggaagaaggggacaaggctgatgttgacaaagctgtgaaggccgcaagacaggctttccagattggctccccatggcgcaccatggatgcttcagagaggggccgcctgctgaacaagctggctgacttaatggagagagatcgtctgctgctggctacaatggaatcgatgaatgctgggaaagtctttgctcatgcatacctgttggatgtagagatcagcataaaagcattacagtacttcgcaggctgggctgacaagatccatggccaaacaatacccagtgatggaaacatattcacttatacaaggcgtgaacctattggggtgtgtggccaaatcatcccttggaatggtccattgattatattcacttggaagttaggccctgcccttagctgtgggaacactgtggttgtcaagccagcagagcaaactcctctcacagctcttcacatggcatctttaataaaagaggcagggtttcctcctggcgtggtgaacattgtccctggttatgggccaactgcagggggagccatctcctcccacatggacatcgacaaagtgtccttcacaggatcaacagaggttggcaaattaatcaaggaagctgcagggaaaagcaatctgaagagagtcaccctggagctggggggaaagagcccttgcattgtgtttgcagatgccgacttggacagtgctgttgagtttgcacaccaaggagtgttcttccaccagggtcagatttgtgttgcagcgtccaggctttttgttgaggaatcaatttacgatgagtttgtgaggaggagtgtggagcgggctaagaaatacattctaggaaatcctctgaactccggaataaatcaaggtcctcagattgacaaggaacaacacaataaaatacttggtctcattgagagtgggaagaaagaaggagccaagctggagtgtggtggaggtcgctgggggaacaaaggcttctttgtccaacccacagtcttctccaatgtgactgatgagatgcgcattgccaaagaggagatatttggaccagtgcaacaaatcatgaagtttaaatctatggatgatgtgatcaagagagcaaacaacactacctatggtttagcagcaggagtcttcactaaagacctggataaggccatcactgtgtcatctgctctgcaggctgggatggtgtgggtgaactgctatttggctgtccctgtccaatgcccatttggtggattcaagatgtctggaaatgggcgagaactgggcgaacatggtctttatgaatacactgagctcaagacagttgcaatgcaaatatctcagaagaactcataaagaagccagcagagtgaagagaaactctcagcagtggctgtacatctcatacagtcaccagaaagtggtgttttgctataagttcttcttctgttgagttctttaaacgtaaggaattcatcagtattactgttaatcatagaaaagacgtagcttaatctggcagaatcagagtcttctaatccgtgaacccaattcatatcccagaacaaaaggatacattaaggtgcaagctctctgtaactctgtcatgataaggtgctttccattgtagctacttgtctagagtacacacttggcaaggaggaccagtcctgatttaagctctgtcccacactgattccttgaactacgtaccatgcatgataactgaaaagttagctgctctattcctcggggttattcctctgagtgttgtgaaatattttccagaatgccatgactgcttgtcaaatgaaatgcctccctgtaattcgaatgcaaagcttaataaaggcaatctcgcatgat

SEQ ID NO: 64 - protein, 501 aa, Mus musculus
MSSPAQPAVPAPLANLKIQHTKIFINNEWHDSVSSKKFPVLNPATEEVICHVEEGDKADVDKAVKAARQAFQIGSPWRTMDASERGRLLNKLADLMERDRLLLATMESMNAGKVFAHAYLLDVEISIKALQYFAGWADKIHGQTIPSDGNIFTYTRREPIGVCGQIIPWNGPLIIFTWKLGPALSCGNTVVVKPAEQTPLTALHMASLIKEAGFPPGVVNIVPGYGPTAGGAISSHMDIDKVSFTGSTEVGKLIKEAAGKSNLKRVTLELGGKSPCIVFADADLDSAVEFAHQGVFFHQGQICVAASRLFVEESIYDEFVRRSVERAKKYILGNPLNSGINQGPQIDKEQHNKILGLIESGKKEGAKLECGGGRWGNKGFFVQPTVFSNVTDEMRIAKEEIFGPVQQIMKFKSMDDVIKRANNTTYGLAAGVFTKDLDKAITVSSALQAGMVWVNCYLAVPVQCPFGGFKMSGNGRELGEHGLYEYTELKTVAMQISQKNS

SEQ ID NO: 65 - DNA, 2116 bp, Homo sapiens
  CDS (54)..(1559)
atcagaaccaaattgctgagccagtcacctgtgttccaggagccgaatcagaaatgtcatcctcaggcacgccagacttacctgtcctactcaccgatttgaagattcaatatactaagatcttcataaacaatgaatggcatgattcagtgagtggcaagaaatttcctgtctttaatcctgcaactgaggaggagctctgccaggtagaagaaggagataaggaggatgttgacaaggcagtgaaggccgcaagacaggcttttcagattggatccccgtggcgtactatggatgcttccgagagggggcgactattatacaagttggctgatttaatcgaaagagatcgtctgctgctggcgacaatggagtcaatgaatggtggaaaactctattccaatgcatatctgaatgatttagcaggctgcatcaaaacattgcgctactgtgcaggttgggctgacaagatccagggccgtacaataccaattgatggaaatttttttacatatacaagacatgaacctattggtgtatgtggccaaatcattccttggaatttcccgttggttatgctcatttggaagatagggcctgcactgagctgtggaaacacagtggttgtcaaaccagcagagcaaactcctctcactgctctccacgtggcatctttaataaaagaggcagggtttcctcctggagtagtgaatattgttcctggttatgggcctacagcaggggcagccatttcttctcacatggatatagacaaagtagccttcacaggatcaacagaggttggcaagttgatcaaagaagctgccgggaaaagcaatctgaagagggtgaccctggagcttggaggaaagagcccttgcattgtgttagctgatgccgacttggacaatgctgttgaatttgcacaccatggggtattctaccaccagggccagtgttgtatagccgcatccaggatttttgtggaagaatcaatttatgatgagtttgttcgaaggagtgttgagcgggctaagaagtatatccttggaaatcctctgaccccaggagtcactcaaggccctcagattgacaaggaacaatatgataaaatacttgacctcattgagagtgggaagaaagaaggggccaaactggaatgtggaggaggcccgtgggggaataaaggctactttgtccagcccacagtgttctctaatgttacagatgagatgcgcattgccaaagaggagatttttggaccagtgcagcaaatcatgaagtttaaatctttagatgacgtgatcaaaagagcaaacaatactttctatggcttatcagcaggagtgtttaccaaagacattgataaagccataacaatctcctctgctctgcaggcaggaacagtgtgggtgaattgctatggcgtggtaagtgcccagtgcccctttggtggattcaagatgtctggaaatggaagagaactgggagagtacggtttccatgaatatacagaggtcaaaacagtcacagtgaaaatctctcagaagaactcataaagaaaatacaagagtggagagaagctcttcaatagctaagcatctccttacagtcactaatatagtagattttaaagacaaaatttttcttttcttgatttttttaaacataagctaaatcatattagtattaatactacccatagaaaacttgacatgtagcttcttctgaaagaattatttgccttctgaaatgtgacccccaagtcctatcctaaataaaaaaagacaaattcggatgtatgatctctctagctttgtcatagttatgtgattttcctttgtagctacttttgcaggataataattttatagaaaaggaacagttgcatttagcttctttcccttagtgactcttgaagtacttaacatacacgttaactgcagagtaaattgctctgttcccagtagttataaagtccttggactgttttgaaaagtttcctaggatgtcatgtctgcttgtcaaaagaaataatccctgtaatatttagctgtaaactgaatataaagcttaataaaaacaaccttgcatgaaaaaaaaaaaaaaaaaaaaa

SEQ ID NO: 66 - protein, 501 aa, Homo sapiens
MSSSGTPDLPVLLTDLKIQYTKIFINNEWHDSVSGKKFPVFNPATEEELCQVEEGDKEDVDKAVKAARQAFQIGSPWRTMDASERGRLLYKLADLIERDRLLLATMESMNGGKLYSNAYLNDLAGCIKTLRYCAGWADKIQGRTIPIDGNFFTYTRHEPIGVCGQIIPWNFPLVMLIWKIGPALSCGNTVVVKPAEQTPLTALHVASLIKEAGFPPGVVNIVPGYGPTAGAAISSHMDIDKVAFTGSTEVGKLIKEAAGKSNLKRVTLELGGKSPCIVLADADLDNAVEFAHHGVFYHQGQCCIAASRIFVEESIYDEFVRRSVERAKKYILGNPLTPGVTQGPQIDKEQYDKILDLIESGKKEGAKLECGGGPWGNKGYFVQPTVFSNVTDEMRIAKEEIFGPVQQIMKFKSLDDVIKRANNTFYGLSAGVFTKDIDKAITISSALQAGTVWVNCYGVVSAQCPFGGFKMSGNGRELGEYGFHEYTEVKTVTVKISQKNS

SEQ ID NO: 67 - DNA, 4751 bp, Mus musculus
  CDS (47)..(1486)
gcaaagcaccatttctccaagttggggggctcagaggggagccatcatgagcgatgttaccattgtgaaagaaggttgggttcagaagaggggagaatatataaaaaactggaggccaagatacttccttttgaagacagatggctcattcataggctataaggagaaacctcaagatgtggacttaccttatcccctcaacaacttctcagtggcaaaatgtcagttaatgaaaacagaacgaccaaagccaaatacatttattatcagatgtcttcagtggaccactgttatagagagaacatttcatgtagatacaccagaggaaagagaagagtggacggaagctatccaagccgtagccgaccgattgcagaggcaagaggaggagaggatgaattgtagcccagcctcacagattgataatataggagaagaagagatggatgcgtctacaacccatcataaaagaaagacgatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactcttactgaaagcagagtactaaagaacaccagacatccatttttaacatccttgaaatattccttccagacaaaagaccgtttgtgttttgtgatggaatatgttaatggcggagagctgtttttccatttgtcgagagagcgagtgttctctgaggaccgcacacgtttctatggtgcagaaattgtctctgctttggactatctacattctggaaagattgtgtaccgtgatctcaagttggagaatttgatgctagataaggatggccatataaaaattacggattttgggctttgcaaagaagggatcacagatgcagctaccatgaagacattctgtggcacaccagagtacctggcaccagaggtattagaagataatgactatggccgagccgtggactggtggggcttaggtgttgtcatgtatgaaatgatgtgtggaaggttgcctttctacaaccaggatcatgagaaactctttgaattaatactaatggaagacattaaattcccccgaacactctcttcagatgcaaaatcattgctttcagggctcttgataaaggatccaaataaacgccttggtggagggccagatgatgcaaaagaaatcatgaggcatagttttttttctggagtaaactggcaagatgtatatgacaaaaagcttgtacctccttttaagcctcaagtaacatctgaaacagacacccgatattttgatgaagaatttacagctcagactattacaataacaccacctgaaaagtatgacgacgacggcatggacggcatggacagcgagcggcggccacacttccctcagttctcctactctgcaagcggacgggaataagttcctttcagtctgtttctacactgtcatcttagactttgcctgagactgattcctggacatctctaccagtcctcgctcttacagttagcaggggcaccttctgacatccctgaccagccaagggtcttcaccctcaccacctttcactcacatgaaaccatatacacagacactccagttttgtttttgcatgaaattgtatctcagtctaaggtctcatgctgttgctgctactgtcttactattatagcaaccttcagaagtaatttcacaatctttgggagtcatgagcccattgttcatttgtgcatcaagtgtcatcttttggtttttggttttccctagcagtgaaggctaaatgagatacactgattctaggtacattgttaactttctaggagataaatcaagaactaattagactaagaagatttagtttatatttctgaacaagcaattgttgaagggtggtggtgtgtgtatgtaagagtttagcagtgtatgtctgaccagcacatgaagtgtgtatcactgacatttggctggaaaatcttaggaaacattgagaggactgaagggccaatgagatacacatttttttttttttaaatcctgctggctattgtggaggaagcccagtgccacccatggatgcccaggtttcaagggcaaatggacattgttcaaccaagtactgacggtgcagagtcccctagagagtcttaggatctactttgtaccagagagtattttcccctgggcttggcaacctttttcccttctttatatttatcacctctgatggctgaagaatgtagacagtataatgatcctgctgtcgccaaaatctatacaccaaggtgaacaggtgtttgccttaccagttttggagtttttactttcagttataggtaaattagctttttctcagatgttaaaaccttgaatgtcctttatggttttgtttatattacagtagtatttattttttagtggtaagaattgtgttgtcatgttagcaagcgcagctccaattcacagatcattgcctgcgttttcttttgacccatgtgcaaggaatgtacacacccattagaatcatgcactttttctcctatgttaaaaacaatggtaaggctctgggactgtatctgttggttagaatctggctagaatctctgggagaagagatgctgtgatttaaccccttggagctgaaatgagagcatccccaactacccagtgccaagggatggatgtgagaggatagtctggcagaaagtaatggagaaagctgagacccaaatgatgatgagaaatgaactctactcggcaaaatgaacacatcacaagtattcacacagtaggaagtaaaggataagaagctgatgtgtttattcttatcaaggtacattcattactgaatcaagccatcagggaaaacaacaaaacaaaacacctctagcttttatttttctgtacatattcatgtctcccaacccaaacatttctcactgagaggggacatgtatgcaaacctcatctttctccttcattaatgatgatcttcagattaaccctttggtgctaggagctgacactttccaaagcagactatgacgtccaaggatcaaagcctctcttgcagcaagcaagagcaactgctgctccaaggcgtgatcatggaagtggaagaccacagtgtgacacagtggtactgcattgtctttttcagtaggaagtcatgttctcatgcaagtgtcttggcctctaggctcagacttagagaagcagagccgctgctggcagtggctggccgctgggactgctggggtgtaaagggcattttcctaaggcagctaagacagactcagacatagatttttgtcctaatttttcatgtttctacctgggtgagtttaccctcagtgggaagtaggaagttaaacaggtgtattcttacatctgctgtcttctgttttcctacatagaagtatgttgaatgttgcacattgacaaggaagtagattagatggtaagtccttttaagttaccctgcatccattctgtaagaaaggaggaggagaggaagaaaagcaagctgagcctggccgatgttcagaacatttactccctcagagggagcagacactgtacactaagcacacagcttcttttgttttgtctctccttccaagtcttaaggtaatttcatgaccttggccaaggatactttgtgaagattaatgaggggacattgacaatgcctcaggctggccactcctcacactggcccttggtccccagccattcaaggggttactcatcccatccctgactacctgagtgtcactacaggtgggttcttggctttgagttcaagaattctttccttacaccctccaccagatgtcagtggaaaggcagaagtagaaccgaggtgtgggaggagattctaaaagcttgtgttgcccaggcctctagcttagggttctggcaattgagacggcctgaaacctgacagcatcatgtccgggcagctctggctttgtgagtccagctcctaccaactccaccttgacctcttgtctccatgcagggctgtctgggaggaaactggccacttctgctcagactgctgccagcgctctcacagctttgctctctacactaatgacatagattattccagtattgttcccatttcccacctgacctccagcttctcggagctgacttcttgcaggggccacatgcttctttccctcactaactgcagggtctccaccacacctcagtgtacacactttgctgctaccgtctgtactgtctacatcacggttcccttagcttgctcctggtagtgcattacaggcaagcatgaaatgtaaagtatttatttaaataaaaaggaaacctctgaattggtcatcgagtcacctccctgtagatttgtagtctgtgacatatttcgactttctagtcctgctagatccatataagatctggtcatctggttaagggtttgaagcagacatagctatgaacccggatggtatcatcactgtcacggctggccacactacagatgtttatgtccttacgtttatgacccacagggtgtgaagtaacttccaagaacctgattttgtacactgtccactctcagcatctcacactctctgatagggacacacattacttccttcgtacagacagcttaaataaagccctatgtcaatctgcaaaaaaaaaaaaaaaa

SEQ ID NO: 68 - protein, 479 aa, Mus musculus
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPASQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMLDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKYDDDGMDGMDSERRPHFPQFSYSASGRE

SEQ ID NO: 69 - DNA, 1623 bp, Homo sapiens
  CDS (84)..(1481)
cagcagcagcagagaatccaaaccctaaagctgatatcacaaagtaccatttctccaagttgggggctcagaggggagtcatcatgagcgatgttaccattgtgaaagaaggttgggttcagaagaggggagaatatataaaaaactggaggccaagatacttccttttgaagacagatggctcattcataggatataaagagaaacctcaagatgtggatttaccttatcccctcaacaacttttcagtggcaaaatgccagttaatgaaaacagaacgaccaaagccaaacacatttataatcagatgtctccagtggactactgttatagagagaacatttcatgtagatactccagaggaaagggaagaatggacagaagctatccaggctgtagcagacagactgcagaggcaagaagaggagagaatgaattgtagtccaacttcacaaattgataatataggagaggaagagatggatgcctctacaacccatcataaaagaaagacaatgaatgattttgactatttgaaactactaggtaaaggcacttttgggaaagttattttggttcgagagaaggcaagtggaaaatactatgctatgaagattctgaagaaagaagtcattattgcaaaggatgaagtggcacacactctaactgaaagcagagtattaaagaacactagacatccctttttaacatccttgaaatattccttccagacaaaagaccgtttgtgttttgtgatggaatatgttaatgggggcgagctgtttttccatttgtcgagagagcgggtgttctctgaggaccgcacacgtttctatggtgcagaaattgtctctgccttggactatctacattccggaaagattgtgtaccgtgatctcaagttggagaatctaatgcgggacaaagatggccacataaaaattacagattttggactttgcaaagaagggatcacagatgcagccaccatgaagacattctgtggcactccagaatatctggcaccagaggtgttagaagataatgactatggccgagcagtagactggtggggcctaggggttgtcatgtatgaaatgatgtgtgggaggttacctttctacaaccaggaccatgagaaactttttgaattaatattaatggaagacattaaatttcctcgaacactctcttcagatgcaaaatcattgctttcagggctcttgataaaggatccaaataaacgccttggtggaggaccagatgatgcaaaagaaattatgagacacagtttcttctctggagtaaactggcaagatgtatatgataaaaagcttgtacctccttttaaacctcaagtaacatctgagacagatactagatattttgatgaagaatttacagctcagactattacaataacaccacctgaaaaatgtcagcaatcagattgtggcatgctgggtaactggaaaaaataataaaaatcggcttcctacagccagcagcacagtcacccatggaactgttggctttggattaaatgtggaattgaacgactacccagaagtgttctggaaagaagcgagatgtgtggcctgcctcaccgtcctcacccatcaa

SEQ ID NO: 70 - protein, 465 aa, Homo sapiens
MSDVTIVKEGWVQKRGEYIKNWRPRYFLLKTDGSFIGYKEKPQDVDLPYPLNNFSVAKCQLMKTERPKPNTFIIRCLQWTTVIERTFHVDTPEEREEWTEAIQAVADRLQRQEEERMNCSPTSQIDNIGEEEMDASTTHHKRKTMNDFDYLKLLGKGTFGKVILVREKASGKYYAMKILKKEVIIAKDEVAHTLTESRVLKNTRHPFLTSLKYSFQTKDRLCFVMEYVNGGELFFHLSRERVFSEDRTRFYGAEIVSALDYLHSGKIVYRDLKLENLMRDKDGHIKITDFGLCKEGITDAATMKTFCGTPEYLAPEVLEDNDYGRAVDWWGLGVVMYEMMCGRLPFYNQDHEKLFELILMEDIKFPRTLSSDAKSLLSGLLIKDPNKRLGGGPDDAKEIMRHSFFSGVNWQDVYDKKLVPPFKPQVTSETDTRYFDEEFTAQTITITPPEKCQQSDCGMLGNWKK

SEQ ID NO: 71 - DNA, 1799 bp, Mus musculus
tatggaatagggcatttccagaggggaaactgggaaaggagataaaatttcaagtgtaaataaataaaatattcaatacaaaaaaagtagtctggaccaagagaacaagagactgacaaagccaaagtatctgtgttataacaataacaatattgttaattataacagttatataaaaatcaggctagggaaagggaaagcccagccccaggttgggggcgggatgagaagtgttgggaggaacccccagtaaggagtggtgctgcccaagctgggaggagctggccagcagtggcttccataagcatgtcagccagctattggtttctgaaaggtagcctctacaaacctgagttgctgagtctggtgtggtagcaagctctggttgtcatacacaaaatgctcaaatgaaggccagctgataaaaagaaaaacactaagcctaatggtatatatctatcgtctgccaccttttacccaaggaatacatgtcaagtcaacatgttaggagtttcagcaatatttactaatctttgctatgagtccagtgtcttagttaacgtgtagtgctgctatcctgttgtttactgcattttattcttgctttgtctctcatcactgtaaatcataatcgataaagcgcatcactatgacaagcgtcatgcttttctggaggtcactcttcctgggagtgcgtcaatgtaaaatgtgtcttcctattttaaaactttcgatgagtctttggcatctacataacagctcagtctccagtttttcccccagtcttccacttctgtcaagaaatcacaagtccaggtcttgcctccatttaataattgtgctccactggagagtttcaagtattcaagtctacagctcatatgtcttcataggcgtccctaactgaaattaatggagaagatgacaaatttataaaacttcattttttgtcttctgaaaatatgtattcatcaatctttttaaaactgtctctgtgattttgtttattattcatctcactgctcgcaaaacggacacaaaatgtcagagaaatatttgctgtgttttccaaaggggaatttagagagggagtaaagaataagttgtggggtagataataccgattttaaatgctgttgctagacaacagctgctgattatgggaagtttctgaagttctccatataagttgtctcgtgtatgaagtgaccaataactttaaaaacttttggatcgtaaatctgtcaagagacagattgaactagagtatgtaaagtatttgtagatcttaacatacaatgacctgtgtactgaagttaatgatgttaattagctattgttaattacaataacttctgtaatctcagaagctcacattgcacttgactcagccccgctgtcaatcaatattatgctggtgaatgaataacatgagttaatgattactgtaagtagcacaaattccgtagtgccatttttataatatttacccccgttgatttccttattattatggcttatttcttcagaaattatctttatctgtgcaagagaattcactccaactcagtcagccatatagttagtagagctctgccagatctgtggagcagagagaaaatgaatgggctggaaatgaagttattagtggagtggcctcatgccacggcagcttctcttttgttaatattaaaaaaacgatttcttcacacagcatccaaaatagctacttaacaatataaagtggggcatgtttctccattccataaaagttcccagtggcttcttttcacacacag

SEQ ID NO: 72 - DNA, 715 bp, Mus musculus
  CDS (47)..(556)
tcacccttgctaatcactcctcacagtgacctcaagtcctgcaggcatgtacagcatgcagctcgcatcctgtgtcacactgacacttgtgctccttgtcaacagcgcacccacttcaagccccacttcaagccccacttcaagctctacagcggaagcacagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataactatgtacctcctgcttacaacacataaggctctctatttatttaaatatttaactttaatttatttttggatgtattgtttactatcttttgtaactactagtcttcagatgataaatatggatctttaaagattctttttgtaagccccaagggctc

SEQ ID NO: 73 - protein, 169 aa, Mus musculus
MYSMQLASCVTLTLVLLVNSAPTSSPTSSPTSSSTAEAQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 74 - DNA, 822 bp, Homo sapiens
  CDS (56)..(517)
agttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaaaaaaaaaaaaaaaaaaaaaaaaa

SEQ ID NO: 75 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 76 - DNA, 916 bp, Mus musculus
  CDS (138)..(599)
gagctcagcaagcgctctcccccaattcccttagccaaagtggacgccaccgaacagacagacctaggctaagaggtttgatgtctctggctacccgactttgaaaattttccgcaaaggaaggccttttgactacaatggcccacgagagaaaggctaaggtcctgaggaggatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaatgaggaagcccaggccagctctgaatccagcttctcagactgctgcttttgtgcctgcgtaatgagccaggaactcggaatttctgccttaaagggaccaagagatgtggcacagccacagttggagggcagtatagccctctgaaaacgctgactcagcttggacagcggcaagacaaacgagagatattttctactgatagggaccattatatttatttatatatttatattttttaaatatttatttatttatttatttaattttgcaactctatttattgagaatgtcttaccagataataaattattaaaacttt

SEQ ID NO: 77 - protein, 153 aa, Mus musculus
MAHERKAKVLRRMWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 78 - DNA, 789 bp, Homo sapiens
  CDS (33)..(467)
acacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggcacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatagcttctaaaaaaaaaaa

SEQ ID NO: 79 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 80 - DNA, 1171 bp, Mus musculus
  CDS (58)..(534)
atccacctcacacgaggcacaagtgcacccagcaccagctgatcaggacgcgcaaacatgagtccagggagagcttcatctgtgtctctgatgctgttgctgctgctgagcctggcggctacagtgaaggcagcagcgatcatccctcaaagctcagcgtgtccaaacactgaggccaaggacttcctccagaatgtgaaggtcaacctcaaagtctttaactcccttggcgcaaaagtgagctccagaaggccctcagactacctcaaccgttccacgtcaccctggactctccaccgcaatgaagaccctgatagatatccctctgtgatctgggaagctcagtgccgccaccagcgctgtgtcaatgcggagggaaagctggaccaccacatgaattctgttctcatccagcaagagatcctggtcctgaagagggagcctgagagctgccccttcactttcagggtcgagaagatgctggtgggtgtgggctgcacctgcgtggcctcgattgtccgccaggcagcctaaacagagacccgcggctgacccctaagaaacccccacgtttctcagcaaacttacttgcatttttaaaacagttcgtgctattgattttcagcaaggaatgtggattcagaggcagattcagaattgtctgccctccacaatgaaaagaaggtgtaaaggggtcccaaactgcttcgtgtttgtttttctgtggactttaaattatttgtgtatttacaatatcccaagatagctttgaagcgtaacttattttaatgaagtatctacattattattatgtttctttctgaagaagacaaaattcaagactcagaaattttattatttaaaaggtaaagcctatatttatatgagctatttatgaatctatttatttttcttcagtatttgaagtattaagaacatgattttcagatctacctagggaagtcctaagtaagattaaatattaatggaaatttcagctttactatttgtttatttaaggttctctcctctgaatggggtgaaaaccaaacttagttttatgtttaataactttttaaattattgaagattcaaaaaattggataatttagctccctactctgttttaaaaaaaaattgtaacaatatcactgtaataataaagttttgg

SEQ ID NO: 81 - protein, 158 aa, Mus musculus
MSPGRASSVSLMLLLLLSLAATVKAAAIIPQSSACPNTEAKDFLQNVKVNLKVFNSLGAKVSSRRPSDYLNRSTSPWTLHRNEDPDRYPSVIWEAQCRHQRCVNAEGKLDHHMNSVLIQQEILVLKREPESCPFTFRVEKMLVGVGCTCVASIVRQAA

SEQ ID NO: 82 - DNA, 1859 bp, Homo sapiens
  CDS (46)..(513)
gcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

SEQ ID NO: 83 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 84 - DNA, 707 bp, Mus musculus
  CDS (50)..(592)
ggggttcctggcgggtggcagctgcgggcctgccgcctgacttggtgggatggactggccgcacagcctgctcttcctcctggccatctccatcttcctggcgccaagccacccccggaacaccaaaggcaaaagaaaagggcaagggaggcccagtcccttggcccctgggcctcatcaggtgccgctggacctggtgtctcgagtaaagccctacgctcgaatggaagagtatgagcggaaccttggggagatggtggcccagctgaggaacagctccgagccagccaagaagaaatgtgaagtcaatctacagctgtggttgtccaacaagaggagcctgtccccatggggctacagcatcaaccacgaccccagccgcatccctgcggacttgcccgaggcgcggtgcctatgtttgggttgcgtgaatcccttcaccatgcaggaggaccgtagcatggtgagcgtgccagtgttcagccaggtgccggtgcgccgccgcctctgtcctcaacctcctcgccctgggccctgccgccagcgtgtcgtcatggagaccatcgctgtgggttgcacctgcatcttctgagccaaccaccaacccggtggcctctgcaacaaccctccctccctgcacccactgtgaccctcaaggctgataaacagtaaacgctgttctttgtaaaggaaaaaaaaaaaaaaaa

SEQ ID NO: 85 - protein, 180 aa, Mus musculus
MDWPHSLLFLLAISIFLAPSHPRNTKGKRKGQGRPSPLAPGPHQVPLDLVSRVKPYARMEEYERNLGEMVAQLRNSSEPAKKKCEVNLQLWLSNKRSLSPWGYSINHDPSRIPADLPEARCLCLGCVNPFTMQEDRSMVSVPVFSQVPVRRRLCPQPPRPGPCRQRVVMETIAVGCTCIF

SEQ ID NO: 86 - DNA, 711 bp, Homo sapiens
  CDS (51)..(593)
tggggttccaggcgggcagcagctgcaggctgaccttgcagcttggcggaatggactggcctcacaacctgctgtttcttcttaccatttccatcttcctggggctgggccagcccaggagccccaaaagcaagaggaaggggcaagggcggcctgggcccctggcccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctgaatcacctggcccagaagccaggccagcagcccgagaccatcctccttgcacctttgtgccaagaaaggcctatgaaaagtaaacactgacttttgaaagcaaaaaaaaaaaaaaaaaa

SEQ ID NO: 87 - protein, 180 aa, Homo sapiens
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF